(19) United States
(12) Patent Application Publication
(10) Pub. No.: US 2019/0307776 A1
(43) Pub. Date: Oct. 10, 2019

(54) AZA-SUBSTITUTED INHIBITORS OF HUMAN IMMUNODEFICIENCY VIRUS REPLICATION

(71) Applicant: VIIV HEALTHCARE UK (NO. 5) LIMITED, Brentford, Middlesex (GB)

(72) Inventors: Jie CHEN, Wallingford, CT (US); Yan CHEN, Wallingford, CT (US); Alicia REGUEIRO-REN, Wallingford, CT (US); Sing-Yuen SIT, Wallingford, CT (US); Jacob SWIDORSKI, Wallingford, CT (US)

(73) Assignee: ViiV HealthCare UK (No. 5) Limited, Brentford, Middlesex (GB)

(21) Appl. No.: 16/308,016

(22) PCT Filed: Jun. 28, 2017
(86) PCT No.: PCT/IB2017/053878

§ 371 Date: Dec. 7, 2018

Related U.S. Application Data
(60) Provisional application No. 62/356,845, filed on Jun. 30, 2016.

(51) Int. Cl. A61K 31/58; A61K 31/5365; C07J 71/00; A61P 31/18

(52) U.S. Cl. CPC A61K 31/58; A61P 31/18; C07J 71/0047; A61K 31/5365

(57) ABSTRACT

Compounds having drug and bio-affecting properties, their pharmaceutical compositions and methods of use are set forth. In particular, aza-substituted triterpenoid compounds that possess unique antiviral activity are provided as HIV maturation inhibitors, as represented by compounds of Formula I:

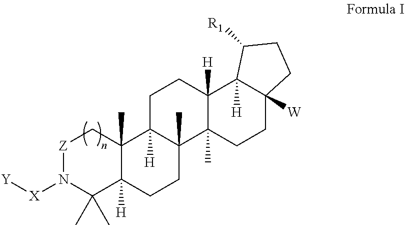

These compounds are useful for the treatment of HIV and AIDS.

### FIELD OF THE INVENTION

The invention relates to compounds, compositions, and methods for the treatment of human immunodeficiency virus (HIV) infection. More particularly, the invention provides novel aza-substituted triterpenoid compounds as inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. HIV infection remains a major medical problem, with an estimated 45-50 million people infected worldwide at the end of 2011, 3.3 million of them under the age of 15. In 2011, there were 2.5 million new infections, and 1.7 million deaths from complications due to HIV/AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

Despite the armamentarium of agents and drug combinations, there remains a medical need for new anti-retroviral agents, due in part to the need for chronic dosing to combat infection. Significant problems related to long-term toxicities are documented, creating a need to address and prevent these co-morbidities (e.g. CNS, CV/metabolic, renal disease). Also, increasing failure rates on current therapies continue to be a problem, due either to the presence or emergence of resistant strains or to non-compliance attributed to drug holidays or adverse side effects. For example, despite therapy, it has been estimated that 63% of subjects receiving combination therapy remained viremic, as they had viral loads >500 copies/mL (Oette, M, Kaiser, R, Däumer, M, et al. Primary HIV Drug Resistance and Efficacy of First-Line Antiretroviral Therapy Guided by Resistance Testing. J Acq Imm Def Synd 2006; 41(5):573-581). Among these patients, 76% had viruses that were resistant to one or more classes of antiretroviral agents. As a result, new drugs are needed that are easier to take, have high genetic barriers to the development of resistance and have improved safety over current agents. In this panoply of choices, novel MOAs that can be used as part of the preferred highly active antiretroviral therapy (HAART) regimen can still have a major role to play since they should be effective against viruses resistant to current agents.

Certain therapeutic compounds are disclosed in WO 2013/006738, WO 2014/110298, and WO 2014/134566.

What is now needed in the art are additional compounds which are novel and useful in the treatment of HIV. Additionally, these compounds may desirably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of Formula I, including pharmaceutically acceptable salts thereof, as well as pharmaceutical compositions, and their use in inhibiting HIV and treating those infected with HIV or AIDS.

In one aspect of the invention, there is provided a compound of Formula I, including pharmaceutically acceptable salts thereof:

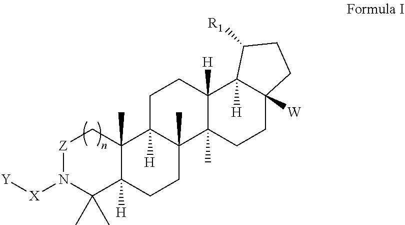

wherein n is 1 or 2;
R₁ is isopropenyl or isopropyl;
X is a phenyl or heteroaryl ring substituted with A, wherein A is at least one member selected from —H, halogen, hydroxyl, —C₁₋₆ alkyl, —C₂₋₆ alkenyl, —C₁₋₆ alkoxy, and —COOR₂;

alternatively, X is selected from a single bond, —C₁₋₆alkyl-, —C₁₋₆alkylaryl-, —C₂₋₆alkenyl-, —C₂₋₆alkenylaryl-, —CO—, —SO₂—, —C₁₋₆alkylCO—, —C₂₋₆alkenylCO—, —COalkylsubstituted C₁₋₆ alkyl-, —COspiroalkylsubstitutedC₁₋₆ alkyl-, —COarylsubstitutedC₁₋₆ alkyl-, —COarylsubstituted C₂₋₆ alkenyl-, —COheteroaryl-, —COaryl-, —COC₁₋₆ alkylaryl-, —COC₁₋₆ alkylheteroaryl-, —COalkylsubstituted C₃₋₆ cycloalkyl-, —COC₂₋₆ alkenylaryl-, —COC₁₋₆ alkyl(NHR₀)—, —C₁₋₆ alkyl(CONHR₀)—, —(COCO)NR₀SO₂—, —SO₂C₁₋₆ alkyl-, —SO₂alkylsubstitutedC₁₋₆ alkyl-, —SO₂arylsubstituted C₁₋₆ alkyl-, —SO₂C₁₋₆ alkylaryl-, —SO₂C₂₋₆ alkenylaryl-, —SO₂aryl-, —SO₂arylC₁₋₆alkyl-, —SO₂arylC₂₋₆alkenyl-, —SO₂heteroaryl-, -heteroaryl-Q₀- and -aryl-Q₀-;

Q₀ is selected from —C₁₋₆ alkyl, -halo, —CF₃ and —OC₁₋₆ alkyl,

R₀ is H, —C₁₋₆ alkyl, -alkylsubstituted C₁₋₆ alkyl, -alkylsubstituted(C₁₋₆)COOR₆, -spiroalkylsubstituted(C₁₋₆)COOR₆, or -arylsubstituted C₁₋₆ alkyl;
R₂ is —H, —C₁₋₆ alkyl, -alkylsubstituted C₁₋₆ alkyl, or-arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₚ—COOR₂, —SO₂NR₂C(O)R₂, -tetrazole, and —CONHOH, wherein p is 1-6;
alternatively, Y is selected from a phenyl or heteroaryl ring, optionally further substituted with 1 to 3 substituents selected from —H, -halo, -hydroxyl, —C₁₋₆ alkyl, —C₁₋₆ alkoxy, —COOR₂, —CN, —NO₂, —CF₃, —SO₂, —NR₂₆R₂₇, —CONR₂₆R₂₇, and —SO₂NR₂₆R₂₇;
alternatively, —X-Y is selected from

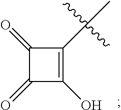 [SMILES: *c1c(O)c(=O)c1=O]

R₃ is —C₁₋₆ alkyl or -alkylsubstituted C₁₋₆ alkyl;
Z is selected from —CO— and —CH₂—;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, and —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, halogen, —CF₃, —OR₂, —COOR₂, —NR₈R₉, —CONR₁₀R₁₁ and —SO₂R₇;
alternatively, R₄ is selected from —C₃₋₆ cycloalkyl, —C₁₋₆ substituted alkyl, —C₁₋₆ alkyl-heteroaryl, —C₁₋₆ alkyl-substitutedheteroaryl, —C₁₋₆ alkyl-NR₆R₇, —C₁₋₆ alkyl-CONR₈R₉, —C₃₋₆ cycloalkyl-CONR₈R₉, —C₃₋₆ cycloalkyl-(CH₂)₁₋₃—NR₆R₇, —(CH₂)₁₋₃—C₃₋₆ cycloalkyl-NR₆R₇; —(CH₂)₁₋₃—C₃₋₆ cycloalkyl-(CH₂)₁₋₃—NR₆R₇; —C₁₋₆ alkyl-Q′₁, C₃₋₆ cycloalkyl-Q₁, —COR₁₀, —SO₂R₃, and

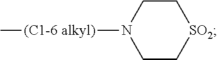

wherein Q′₁ is selected from-hydroxy, —COOR₂, -halo, and —SO₂Rₐ;
Rₐ is C₁₋₆ alkyl, NR₂R₂,

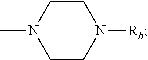

R_{b} is —H, —C₁₋₆ alkyl, —COR₃, —SO₂R₃, —SONR₃R₃;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;

R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
alternatively R₈ and R₉ are taken together with the adjacent N to form a cycle selected from:

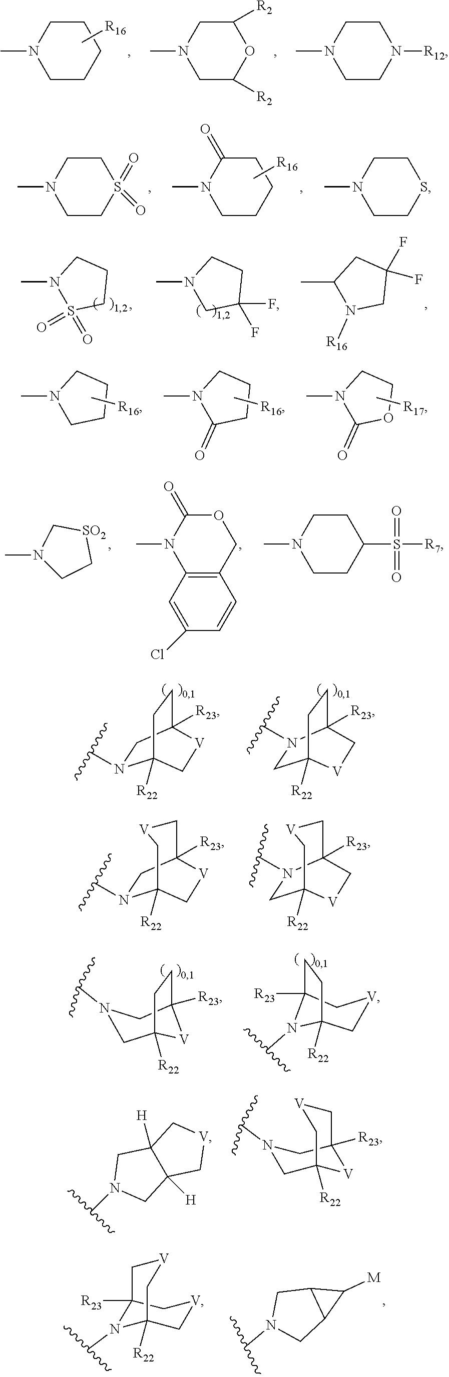

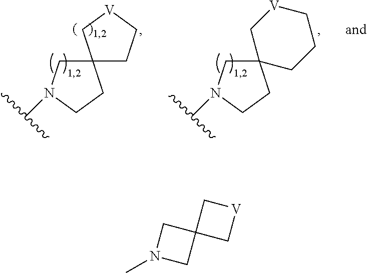

V is selected from —CR₂₄R₂₅—, —SO₂—, —O— and —NR₁₂—;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
alternatively R₁₀ and R₁₁ are taken together with the adjacent N to form a cycle such as

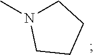

R₁₂ is selected from —C₁₋₆ alkyl, —C₁₋₆ alkyl-OH; —C₁₋₆ alkyl, —C₁₋₆ substituted alkyl,-C₃₋₆ cycloalkyl, —COR, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
alternatively R₁₃ and R₁₄ are taken together with the adjacent N to form a cycle selected from:

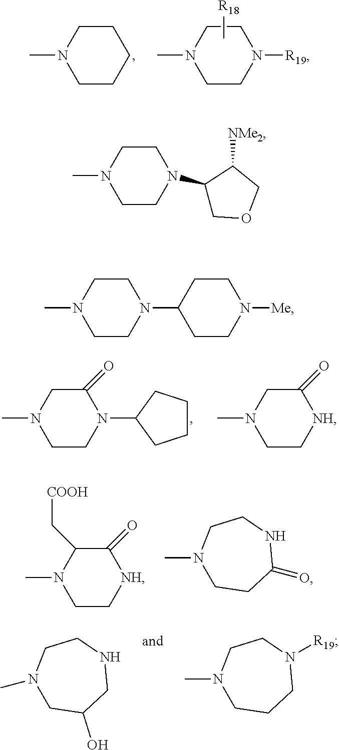

Q₃ is selected from heteroaryl, substituted heteroaryl, —NR₂₀R₂₁, —CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃, wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
alternatively R₂₀ and R₂₁ are taken together with the adjacent N to form a cycle selected from

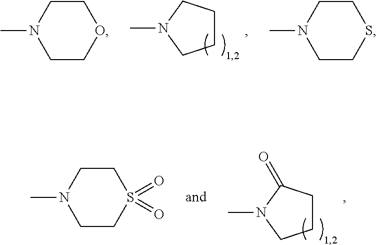

with the proviso that only one of R₂₀ or R₂₁ can be —COR₃;
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from

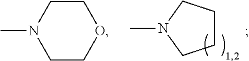

R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂, and

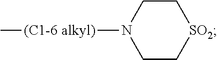

or alternatively R₂₆ and R₂₇ are taken together with the adjacent N to form a cycle selected from:

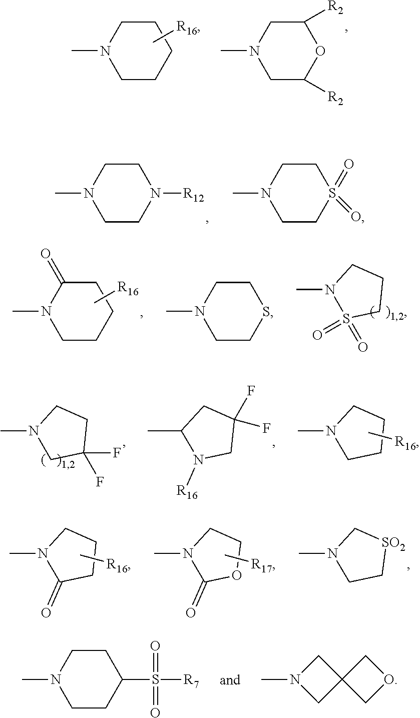

With respect to the compounds of of the present invention, “aza-substituted” means that the compounds contain a six-membered (n=1) or seven-membered (n=2) nitrogen-containing ring. This nitrogen-containing ring is depicted at the lower lefthand portion of the structure of Formula I, Formula II, and Formula III. This nitrogen-containing ring corresponds to the “A” ring (per THE IUPAC convention) of terpenes and triterpenoids as described further herein.

In an aspect of the invention, there is provided a composition useful for treating HIV infection comprising a therapeutic amount of a compound of Formula I, II or III and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, II or III or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I, II or III.

Also provided as part of the invention are one or more methods for making the compounds of the invention.

The present invention is directed to these, as well as other important ends, hereinafter described.

### DETAILED DESCRIPTION OF THE INVENTION

The singular forms “a”, “an”, and “the” include plural reference unless the context dictates otherwise.

Unless otherwise expressly set forth elsewhere in the application, the following terms shall have the following meanings:

“Alkenyl” means a straight or branched alkyl group comprised of 2 to 10 carbons with at least one double bond and optionally substituted with 0-3 halo or alkoxy group.

“Alkenyloxy” means an alkenyl group attached to the parent structure by an oxygen atom.

“Alkoxy” means an alkyl group attached to the parent structure by an oxygen atom.

“Alkoxycarbonyl” means an alkoxy group attached to the parent structure by a carbonyl moiety.

“Alkyl” means a straight or branched saturated hydrocarbon comprised of 1 to 10 carbons, and preferably 1 to 6 carbons.

“Alkylthioxy” means an alkyl group attached to the parent structure through a sulfur atom.

“Alkynyl” means a straight or branched alkyl group comprised of 2 to 10 carbons, preferably 2 to 6 carbons, containing at least one triple bond and optionally substituted with 0-3 halo or alkoxy group.

“Aryl” mean a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic group include, but are not limited to, phenyl, biphenyl, cyclopropylphenyl, indane, naphthalene, and tetrahydronaphthalene. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n is 1-5, —CH(CH₃)Ph, —CH(Ph)₂.

“Aryloxy” is an aryl group attached to the parent structure by oxygen.

“Azaindole” means one of the “CH” moieties in the 6-member ring of an indole is substituted with a nitrogen atom.

“Azaindoline” means one of the aromatic “CH” moieties of an indoline is substituted with a nitrogen atom.

“Azatetrahydroquinoline” means any aromatic CH moiety of tetrahydroquinoline is substituted with a nitrogen atom.

“Benzyloxy” means a benzyl group is attached to the parent structure through an oxygen atom. The phenyl group of the benzyl moiety could be optionally substituted by 1-3 moieties independently selected from alkyl, alkoxy, halo, haloalkyl, haloalkoxy and cyano. “Cₓ-C₇” notation indicates a structural element comprised of carbons numbering between ‘x’ and ‘y’. For example, “C₅-C₁₀ bicycloalkyl” means a bicyclic ring system comprised of 5 to 10 carbons, where the rings are attached in a fused, spiro or bridged manner; an example of C₅-C₁₀ bicycloalkyl include, but is not limited to, bicyclo[2.2.2]octane. Similarly, “C₃-C₄ cycloalkyl” is a subset of monocyclic ring system comprised of 3 to 4 carbons.

“Cycloalkyl” means a monocyclic ring system comprised of 3 to 7 carbons.

“Cyano” refers to —CN.

“Diazaindole” means any two “CH” moieties in the 6-member ring of an indole are substituted with nitrogen atoms.

“Diazaindoline” means any two aromatic “CH” moieties of an indoline are substituted with a nitrogen atom.

“Diazatetrahydroquinoline” means any two aromatic CH moieties of tetrahydroquinoline are substituted with nitrogen atoms.

“Halo” or “halogen” refers to —F, —Cl, —Br, or —I.

“Haloalkyl” means an alkyl group substituted by any combination of one to six halogen atoms.

“Haloalkoxy” or “Haloalkyloxy” means a haloalkyl group attached to the parent structure through an oxygen atom.

“Hydroxy” refers to —OH.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from oxygen, nitrogen and sulfur. The rings could be bridged, fused and/or bonded, through a direct or spiro attachment, with the option to have one or a combination thereof be aromatic. Examples include, but are not limited to, azaindole, azaindoline, azetidine, benzimidazole, benzodioxolyl, benzoisothiazole, benzothiazole, benzothiadiazole, benzothiophene, benzoxazole, carbazole, chroman, dihalobezodioxolyl, dihydrobenzofuran, dihydrobenzo[1,4]oxazine, 1,3-dihydrobenzo[c]thiophene 2,2-dioxide, 2,3-dihydrobenzo[d]isothiazole 1,1-dioxide, 3,4-dihydro-2H-pyrido[3,2-b][1,4]oxazine, 2,3-dihydro-1H-pyrrolo[3,4-c]pyridine and its regioisomeric variants, 6,7-dihydro-5H-pyrrolo[2,3-b]pyrazine and its regioisomeric variants, furanylphenyl, imidazole, imidazo[1,2-a]pyridine, indazole, indole, indoline, isoquinoline, isoquinolinone, isothiazolidine 1,1-dioxide, morpholine, 2-oxa-5-azabicyclo[2.2.1]heptane, oxadiazole-phenyl, oxazole, phenylaztidine, phenylindazole, phenylpiperidine, phenylpiperizine, phenyloxazole, phenylpyrrolidine, piperidine, pyridine, pyridinylphenyl, pyridinylpyrrolidine, pyrimidine, pyrimidinylphenyl, pyrrazole-phenyl, pyrrolidine, pyrrolidin-2-one, 1H-pyrazolo[4,3-c]pyridine and its regioisomeric variants, pyrrole, 5H-pyrrolo[2,3-b]pyrazine, 7H-pyrrolo[2,3-d]pyrimidine and its regioisomeric variants, quinazoline, quinoline, quinoxaline, tetrahydroisoquinoline, 1,2,3,4-tetrahydro-1,8-naphthyridine, tetrahydroquinoline, 4,5,6,7-tetrahydrothieno[3,2-c]pyridine, 1,2,5-thiadiazolidine 1,1-dioxide, thiophene, thiophenylphenyl, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine. In addition the “regioisomer variants” notation as in, for example, “5H-pyrrolo[2,3-b]pyrazine and its regioisomeric variants” would also encompass 7H-pyrrolo[2,3-d]pyrimidine, 7H-pyrrolo[2,3-c]pyridazine, 1H-pyrrolo[2,3-d]pyridazine, 5H-pyrrolo[3,2-c]pyridazine, and 5H-pyrrolo[3,2-d]pyrimidine. Similarly, 6,7-dihydro-5H-pyrrolo[2,3-b]pyrazine and its regioisomeric variants would encompass 6,7-dihydro-5H-pyrrolo[2,3-d]pyrimidine and 6,7-dihydro-5H-pyrrolo[2,3-c]pyridazine. It is also understood that the lack of “regioisomeric variants” notation does not in any way restrict the claim scope to the noted example only.

“Heterocyclylalkyl” is a heterocyclyl moiety attached to the parent structure through C₁-C₅ alkyl group. Examples include, but are not limited to, —(CH₂)ₙ—R^{Z} or —CH(CH₃)—(R^{Z}) where n is 1-5 and that R^{Z} is chosen from benzimidazole, imidazole, indazole, isooxazole, phenyl-pyrazole, pyridine, quinoline, thiazole, triazole, triazolone, oxadiazole.

“Triterpene” or “triterpenoid” means a class of compounds based on three terpene units, which are in turn each based on two isoprene units. Triterpenes exist in a large variety of structures and can be broadly divided according to the number of rings present. The aza-substituted triterpenoids of the present invention are in general pentacyclic structures, i.e. having five rings, with one of these rings (the “A” ring as per the IUPAC naming convention) being nitrogen-substituted, as per Formula I. The following structure of cholesterol (a 4-ring terpenoid) illustrates the IUPAC ring naming convention.

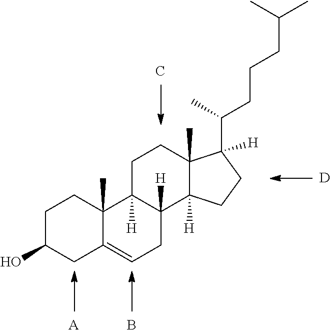

### Cholesterol and IUPAC Ring Naming Convention

“Tetrahydroquinoline” means 1,2,3,4-tetrahydroquinoline.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R.

Also, the “X” substituent which is connected to the “Y” substituent and the nitrogen of the aza ring, as in e.g., Formula I,

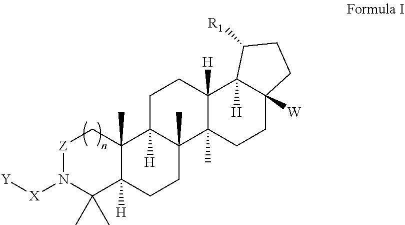

is understood that it can be connected in either direction, unless indicated otherwise. For example, “X” being selected from —C₁₋₆alkylaryl-, means that the substituent, as illustrated for C₁₋₆alkyl being (CH₂)₆ and aryl being phenyl, can be connected in either orientation as shown immediately below.

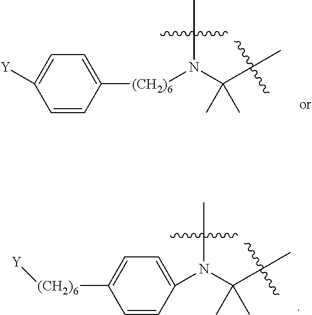

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereomers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

For the sake of efficiency some ring structures are shown with a variable number of members in the ring. For example, the following ring substituent

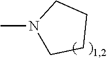

having the parenthetical “( )_{1,2}” is intended to encompass both a single carbon group, —(CH₂)—, and a two carbon group, —(CH₂CH₂)—. The intended ring structures could individually be depicted as:

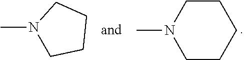

The compounds of the invention also include “prodrugs”. The term “prodrug” as used herein encompasses both the term “prodrug esters” and the term “prodrug ethers”.

The common numbering system for the general core of the pentacyclic triterpenoid compounds of the present invention is shown below for an illustrative triterpene: betulin. This numbering system is in accordance with IUPAC rules.

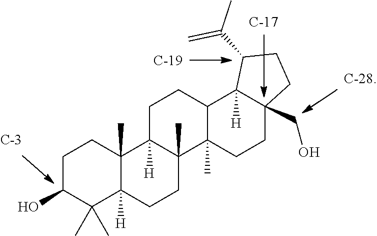

In an aspect of the invention, there is provided a compound of Formula I, including pharmaceutically acceptable salts thereof:

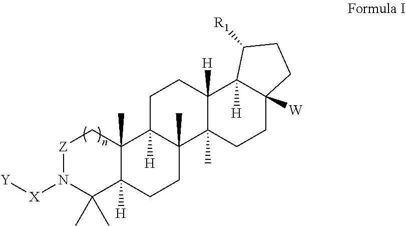

wherein n is 1 or 2;
R₁ is isopropenyl or isopropyl;
X is a phenyl or heteroaryl ring substituted with A, wherein A is at least one member selected from —H, halogen, hydroxyl, —C₁₋₆ alkyl, —C₂₋₆ alkenyl, —C₁₋₆ alkoxy, and —COOR₂;
alternatively, X is selected from a single bond, —C₁₋₆alkyl-, —C₁₋₆alkylaryl-, —C₂₋₆alkenyl-, —C₂₋₆alkenylaryl-, —CO—, —SO₂—, —C₁₋₆alkylCO—, —C₂₋₆alkenylCO—, —COalkylsubstituted C₁₋₆ alkyl-, —COspiroalkylsubstitutedC₁₋₆ alkyl-, —COarylsubstitutedC₁₋₆ alkyl-, —COarylsubstituted C₂₋₆ alkenyl-, —COheteroaryl-, —COaryl-, —COC₁₋₆ alkylaryl-, —COC₁₋₆ alkylheteroaryl-, —COalkylsubstituted C₃₋₆ cycloalkyl-, —COC₂₋₆ alkenylaryl-, —COC₁₋₆ alkyl(NHR₀)—, —C₁₋₆ alkyl(CONHR₀)—, —(COCO)NR₀SO₂—, —SO₂C₁₋₆ alkyl-, —SO₂alkylsubstitutedC₁₋₆ alkyl-, —SO₂arylsubstituted C₁₋₆ alkyl-, —SO₂C₁₋₆ alkylaryl-, —SO₂C₂₋₆ alkenylaryl-, —SO₂aryl-, —SO₂arylC₁₋₆alkyl-, —SO₂arylC₂₋₆alkenyl-, —SO₂heteroaryl-, -heteroaryl-Q₀- and -aryl-Q₀-;
Q₀ is selected from —C₁₋₆ alkyl, -halo, —CF₃ and —OC₁₋₆ alkyl,
R₀ is H, —C₁₋₆ alkyl, -alkylsubstituted C₁₋₆ alkyl, -alkylsubstituted(C₁₋₆)COOR₆, -spiroalkylsubstituted(C₁₋₆)COOR₆, or -arylsubstituted C₁₋₆ alkyl;
R₂ is —H, —C₁₋₆ alkyl, -alkylsubstituted C₁₋₆ alkyl, or-arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₚ—COOR₂, —SO₂NR₂C(O)R₂, -tetrazole, and —CONHOH, wherein p is 1-6;
alternatively, Y is selected from a phenyl or heteroaryl ring, optionally further substituted with 1 to 3 substituents selected from —H, -halo, -hydroxyl, —C₁₋₆ alkyl, —C₁₋₆ alkoxy, —COOR₂, —CN, —NO₂, —CF₃, —SO₂, —NR₂₆R₂₇, —CONR₂₆R₂₇, and —SO₂NR₂₆R₂₇;
alternatively, —X-Y is selected from

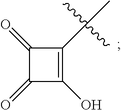 [SMILES: *c1c(O)c(=O)c1=O]

R₃ is —C₁₋₆ alkyl or -alkylsubstituted C₁₋₆ alkyl;
Z is selected from —CO— and —CH₂—;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, and —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, halogen, —CF₃, —OR₂, —COOR₂, —NR₈R₉, —CONR₁₀R₁₁ and —SO₂R₇;
alternatively, R₄ is selected from —C₃₋₆ cycloalkyl, —C₁₋₆ substituted alkyl, —C₁₋₆ alkyl-heteroaryl, —C₁₋₆ alkyl-substitutedheteroaryl, —C₁₋₆ alkyl-NR₆R₇, —C₁₋₆ alkyl-CONR₈R₉, —C₃₋₆ cycloalkyl-CONR₈R₉, —C₃₋₆ cycloalkyl-(CH₂)₁₋₃—NR₆R₇, —(CH₂)₁₋₃—C₃₋₆ cycloalkyl-NR₆R₇; —(CH₂)₁₋₃—C₃₋₆ cycloalkyl-(CH₂)₁₋₃—NR₆R₇; —C₁₋₆ alkyl-Q′₁, C₃₋₆ cycloalkyl-Q₁, —COR₁₀, —SO₂R₃, and

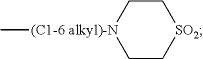

wherein Q′₁ is selected from-hydroxy, —COOR₂, -halo, and —SO₂Rₐ;
Rₐ is C₁₋₆ alkyl, NR₂R₂,

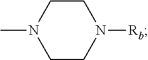

R_{b} is —H, —C₁₋₆ alkyl, —COR₃, —SO₂R₃, —SONR₃R₃;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
alternatively R₈ and R₉ are taken together with the adjacent N to form a cycle selected from:

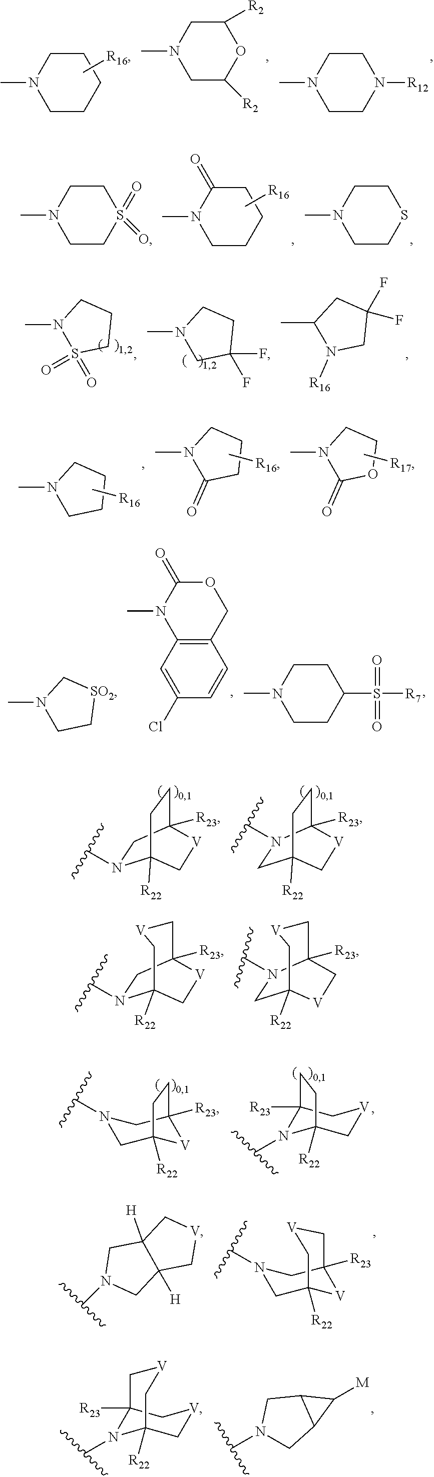

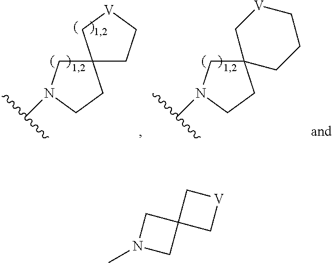

V is selected from —CR₂₄R₂₅—, —SO₂—, —O— and —NR₁₂—;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
alternatively R₁₀ and R₁₁ are taken together with the adjacent N to form a cycle such as

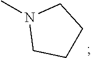

R₁₂ is selected from —C₁₋₆ alkyl, —C₁₋₆ alkyl-OH; —C₁₋₆ alkyl, —C₁₋₆ substituted alkyl,-C₃₋₆ cycloalkyl, —COR, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
alternatively R₁₃ and R₁₄ are taken together with the adjacent N to form a cycle selected from:

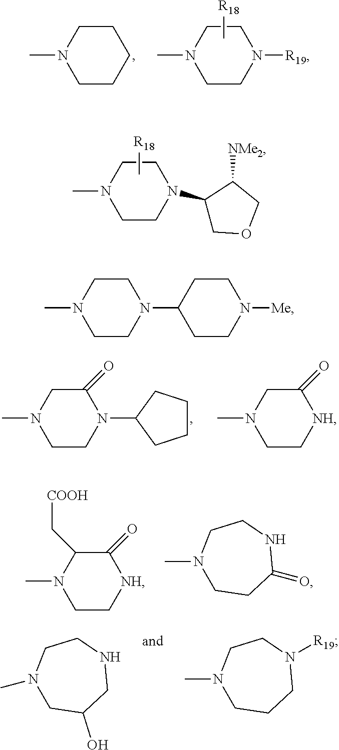

Q₃ is selected from heteroaryl, substituted heteroaryl, —NR₂₀R₂₁, —CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃, wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
alternatively R₂₀ and R₂₁ are taken together with the adjacent N to form a cycle selected from

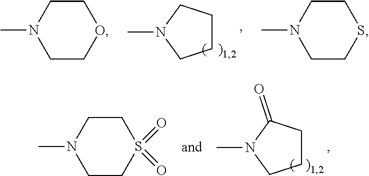

with the proviso that only one of R₂₀ or R₂₁ can be —COR₃;
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from

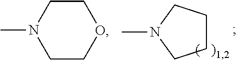

R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂, and

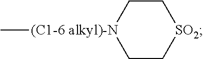

or alternatively R₂₆ and R₂₇ are taken together with the adjacent N to form a cycle selected from:

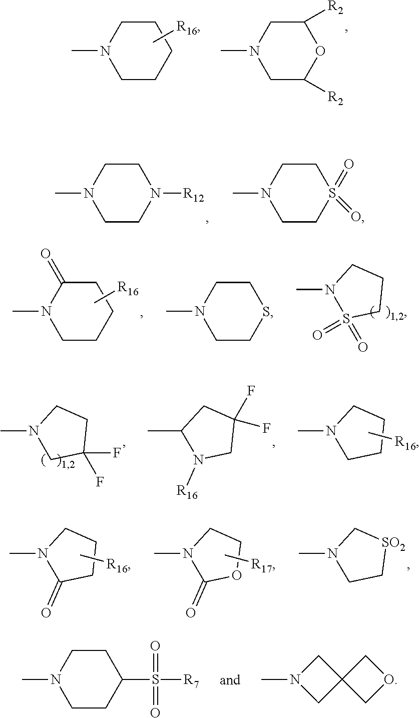

In an aspect of the invention, there is provided a compound of Formula I, wherein R₁ is isopropenyl.

In an aspect of the invention, there is provided a compound of Formula I, wherein Z is —CH₂—.

In an aspect of the invention, there is provided a compound of Formula I, wherein n is 1.

In an aspect of the invention, there is provided a compound of Formula I, wherein n is 2.

In an aspect of the invention, there is provided a compound of Formula I, wherein X is selected from phenyl, pyridyl, pyrimidinyl, pyrazinyl, and pyradazinyl;

In an aspect of the invention, there is provided a compound of Formula I, wherein X is selected from phenyl, pyridyl, and pyrimidinyl;

In an aspect of the invention, there is provided a compound of Formula I, wherein X is phenyl.

In an aspect of the invention, there is provided a compound of Formula I, wherein Y is —COOR₂.

In an aspect of the invention, there is provided a compound of Formula I, wherein Y is —COOH.

In an aspect of the invention, there is provided a compound of Formula I, wherein A is —H.

In an aspect of the invention, there is provided a compound of Formula I, wherein X is selected from —CO—, —COC₁₋₆alkyl-, —C₁₋₆alkylCO—, —COaryl-, —COheteroaryl-, —SO₂aryl-, and —SO₂heteroaryl-.

In an aspect of the invention, there is provided a compound of Formula I, wherein R₄ is selected from —H, —C₁₋₆ alkyl, —C₁₋₆ alkyl-Q₁, and —COR₆.

In an aspect of the invention, there is provided a compound of Formula I, wherein —R₅ is —H.

In an aspect of the invention, there is provided a compound of Formula I, wherein R₄ is —C₁₋₆ alkyl-Q₁.

In an aspect of the invention, there is provided a compound of Formula I, wherein Q₁ is —NR₈R₉.

In an aspect of the invention, there is provided a compound of Formula I, wherein R₄ is —COR₆.

In an aspect of the invention, there is provided a compound of Formula I, wherein W is —CH₂OR₂

In an aspect of the invention, there is provided a compound of Formula I, wherein R₂ is H.

In an aspect of the invention, there is provided a compound of Formula I, wherein W is —COOR₂.

In an aspect of the invention, there is provided a compound of Formula I, wherein W is —COOH.

In an aspect of the invention, there is provided a compound of Formula I, wherein W is —NR₄R₅.

In an aspect of the invention, there is provided a compound of Formula I, wherein W is —CONR₂₆R₂₇.

In an aspect of the invention, there is provided a compound of Formula I, wherein W is —CH₂NR₂₆R₂₇.

In an aspect of the invention, there is provided a compound of Formula I, wherein W is —NR₄COR₆.

In an aspect of the invention, there is provided a compound of Formula I, wherein W is —NR₄C(O)NR₄R₅.

In an aspect of the invention, there is provided a compound of Formula I, wherein W is —NR₄COOR₆.

In an aspect of the invention, there is provided a compound of Formula II, including pharmaceutically acceptable salts thereof:

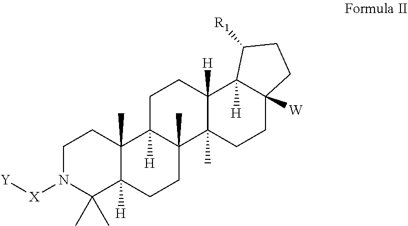

wherein R₁ is isopropenyl or isopropyl;
X is a phenyl or heteroaryl ring substituted with A, wherein A is at least one member selected from —H, halogen, hydroxyl, —C₁₋₆ alkyl, —C₂₋₆ alkenyl, —C₁₋₆ alkoxy, and —COOR₂;
alternatively, X is selected from a single bond, —C₁₋₆alkyl-, —C₁₋₆alkylaryl-, —C₂₋₆alkenyl-, —C₂₋₆alkenylaryl-, —CO—, —SO₂—, —C₁₋₆alkylCO—, —C₂₋₆alkenylCO—, —COalkylsubstituted C₁₋₆ alkyl-, —COspiroalkylsubstitutedC₁₋₆ alkyl-, —COarylsubstitutedC₁₋₆ alkyl-, —COarylsubstituted C₂₋₆ alkenyl-, —COheteroaryl-, —COaryl-, —COC₁₋₆ alkylaryl-, —COC₁₋₆ alkylheteroaryl-, —COalkylsubstituted C₃₋₆ cycloalkyl-, —COC₂₋₆ alkenylaryl-, —COC₁₋₆ alkyl(NHR₀)—, —C₁₋₆ alkyl(CONHR₀)—, —(COCO)NR₀SO₂—, —SO₂C₁₋₆ alkyl-, —SO₂alkylsubstitutedC₁₋₆ alkyl-, —SO₂arylsubstituted C₁₋₆ alkyl-, —SO₂C₁₋₆ alkylaryl-, —SO₂C₂₋₆ alkenylaryl-, —SO₂aryl-, —SO₂arylC₁₋₆alkyl-, —SO₂arylC₂₋₆alkenyl-, —SO₂heteroaryl-, -heteroaryl-Q₀- and -aryl-Q₀-;
Q₀ is selected from —C₁₋₆ alkyl, -halo, —CF₃ and —OC₁₋₆ alkyl,
R₀ is H, —C₁₋₆ alkyl, -alkylsubstituted C₁₋₆ alkyl, -alkylsubstituted(C₁₋₆)COOR₆, -spiroalkylsubstituted(C₁₋₆)COOR₆, or -arylsubstituted C₁₋₆ alkyl;
R₂ is —H, —C₁₋₆ alkyl, -alkylsubstituted C₁₋₆ alkyl, or-arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₚ—COOR₂, —SO₂NR₂C(O)R₂, -tetrazole, and —CONHOH, wherein p is 1-6;
alternatively, Y is selected from a phenyl or heteroaryl ring, optionally further substituted with 1 to 3 substituents selected from —H, -halo, -hydroxyl, —C₁₋₆ alkyl, —C₁₋₆ alkoxy, —COOR₂, —CN, —NO₂, —CF₃, —SO₂, —NR₂₆R₂₇, —CONR₂₆R₂₇, and —SO₂NR₂₆R₂₇;
alternatively, —X-Y is selected from

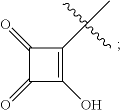 [SMILES: *c1c(O)c(=O)c1=O]

R₃ is —C₁₋₆ alkyl or -alkylsubstituted C₁₋₆ alkyl;
Z is selected from —CO— and —CH₂—;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, and —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, halogen, —CF₃, —OR₂, —COOR₂, —NR₈R₉, —CONR₁₀R₁₁ and —SO₂R₇;
alternatively, R₄ is selected from —C₃₋₆ cycloalkyl, —C₁₋₆ substituted alkyl, —C₁₋₆ alkyl-heteroaryl, —C₁₋₆ alkyl-substitutedheteroaryl, —C₁₋₆ alkyl-NR₆R₇, —C₁₋₆ alkyl-CONR₈R₉, —C₃₋₆ cycloalkyl-CONR₈R₉, —C₃₋₆ cycloalkyl-(CH₂) i-3-NR₆R₇, —(CH₂)₁₋₃—C₃₋₆ cycloalkyl-NR₆R₇; —(CH₂)₁₋₃—C₃₋₆ cycloalkyl-(CH₂)₁₋₃—NR₆R₇; —C₁₋₆ alkyl-Q′₁, C₃₋₆ cycloalkyl-Q₁, —COR₁₀, —SO₂R₃, and

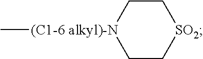

wherein Q′₁ is selected from-hydroxy, —COOR₂, -halo, and —SO₂Rₐ;
Rₐ is C₁₋₆ alkyl, NR₂R₂,

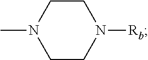

R_{b} is —H, —C₁₋₆ alkyl, —COR₃, —SO₂R₃, —SONR₃R₃;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆alkyl-Q₂, and —COOR₃,
alternatively R₈ and R₉ are taken together with the adjacent N to form a cycle selected from:

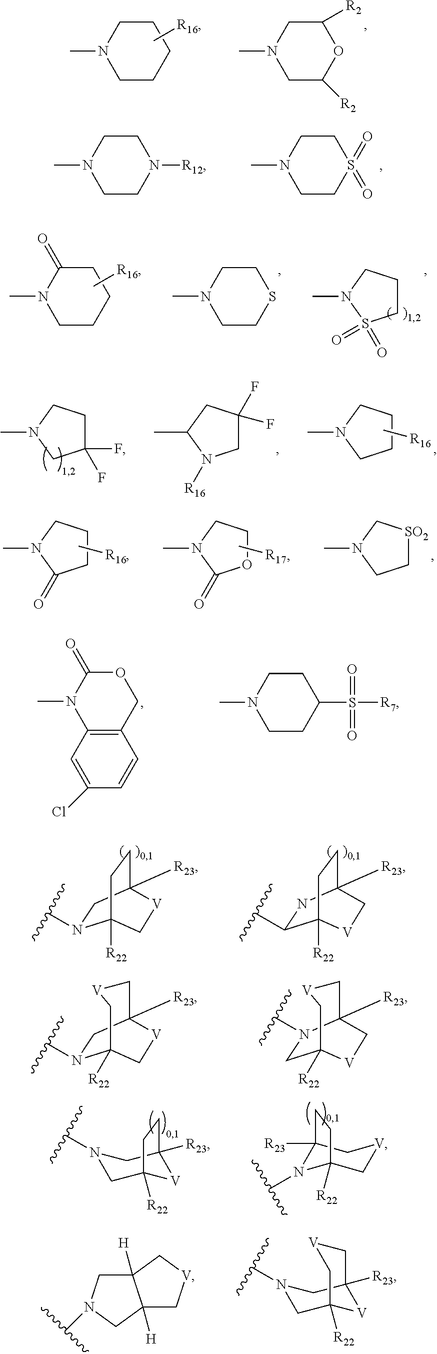

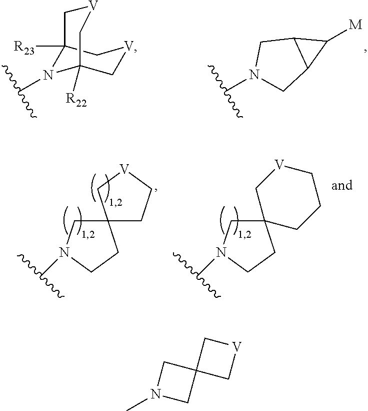

V is selected from —CR₂₄R₂₅—, —O— and —NR₁₂—;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
alternatively R₁₀ and R₁₁ are taken together with the adjacent N to form a cycle such as

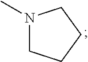

R₁₂ is selected from —C₁₋₆ alkyl, —C₁₋₆ alkyl-OH; —C₁₋₆ alkyl, —C₁₋₆ substituted alkyl,-C₃₋₆ cycloalkyl, —COR, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
alternatively R₁₃ and R₁₄ are taken together with the adjacent N to form a cycle selected from:

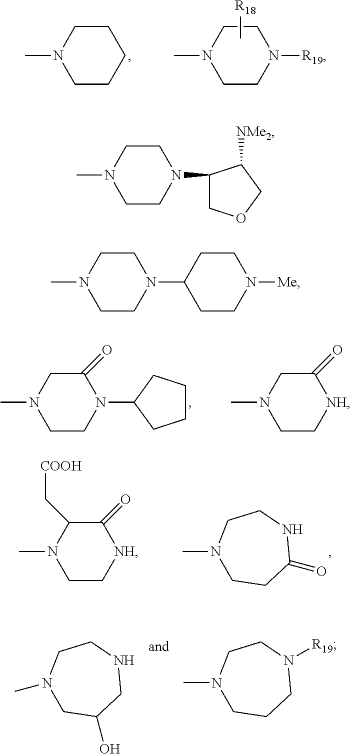

Q₃ is selected from heteroaryl, substituted heteroaryl, —NR₂₀R₂₁, —CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃, wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
alternatively R₂₀ and R₂₁ are taken together with the adjacent N to form a cycle selected from

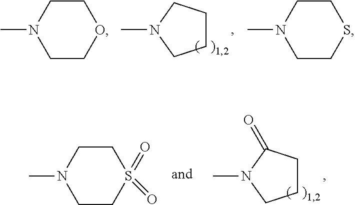

with the proviso that only one of R₂₀ or R₂₁ can be —COR₃,
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from

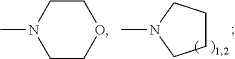

R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂, and

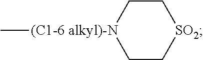

or alternatively R₂₆ and R₂₇ are taken together with the adjacent N to form a cycle selected from:

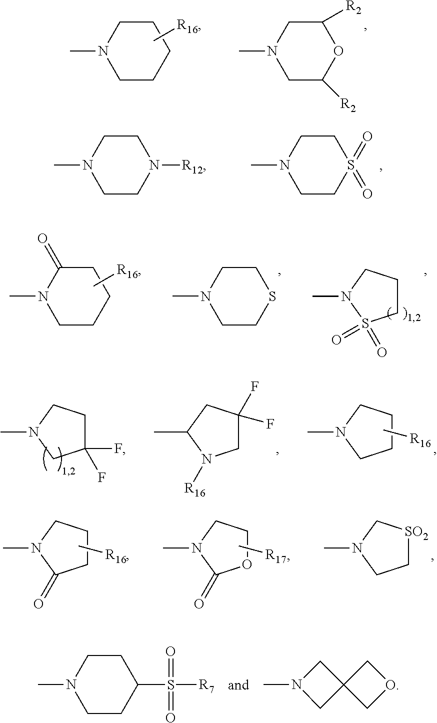

In an aspect of the invention, there is provided a compound of Formula IIII, including pharmaceutically acceptable salts thereof:

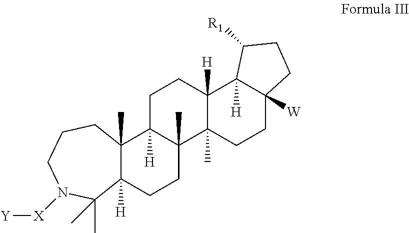

wherein R₁ is isopropenyl or isopropyl;
X is a phenyl or heteroaryl ring substituted with A, wherein A is at least one member selected from —H, halogen, hydroxyl, —C₁₋₆ alkyl, —C₂₋₆ alkenyl, —C₁₋₆ alkoxy, and —COOR₂;
alternatively, X is selected from a single bond, —C₁₋₆alkyl-, —C₁₋₆alkylaryl-, —C₂₋₆alkenyl-, —C₂₋₆alkenylaryl-, —CO—, —SO₂—, —C₁₋₆alkylCO—, —C₂₋₆alkenylCO—, —COalkylsubstituted C₁₋₆ alkyl-, —COspiroalkylsubstitutedC₁₋₆ alkyl-, —COarylsubstitutedC₁₋₆ alkyl-, —COarylsubstituted C₂₋₆ alkenyl-, —COheteroaryl-, —COaryl-, —COC₁₋₆ alkylaryl-, —COC₁₋₆ alkylheteroaryl-, —COalkylsubstituted C₃₋₆ cycloalkyl-, —COC₂₋₆ alkenylaryl-, —COC₁₋₆ alkyl(NHR₀)—, —C₁₋₆ alkyl(CONHR₀)—, —(COCO)NR₀SO₂—, —SO₂C₁₋₆ alkyl-, —SO₂alkylsubstitutedC₁₋₆ alkyl-, —SO₂arylsubstituted C₁₋₆ alkyl-, —SO₂C₁₋₆ alkylaryl-, —SO₂C₂₋₆ alkenylaryl-, —SO₂aryl-, —SO₂arylC₁₋₆alkyl-, —SO₂arylC₂₋₆alkenyl-, —SO₂heteroaryl-, -heteroaryl-Q₀- and -aryl-Q₀-;
Q₀ is selected from —C₁₋₆ alkyl, -halo, —CF₃ and —OC₁₋₆ alkyl,
R₀ is H, —C₁₋₆ alkyl, -alkylsubstituted C₁₋₆ alkyl, -alkylsubstituted(C₁₋₆)COOR₆, -spiroalkylsubstituted(C₁₋₆)COOR₆, or -arylsubstituted C₁₋₆ alkyl;
R₂ is —H, —C₁₋₆ alkyl, -alkylsubstituted C₁₋₆ alkyl, or-arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₚ—COOR₂, —SO₂NR₂C(O)R₂, -tetrazole, and —CONHOH, wherein p is 1-6;
alternatively, Y is selected from a phenyl or heteroaryl ring, optionally further substituted with 1 to 3 substituents selected from —H, -halo, -hydroxyl, —C₁₋₆ alkyl, —C₁₋₆ alkoxy, —COOR₂, —CN, —NO₂, —CF₃, —SO₂, —NR₂₆R₂₇, —CONR₂₆R₂₇, and —SO₂NR₂₆R₂₇;
alternatively, —X-Y is selected from

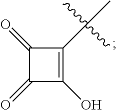 [SMILES: *c1c(O)c(=O)c1=O]

R₃ is —C₁₋₆ alkyl or -alkylsubstituted C₁₋₆ alkyl;
Z is selected from —CO— and —CH₂—;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, and —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, halogen, —CF₃, —OR₂, —COOR₂, —NR₈R₉, —CONR₁₀R₁₁ and —SO₂R₇;
alternatively, R₄ is selected from —C₃₋₆ cycloalkyl, —C₁₋₆ substituted alkyl, —C₁₋₆ alkyl-heteroaryl, —C₁₋₆ alkyl-substitutedheteroaryl, —C₁₋₆ alkyl-NR₆R₇, —C₁₋₆ alkyl-CONR₈R₉, —C₃₋₆ cycloalkyl-CONR₈R₉, —C₃₋₆ cycloalkyl-(CH₂) i-3-NR₆R₇, —(CH₂)₁₋₃—C₃₋₆ cycloalkyl-NR₆R₇; —(CH₂)₁₋₃—C₃₋₆ cycloalkyl-(CH₂)₁₋₃—NR₆R₇; —C₁₋₆ alkyl-Q′₁, C₃₋₆ cycloalkyl-Q₁, —COR₁₀, —SO₂R₃, and

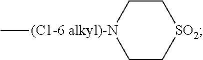

wherein Q′₁ is selected from-hydroxy, —COOR₂, -halo, and —SO₂Rₐ;
Rₐ is C₁₋₆ alkyl, NR₂R₂,

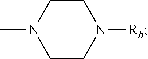

R_{b} is —H, —C₁₋₆ alkyl, —COR₃, —SO₂R₃, —SONR₃R₃;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
alternatively R₈ and R₉ are taken together with the adjacent N to form a cycle selected from:

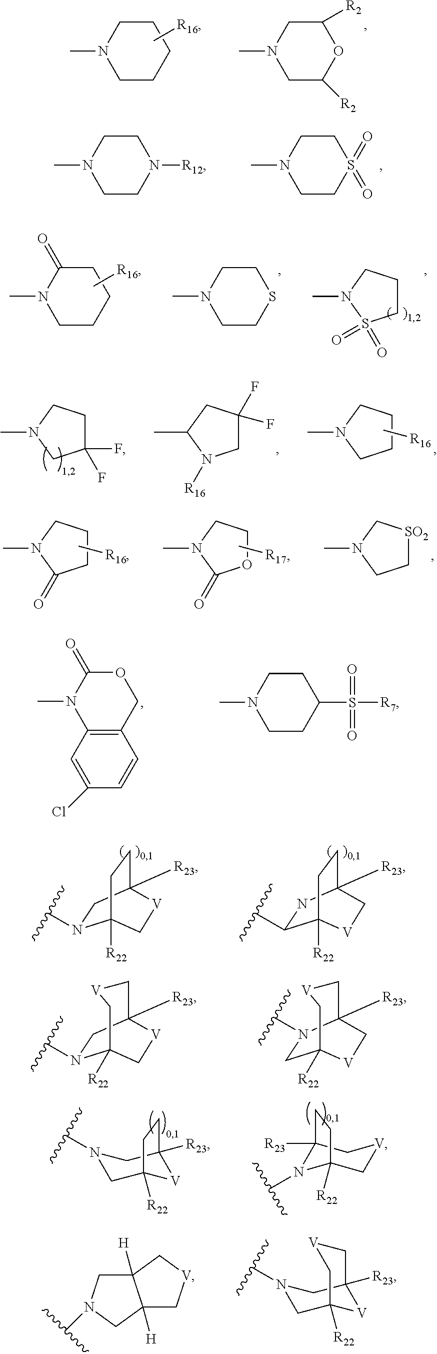

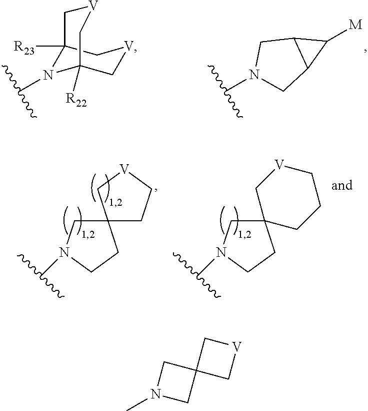

V is selected from —CR₂₄R₂₅—, —O— and —NR₁₂—;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
alternatively R₁₀ and R₁₁ are taken together with the adjacent N to form a cycle such as

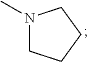

R₁₂ is selected from —C₁₋₆ alkyl, —C₁₋₆ alkyl-OH; —C₁₋₆ alkyl, —C₁₋₆ substituted alkyl,-C₃₋₆ cycloalkyl, —COR, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
alternatively R₁₃ and R₁₄ are taken together with the adjacent N to form a cycle selected from:

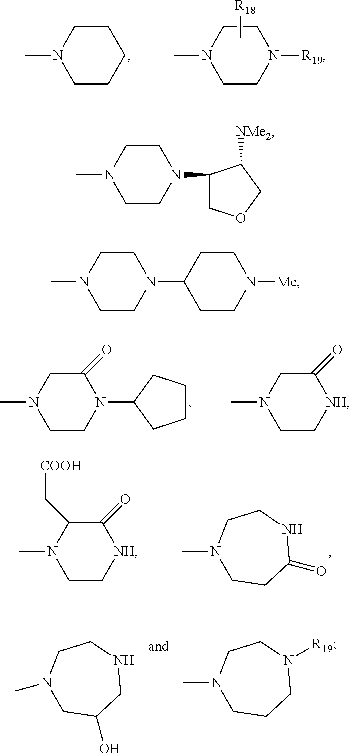

Q₃ is selected from heteroaryl, substituted heteroaryl, —NR₂₀R₂₁, —CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;

R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃, wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
alternatively R₂₀ and R₂₁ are taken together with the adjacent N to form a cycle selected from

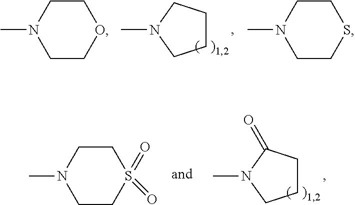

with the proviso that only one of R₂₀ or R₂₁ can be —COR₃,
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from

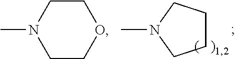

R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂, and

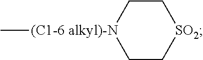

or alternatively R₂₆ and R₂₇ are taken together with the adjacent N to form a cycle selected from:

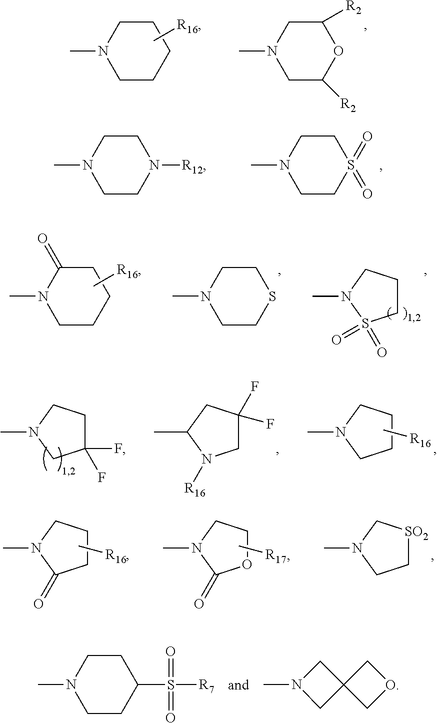

In an aspect of the invention, there is provided a compound, including pharmaceutically acceptable salts thereof, which is selected from:

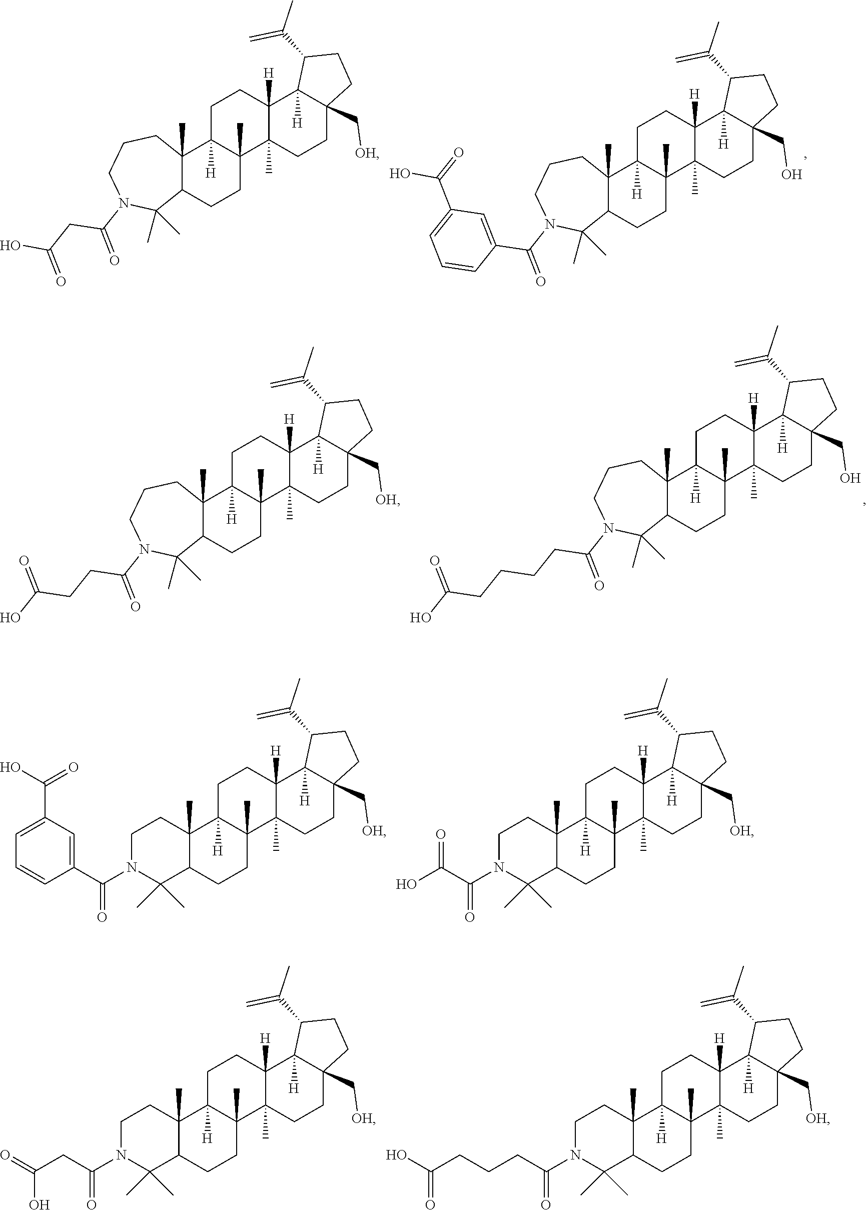

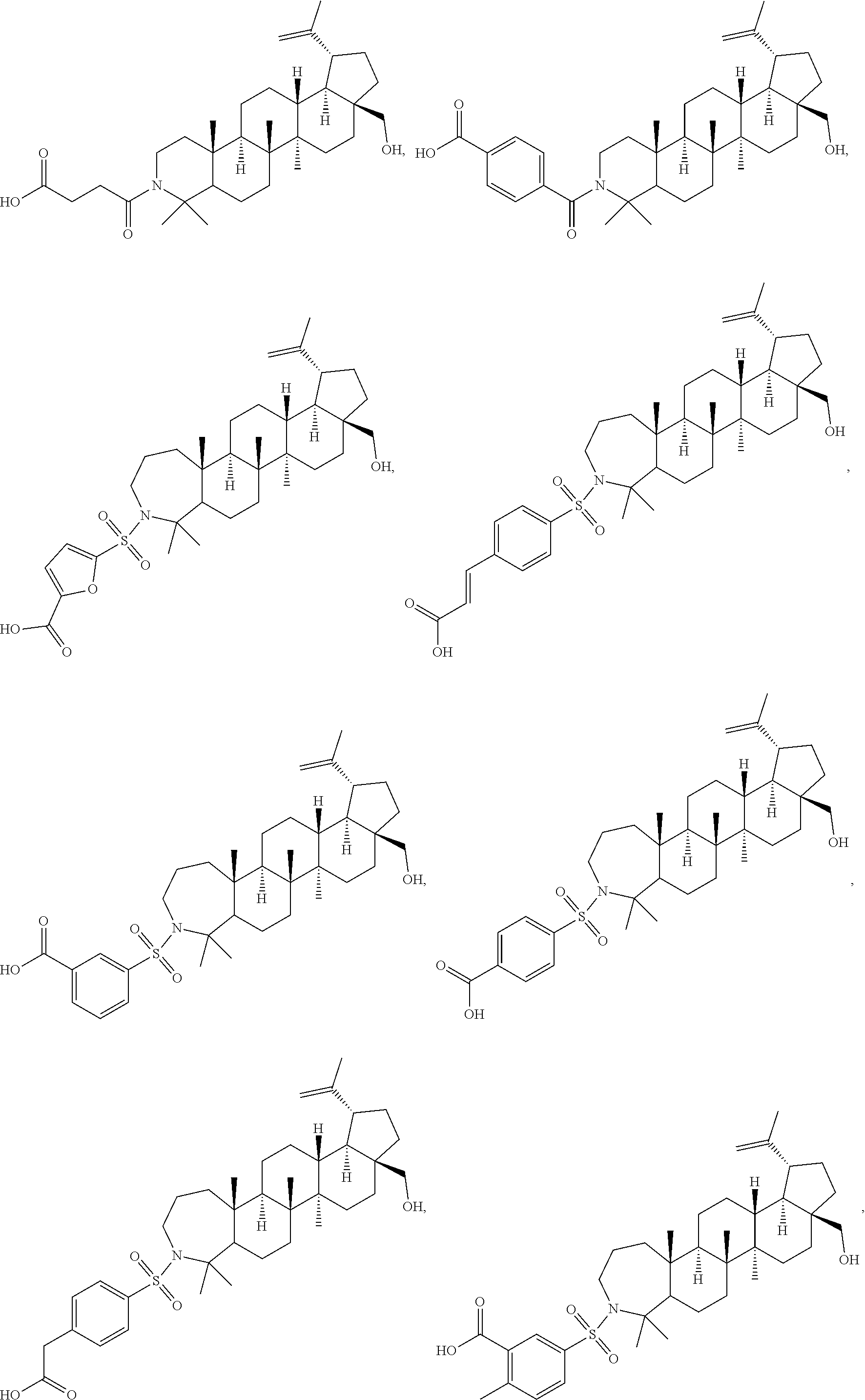

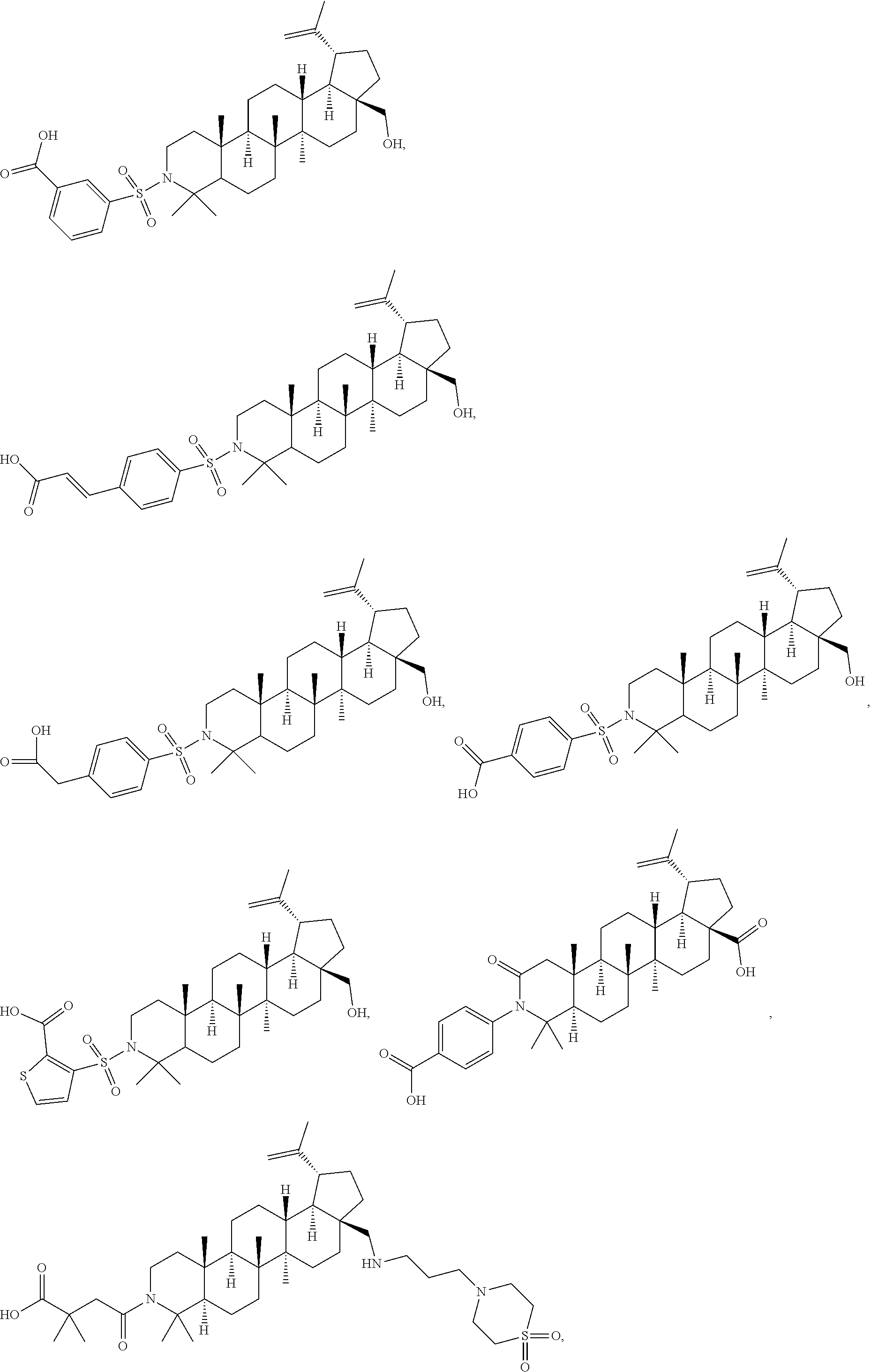

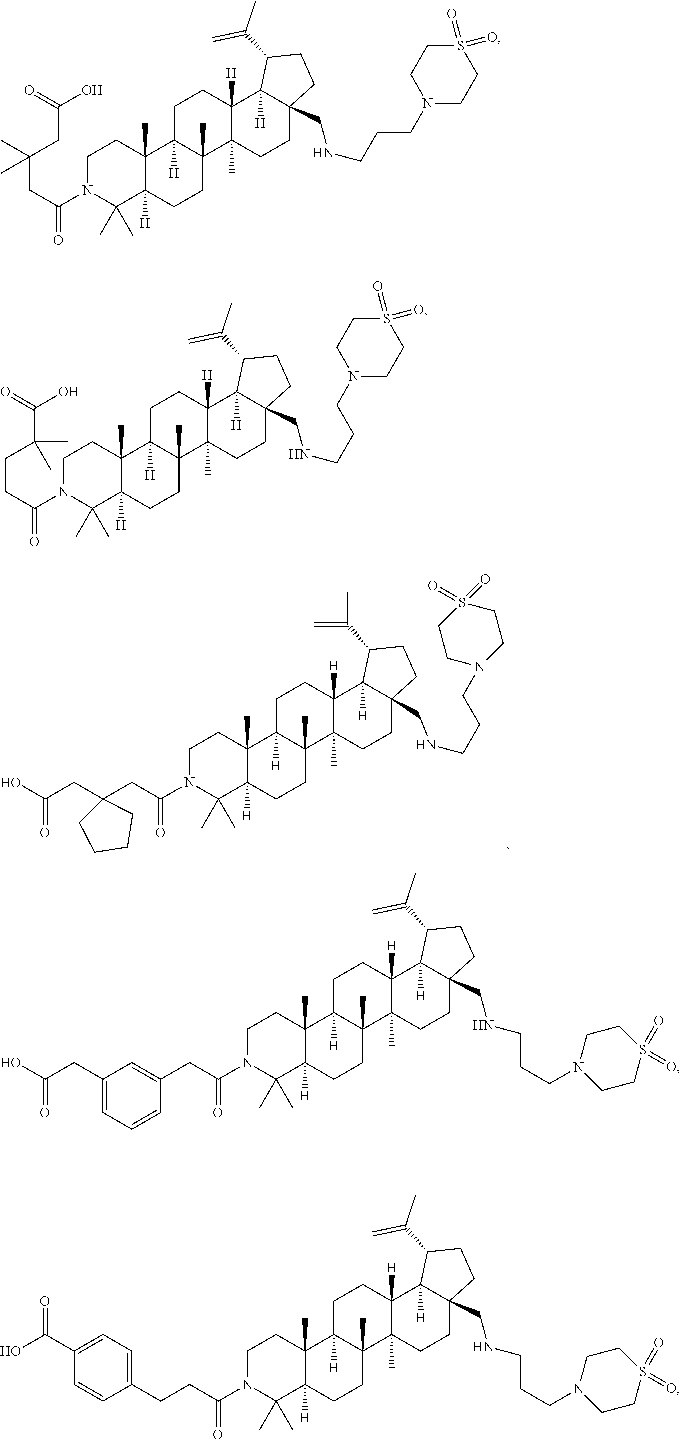

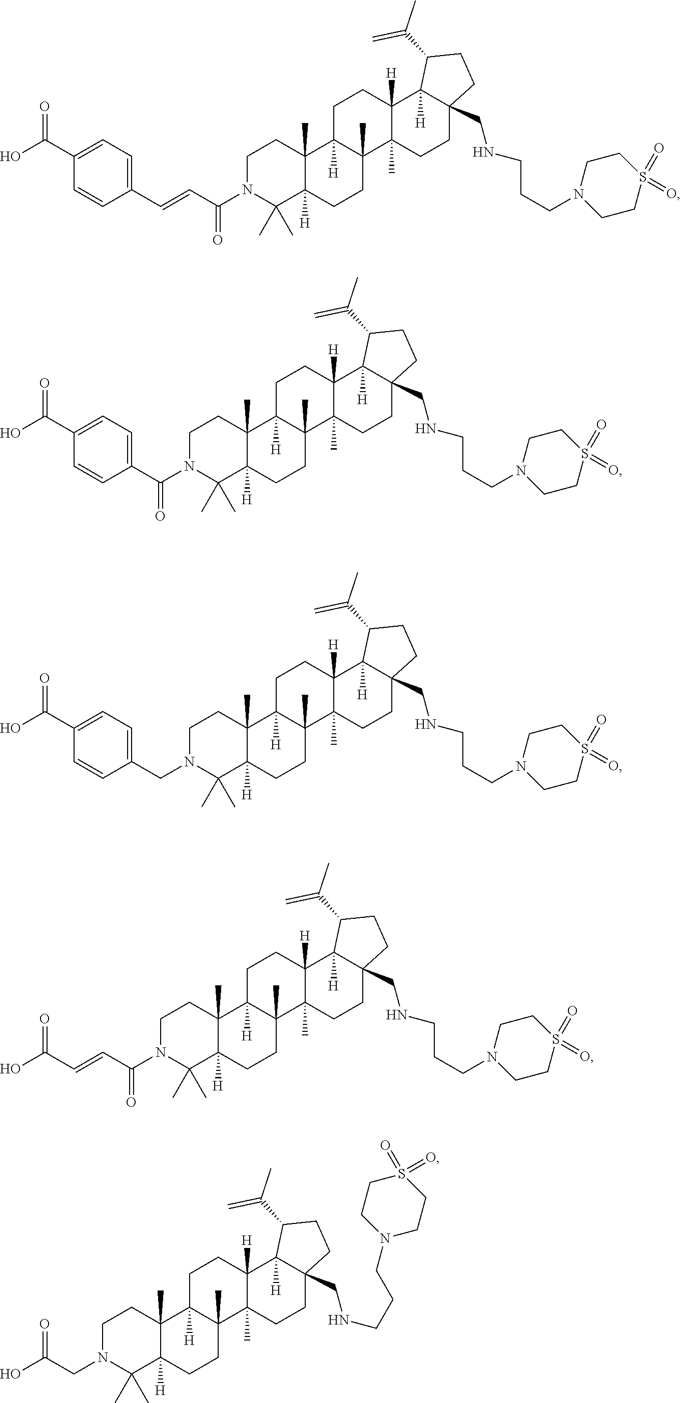

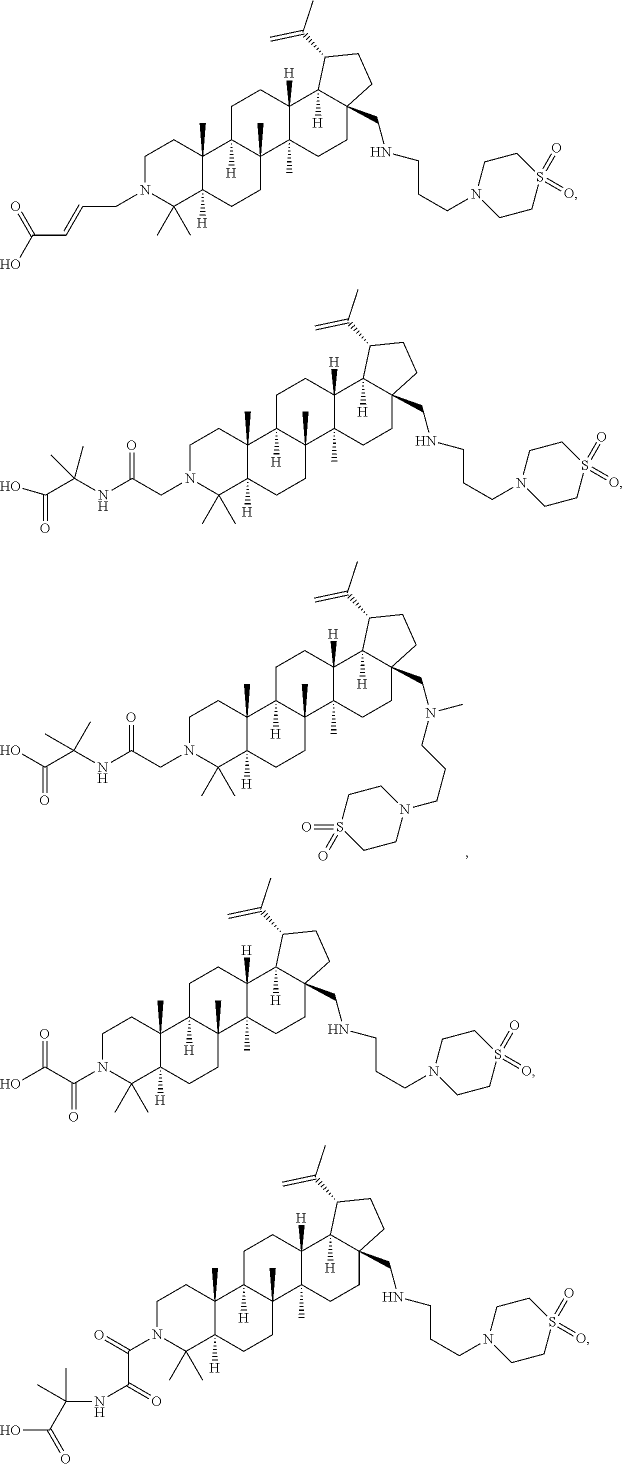

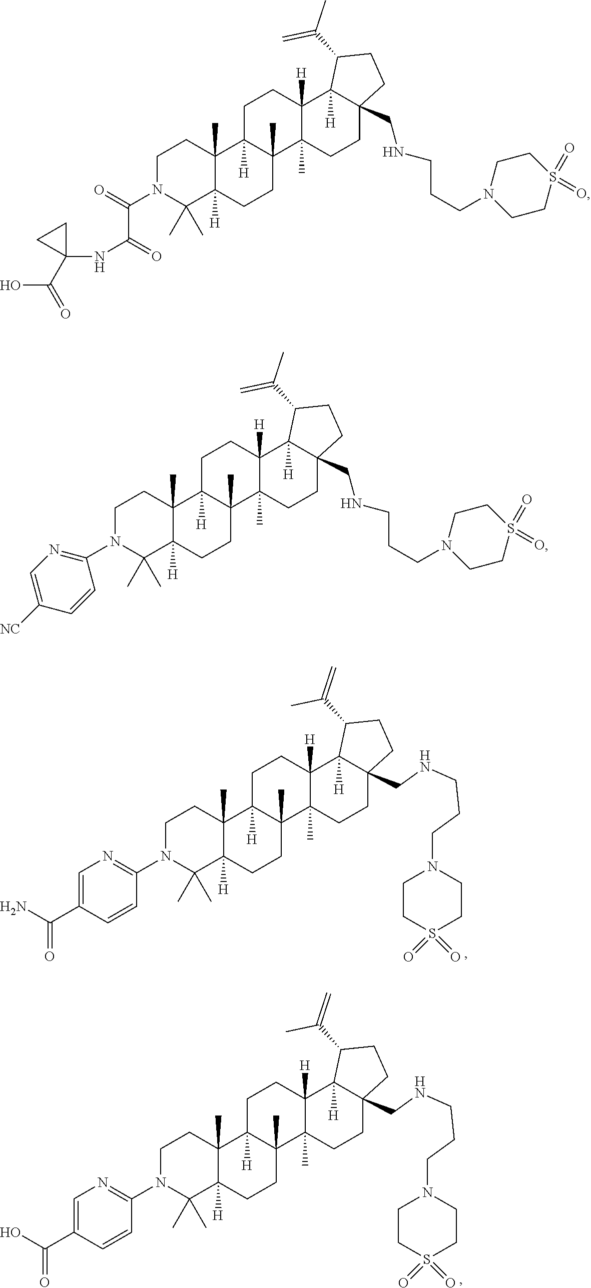

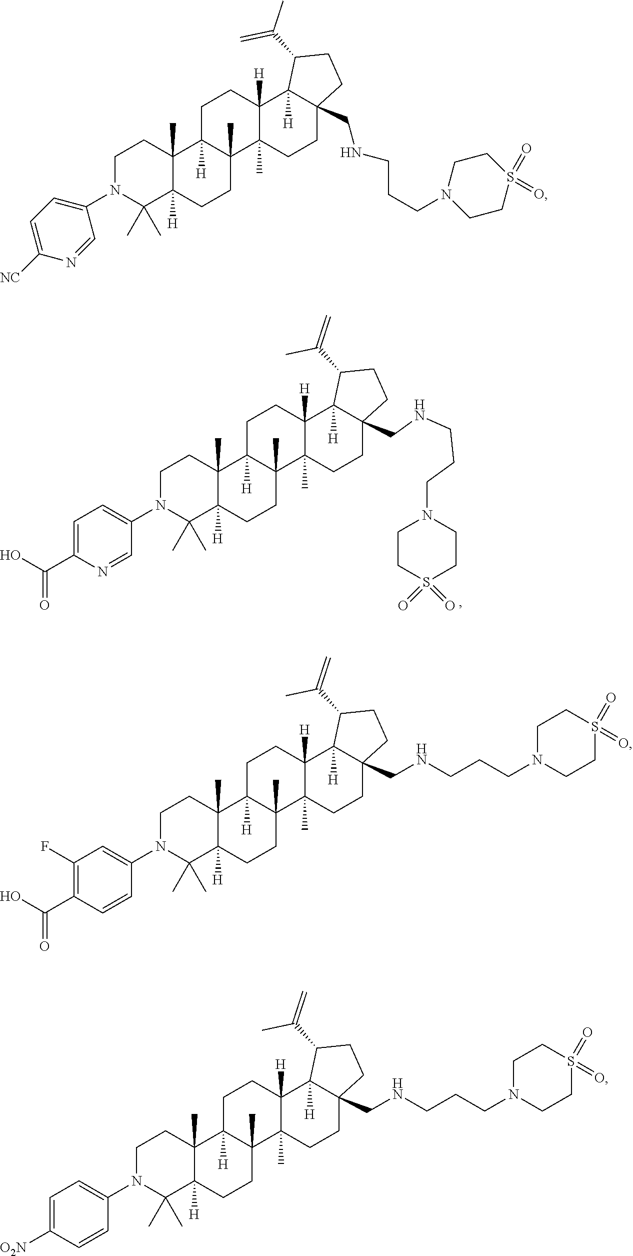

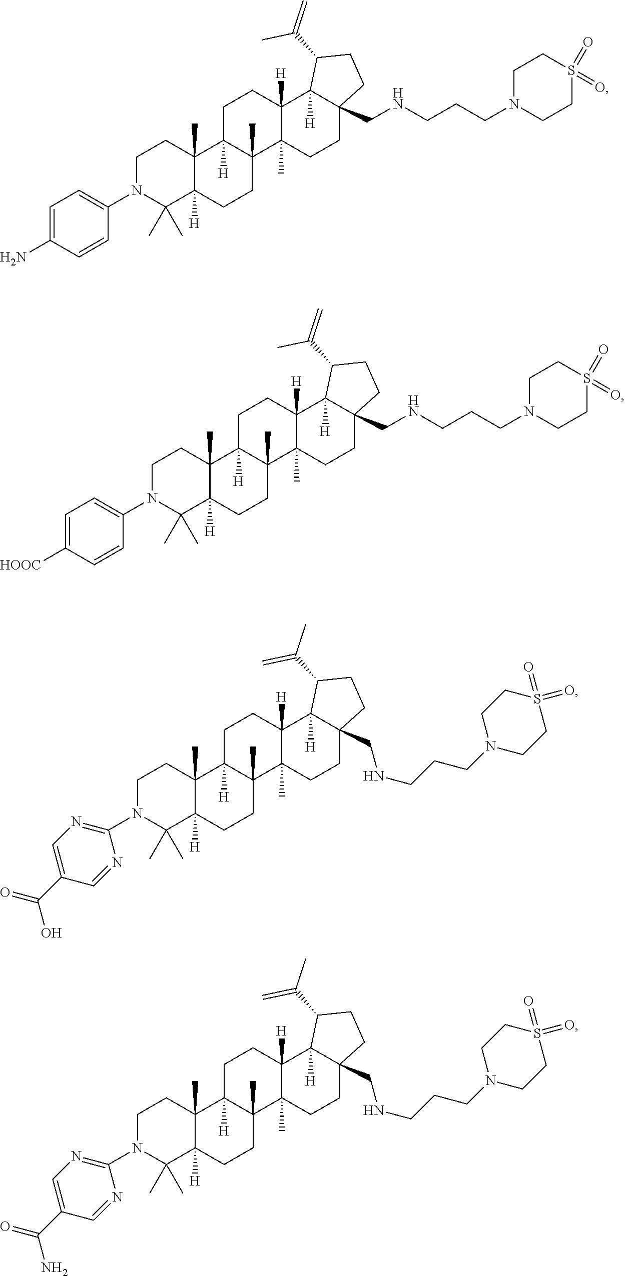

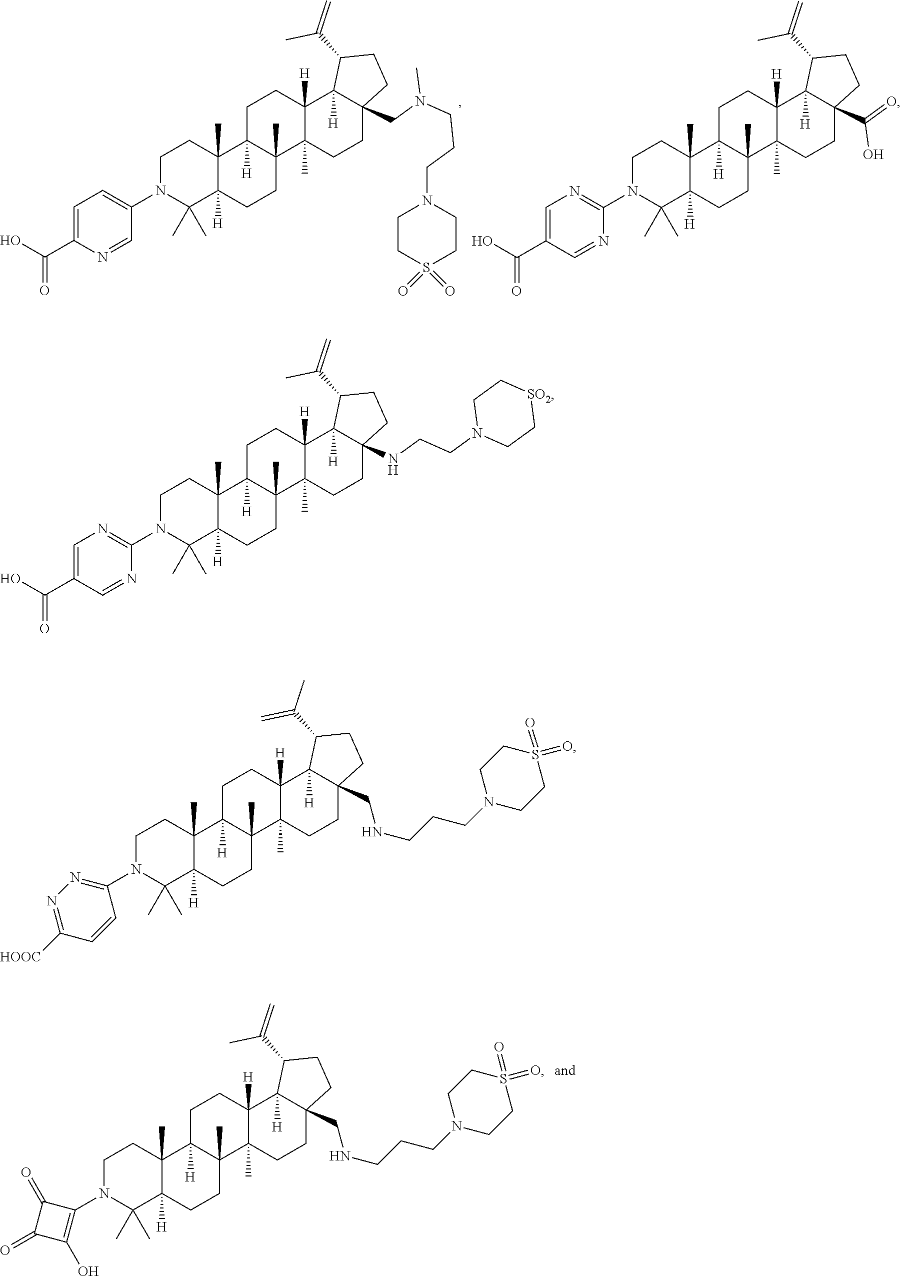

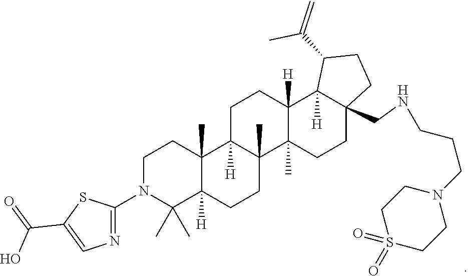

In an aspect of the invention, there is provided a composition useful for treating HIV infection comprising a therapeutic amount of a compound of Formula I, II or III and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, II or III or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I, II or III.

### Pharmaceutical Compositions and Methods of Use

The compounds of the invention herein described and set forth are generally given as pharmaceutical compositions. These compositions are comprised of a therapeutically effective amount of a compound of Formula I or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 mg of the active ingredient per dose are preferred. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 mg/mL. Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 mg/kg body weight daily. Generally, more compound is required orally and less parenterally. The specific dosing regimen, however, will be determined by a physician using sound medical judgment.

The compounds of this invention desirably have activity against HIV. Accordingly, another aspect of the invention is a method for treating HIV infection in a human patient comprising administering a therapeutically effective amount of a compound of Formula I, including a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, immunomodulators, and anti-infectives. In these combination methods, the compound of Formula I will generally be given in a daily dose of about 1-100 mg/kg body weight daily in conjunction with other agents. The other agents generally will be given in the amounts used therapeutically. The specific dosing regimen, however, will be determined by a physician using sound medical judgment.

“Combination,” “coadministration,” “concurrent” and similar terms referring to the administration of a compound of Formula I with at least one anti-HIV agent mean that the components are part of a combination antiretroviral therapy or HAART as understood by practitioners in the field of AIDS and HIV infection.

Thus, as set forth above, contemplated herein are combinations of the compounds of Formula I, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

ANTIVIRALS

| Drug Name | Manufacturer | Indication |
|---|---|---|
| Rilpivirine | Tibotec | HIV infection, AIDS, ARC |
| | | (non-nucleoside |
| | | reverse transcriptase |
| | | inhibitor) |
| COMPLERA ® | Gilead | HIV infection, AIDS, |
| | | ARC; combination |
| | | with emtricitabine, rilpivirine, |
| | | and tenofovir disoproxil |
| | | fumarate |
| 097 | Hoechst/Bayer | HIV infection, |
| | | AIDS, ARC |
| | | (non-nucleoside |
| | | reverse trans- |
| | | criptase (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® | Gilead | HIV infection, |
| (Emtricitabine) (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| SELZENTRY ™ | Pfizer | HIV infection |
| Maraviroc; (UK 427857) | | AIDs, (CCR5 antagonist, in |
| | | development) |
| TRIZIVIR ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| | | AIDs |
| | | in development |
| CMX-157 | Chimerix | HIV infection |
| Lipid conjugate of | | AIDs |
| nucleotide tenofovir | | |
| G5K1349572 | GSK | HIV infection |
| Integrase inhibitor | | AIDs |
| TIVICAY ® | | |
| dolutegravir | | |

IMMUNOMODULATORS

| Drug Name | Manufacturer | Indication |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246, 738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK & F 106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| Drug Name | Manufacturer | Indication |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

“Therapeutically effective” means the amount of agent required to provide a meaningful patient benefit as understood by practitioners in the field of AIDS and HIV infection. In general, the goals of therapeutically effective treatment include suppression of viral load, restoration and preservation of immunologic function, improved quality of life, and reduction of HIV-related morbidity and mortality.

“Patient” means a person infected with the HIV virus and suitable for therapy as understood by practitioners in the field of AIDS and HIV infection.

“Treatment,” “therapy,” “regimen,” “HIV infection,” “ARC,” “AIDS” and related terms are used as understood by practitioners in the field of AIDS and HIV infection.

### Methods of Synthesis

The compounds of the invention according to the various aspects can be made by various methods available in the art, including those of the following schemes in the specific examples which follow. The structure numbering and variable numbering shown in the synthetic schemes may be distinct from, and should not be confused with, the structure or variable numbering in the claims or the rest of the specification. The variables in the schemes are meant only to illustrate how to make some of the compounds of the invention.

Abbreviations used in the schemes generally follow conventions used in the art. Some specific chemical abbreviations used in the examples are defined as follows: “DMF” for N,N-dimethylformamide; “MeOH” for methanol; “Ar” for aryl; “TFA” for trifluoroacetic acid; “BOC” for t-butoxycarbonate, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HATU” for (1-[Bis(dimethylamino)methylene]-1H-1,2,3-triazolo[4,5-b]pyridinium 3-oxid hexafluorophosphate) “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “4” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat'd ” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tic” for thin layer chromatography, “NMR” for nuclear magnetic resonance spectroscopy, “¹H” for proton, “8” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

### EXAMPLES

The following examples illustrate typical syntheses of the compounds of Formulas I, II, and III, as described generally above. These examples are illustrative only and are not intended to limit the disclosure in any way. The reagents and starting materials are readily available to one of ordinary skill in the art.

### Chemistry

### Typical Procedures and Characterization of Selected Examples:

Unless otherwise stated, solvents and reagents were used directly as obtained from commercial sources, and reactions were performed under a nitrogen atmosphere. Flash chromatography was conducted on Silica gel 60 (0.040-0.063 particle size; EM Science supply). ¹H NMR spectra were recorded on Bruker DRX-500f at 500 MHz (or Bruker AV 400 MHz, Bruker DPX-300B or Varian Gemini 300 at 300 MHz as stated). The chemical shifts were reported in ppm on the δ scale relative to δTMS=0. The following internal references were used for the residual protons in the following solvents: CDCl₃ (δ_{H} 7.26), CD₃OD (δ_{H} 3.30), Acetic-d4 (Acetic Acid d₄) (δ_{H} 11.6, 2.07), DMSO mix or DMSO-D6_CDCl₃ ((_{H} 2.50 and 8.25) (ratio 75%:25%), and DMSO-D6 (δ_{H} 2.50). Standard acronyms were employed to describe the multiplicity patterns: s (singlet), br. s (broad singlet), d (doublet), t (triplet), q (quartet), m (multiplet), b (broad), app (apparent). The coupling constant (J) is in Hertz. All Liquid Chromatography (LC) data were recorded on a Shimadzu LC-10AS liquid chromatograph using a SPD-10AV UV-Vis detector with Mass Spectrometry (MS) data determined using a Micromass Platform for LC in electrospray mode.

### SECTION 1—EXAMPLES

### LC/MS Methods

### Method 1A

Start % B=0, Final % B=100 over 2 minute gradient, hold at 100% B Flow Rate=5 mL/min
Wavelength=220 nm
Solvent A=95% water, 5% acetonitrile, 10 mM ammonium acetate
Solvent B=5% water, 95% acetonitrile, 10 mM ammonium acetate
Column=Phenomenex Luna C18, 10 μm, 3.0×50 mm S10

### Method 2A

Start % B=0, Final % B=100 over 2 minute gradient, hold at 100% B
Flow Rate=4.0 mL/min
Wavelength=220 nm
Solvent A=90% water, 10% acetonitrile, 0.1% TFA
Solvent B=10% water, 90% acetonitrile, 0.1% TFA
Column=Sunfire C18, 5 μm, 4.6×50 mm

### Method 3A

Start % B=0, Final % B=100 over 2 minute gradient, hold at 100% B
Flow Rate=4 mL/min
Wavelength=220 nm
Solvent A=95% water, 5% methanol, 10 mM ammonium acetate
Solvent B=5% water, 95% methanol, 10 mM ammonium acetate
Column=Phenomenex Luna C18, 10 μm, 3.0×50 mm S10

### Method 4A

Start % B=30, Final % B=95 over 10 minute gradient, hold at 95% B
Flow Rate=1.2 mL/min
Detection=ELS detection
Solvent A=water, 0.1% TFA
Solvent B=acetonitrile, 0.1% TFA
Column=Cosmosil PYE 4.6×50 mm

### Method 5A

Start % B=30, Final % B=95 over 6 minute gradient, hold at 95% B
Flow Rate=1.5 mL/min
Detection=ELS detection
Solvent A=water, 10 mM ammonium acetate
Solvent B=acetonitrile, 10 mM ammonium acetate
Column=Supelco Ascentis 75×50 mm 5 μM C18

### Method 6A

Start % B=10, Final % B=95 over 12 minute gradient, hold at 95% B
Flow Rate=1 mL/min
Wavelength=220 nm
Solvent A=water with 10 mM ammonium acetate
Solvent B=acetonitrile with 10 mM ammonium acetate
Column=Waters Xbridge 4.6×150 mm 5 μM C18

### Prep HPLC Purification Methods

### Method 1

Start % B=10, Final % B=95 over 22.5 minute gradient, hold at 95% B
Flow Rate=20.0 mL/min
Detection=ELS detection
Solvent A=water, 10 mM ammonium hydroxide
Solvent B=acetonitrile, 10 mM ammonium hydroxide
Column=Waters Xbridge 21.2×150 mm 5 μM C18

### Method 2

Start % B=10, Final % B=95 over 22.5 minute gradient, hold at 95% B
Flow Rate=20.0 mL/min
Detection=ELS detection
Solvent A=water, 0.1% TFA
Solvent B=acetonitrile, 0.1% TFA
Column=Cosmosil PYE 21.2×150 mm

### Method 3

Start % B=10, Final % B=95 over 4 minute gradient, hold at 95% B
Flow Rate=20.0 mL/min
Detection=ELS detection
Solvent A=water, 10 mM Ammonium acetate
Solvent B=acetonitrile, 10 mM Ammonium acetate
Column=Waters Xbridge 21.2×150 mm 5 μM C18

### Method 4

Start % B=10, Final % B=95 over 22.5 minute gradient, hold at 95% B
Flow Rate=20.0 mL/min
Detection=ELS detection
Solvent A=water, 0.1% Formic acid
Solvent B=acetonitrile, 0.1% Formic acid
Column=Cosmosil PYE 21.2×150 mm

### Preparation of Key Intermediates

### Preparation of ((7aR,7bR,9aS,12R,12aR,12bR,14aR,14bR)-5,5,7a,7b,14b-pentamethyl-12-(prop-1-en-2-yl)docosahydrocyclopenta[7,8]phenanthro[2,1-c]azepin-9a-yl)methanol

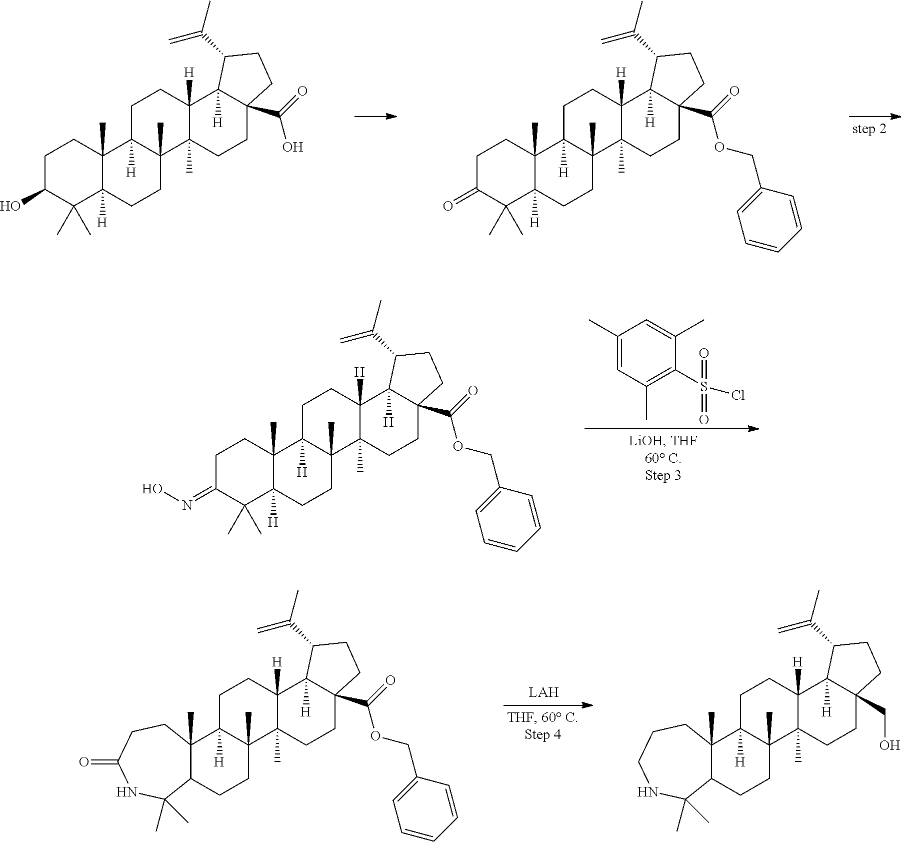

Step 1. Preparation of (1R,3aS,5aR,5bR,7aR,11aR,11bR,13aR,13bR,E)-benzyl 9-(hydroxyimino)-5a,5b,8,8,11a-pentamethyl-1-(prop-1-en-2-yl)icosahydro-1H-cyclopenta[a]chrysene-3a-carboxylate.

To a solution of (1R,3aS,5aR,5bR,7aR,11aR,11bR,13aR,13bR)-benzyl 5a,5b,8,8,11a-pentamethyl-9-oxo-1-(prop-1-en-2-yl)icosahdro-1H-cyclopenta[a]chrysene-3a-carboxylate (3.0 g, 5.51 mmol) prepared as described in WO2011/153315 and potassium hydroxide (1.545 g, 27.5 mmol) in methanol (50 mL) was added hydroxylamine hydrochloride (0.765 g, 11.01 mmol) followed by toluene (30 mL). The mixture was heated to reflux for 15 h, then was cooled to rt. The solvent was removed under reduced pressure and the residue was diluted with water then extracted with diethyl ether three times. The organic layers were dried over magnesium sulfate, filtered and concentrated under reduced pressure to give the title product (3.03 g, 5.41 mmol, 98% yield) as a white solid. ¹H NMR (500 MHz, chloroform-d) δ 7.79 (br. s., 1H), 7.39-7.28 (m, 5H), 5.15 (d, J=12.2 Hz, 1H), 5.08 (d, J=12.5 Hz, 1H), 4.72 (d, J=2.1 Hz, 1H), 4.59 (dd, J=2.1, 1.2 Hz, 1H), 3.01 (td, J=11.0, 4.6 Hz, 1H), 2.97-2.90 (m, 1H), 2.30-2.15 (m, 3H), 1.94-1.81 (m, 2H), 1.80-1.72 (m, 1H), 1.67 (s, 3H), 1.11 (s, 3H), 1.02 (s, 3H), 0.92 (s, 3H), 0.89 (s, 3H), 0.78 (s, 3H), 1.72-0.73 (m, 17H).

Step 2. Preparation of (7aR,7bR,9aS,12R,12aR,12bR,14aR,14bR)-benzyl 5,5,7a,7b,14b-pentamethyl-3-oxo-12-(prop-1-en-2-yl)docosahydrocyclopenta[7,8]phenanthro[2,1-c]azepine-9a-carboxylate.

To a solution of (1R,3aS,5aR,5bR,7aR,11aR,11bR,13aR,13bR,E)-benzyl 9-(hydroxyimino)-5a,5b,8,8,11a-pentamethyl-1-(prop-1-en-2-yl)icosahydro-1H-cyclopenta[a]chrysene-3a-carboxylate (3.0 g, 5.36 mmol) in THF (50 mL) was added lithium hydroxide (0.257 g, 10.72 mmol). The mixture was stirred at rt for 20 minutes then 2-mesitylenesulfonyl chloride (5.86 g, 26.8 mmol) was added portionwise over 30 minutes. The mixture was stirred for 1 h at rt then was heated to 60° C. for 22 h. The mixture was cooled to rt, diluted with water (150 mL) and extracted with ether (3×150 mL). The combined organic layers were dried over sodium sulfate, filtered and concentrated under reduced pressure. The residue was purified by flash chromatography using a 90 g silica gel column and a 0-75% ethyl acetate in hexanes gradient. The title product (1.78 g, 3.02 mmol, 56.4% yield) was isolated as a white foam. LCMS: m/e 560.5 (M+H)⁺, 2.13 min (method 1A).

Step 3. Preparation of (5aR,7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-3(2H)-one.

To a solution of benzyl (5aR,7aR,7bR,9aS,12R,12aR,12bR,14aR,14bR)-5,5,7a,7b,14b-pentamethyl-3-oxo-12-(prop-1-en-2-yl)icosahydrocyclopenta[7,8]phenanthro[2,1-c]azepine-9a(1H)-carboxylate (0.25 g, 0.447 mmol) in THF (2 mL) was added LAH (1M in THF) (0.893 mL, 0.893 mmol) dropwise. The mixture was stirred at rt for 3.5 h. The mixture was carefully quenched with water, neutralized with saturated aqueous ammonium chloride and extracted with ether (3×10 mL). The combined organic layer were dried over sodium sulfate, filtered and concentrated under reduced pressure. The residue was purified by flash chromatography using a 0-10% methanol in dichloromethane gradient. The title compound was isolated as a white solid (0.095 g, 0.208 mmol, 47% yield). LCMS: m/e 456.44 (M+H)⁺, 2.03 min (method 2A).

Step 4. To a solution of (7aR,7bR,9aS,12R,12aR,12bR,14aR,14bR)-benzyl 5,5,7a,7b,14b-pentamethyl-3-oxo-12-(prop-1-en-2-yl)docosahydrocyclopenta[7,8]phenanthro[2,1-c]azepine-9a-carboxylate (0.25 g, 0.447 mmol) in THF (2 mL) was added LAH (1M solution in THF) (1 mL, 1.000 mmol) dropwise. The mixture was heated to 60° C. After 17 h of heating, the mixture was cooled to rt. An additional 0.5 mL of 1M LAH solution was added and the mixture was heated to 60° C. for 6 h. The reaction was quenched by carefully adding water. The mixture was extracted with ether (2×25 mL). The combined organic layers were dried over sodium sulfate, filtered and concentrated under reduced pressure. The crude product was used in the next step with no additional purification. LCMS: m/e 442.4 (M+H)⁺, 1.36 min (method 2A).

### Preparation of benzyl(1R,3aS,5aR,5bR,7aR,10aR,10bR,12aR,12bR)-5a,5b,8,8,10a-pentamethyl-9-oxo-1-(prop-1-en-2-yl)octadecahydrodicyclopenta[a,i]phenanthrene-3a(1H)-carboxylate

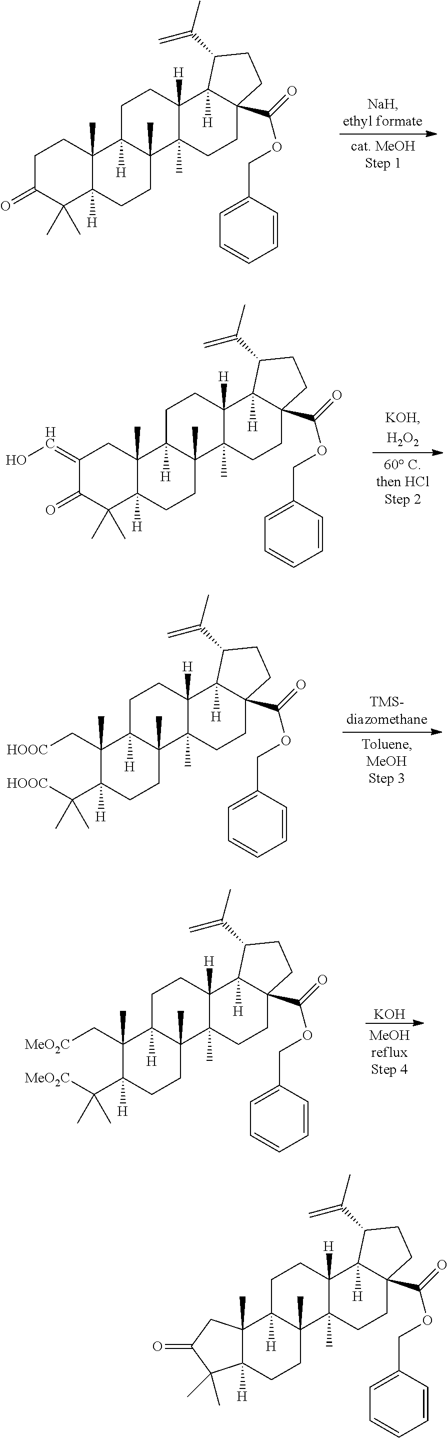

Step 1: Preparation of benzyl(1R,3aS,5aR,5bR,7aR,11aR,11bR,13aR,13bR,Z)-10-(hydroxymethylene)-5a,5b,8,8,11a-pentamethyl-9-oxo-1-(prop-1-en-2-yl)icosahydro-3aH-cyclopenta[a]chrysene-3a-carboxylate.

To a solution of benzyl (1R,3aS,5aR,5bR,7aR,11aR,11bR,13aR,13bR)-5a,5b,8,8,11a-pentamethyl-9-oxo-1-(prop-1-en-2-yl)icosahydro-3aH-cyclopenta[a]chrysene-3a-carboxylate (10.87 g, 19.95 mmol) in toluene (150 mL) was added pyridine (15.0 mL) followed by sodium hydride (7.0 g, 175 mmol) (portionwise over 15 minutes). The mixture was stirred for five minutes and ethyl formate (32.5 mL, 399 mmol) was added dropwise followed by 5 drops of methanol. The mixture was stirred at rt for 6 h, then was carefully quenched with 0.1N HCl (100 mL). The mixture was diluted further with water (100 mL) and extracted with diethyl ether (3×150 mL). The combined organic layers were dried over sodium sulfate, filtered and concentrated under reduced pressure to give the title product (11.43 g, 19.95 mmol, 100% yield), as a thick, yellow oil. ¹H NMR (400 MHz, chloroform-d) δ 14.86 (d, J=3.3 Hz, 1H), 8.58 (d, J=3.0 Hz, 1H), 7.42-7.29 (m, 5H), 5.20-5.08 (m, 2H), 4.75 (d, J=2.3 Hz, 1H), 4.61 (dd, J=2.3, 1.3 Hz, 1H), 3.04 (td, J=10.9, 4.9 Hz, 1H), 2.38-2.20 (m, 3H), 1.70 (s, 3H), 1.18 (s, 3H), 1.10 (s, 3H), 0.97 (s, 3H), 0.82 (s, 6H), 2.01-0.74 (m, 19H).

Step 2: Preparation of 2-((3R,4R,5R,8R,9R,10R,13S,14R,15R)-13-((benzyloxy)carbonyl)-4-(carboxymethyl)-4,9,10-trimethyl-15-(prop-1-en-2-yl)hexadecahydro-1H-cyclopenta[a]phenanthren-3-yl)-2-methylpropanoic acid. A suspension of benzyl (1R,3aS,5aR,5bR,7aR,11aR,11bR,13aR,13bR,Z)-10-(hydroxymethylene)-5a,5b,8,8,11a-pentamethyl-9-oxo-1-(prop-1-en-2-yl)icosahdro-3aH-cyclopenta[a]chrysene-3a-carboxylate (11.43 g, 19.95 mmol) in potassium hydroxide (2 M solution) (150 mL, 299 mmol) and methanol (50 mL) was heated to 60° C. To the suspension was added hydrogen peroxide (30%) (61.1 mL, 599 mmol) dropwise. The mixture was stirred for 3 h at 60° C., then was cooled to rt, acidified with 1N HCl to pH=1 and extracted with ether (3×150 mL). The organic layer was dried over sodium sulfate, filtered, and concentrated under reduced pressure. The crude product was used in the next step with no additional purification. LC/MS: m/e 591.47 (M−H)⁻, 1.43 min (method 1A).

Step 3: Preparation of benzyl(3R,4R,5R,8R,9R,10R,13S,14R,15R)-3-(1-methoxy-2-methyl-1-oxopropan-2-yl)-4-(2-methoxy-2-oxoethyl)-4,9,10-trimethyl-15-(prop-1-en-2-yl)hexadecahydro-13H-cyclopenta[a]phenanthrene-13-carboxylate.

The crude residue from the previous step was dissolved in Toluene (100 mL) and methanol (25 mL) and was cooled to 0° C. To the solution was added TMS-Diazomethane (23.94 mL, 47.9 mmol). The mixture was warmed to rt. After stirring the mixture for 3 h at rt, it was quenched with sat. aq. sodium bicarbonate (300 mL) and was extracted with diethyl ether (3×150 mL). The combined organic layers were dried over sodium sulfate, filtered and concentrated under reduced pressure. The crude product was used directly in the next step with no additional purification. LC/MS: m/e 621.48 (M+H)⁺, 2.28 min (method 1A).

Step 4: To a mixture of the product from step 3 (12.39 g, 19.95 mmol) in methanol (200 mL) was added potassium hydroxide (22.39 g, 399 mmol). The mixture was heated to reflux for 16 h then was cooled to rt. The solvent was removed under reduced pressure, the residue was diluted with water (300 mL) and was extracted with ether (3×150 mL). The combined organic layers were dried over magnesium sulfate, filtered and concentrated under reduced pressure. Benzyl (1R,3aS,5aR,5bR,7aR,10aR,10bR,12aR,12bR)-5a,5b,8,8,10a-pentamethyl-9-oxo-1-(prop-1-en-2-yl)octadecahydrodicyclopenta[a,i]phenanthrene-3a(1H)-carboxylate was isolated as a thick yellow oil which was used in the next step with no additional purification. ¹H NMR (500 MHz, chloroform-d) δ 7.42-7.29 (m, 5H), 5.19-5.08 (m, 2H), 4.73 (d, J=2.1 Hz, 1H), 4.60 (dd, J=2.3, 1.4 Hz, 1H), 3.02 (td, J=11.0, 4.9 Hz, 1H), 1.68 (s, 3H), 1.01 (s, 3H), 1.00 (s, 3H), 0.96 (s, 3H), 0.82 (d, J=0.9 Hz, 3H), 0.78 (s, 3H), 2.40-0.67 (m, 22H).

### Preparation of ((4aR,6aR,6bR,8aS,11R,11aR,11bR,13aR,13bR)-4,4,6a,6b,13b-pentamethyl-11-(prop-1-en-2-yl)icosahdro-8aH-cyclopenta[5,6]naphtho[2,1-f]isoquinolin-8a-yl)methanol

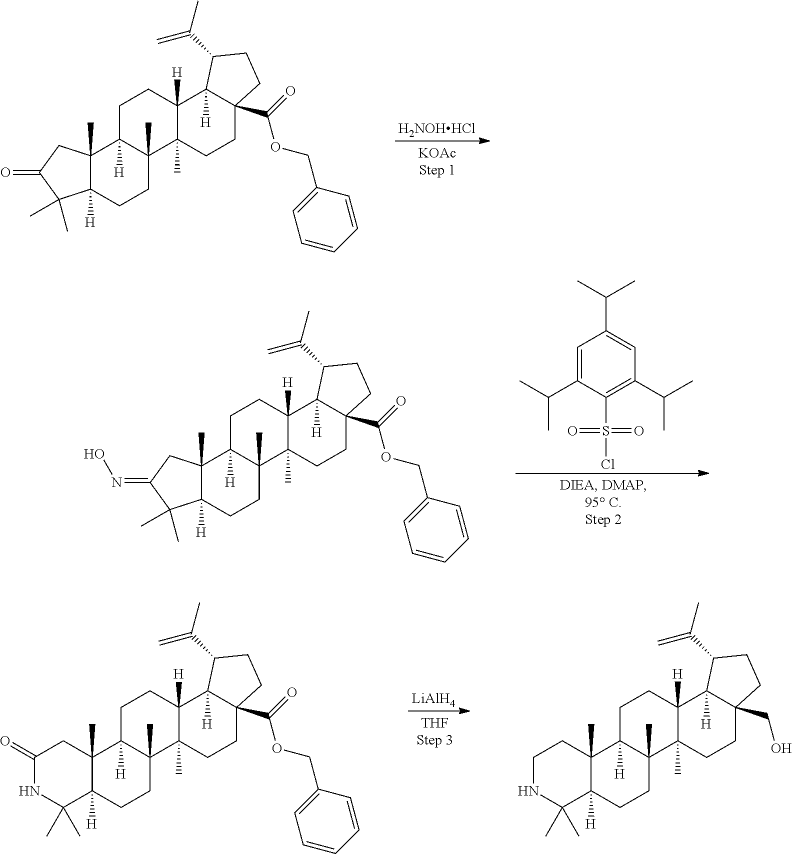

Step 1: Preparation of benzyl(1R,3aS,5aR,5bR,7aR,10aR,10bR,12aR,12bR,E)-9-(hydroxyimino)-5a,5b,8,8,10a-pentamethyl-1-(prop-1-en-2-yl)octadecahydrodicyclopenta[a]phenanthrene-3a(1H)-carboxylate.

To a solution of benzyl (1R,3aS,5aR,5bR,7aR,10aR,10bR,12aR,12bR)-5a,5b,8,8,10a-pentamethyl-9-oxo-1-(prop-1-en-2-yl)octadecahydrodicyclopenta[a,i]phenanthrene-3a(1H)-carboxylate (5.3 g, 9.99 mmol) in toluene (100 mL) was added potassium hydroxide (2.80 g, 49.9 mmol) followed by hydroxylamine HCl (1.388 g, 19.97 mmol). The mixture was heated to reflux with a dean-stark trap containing 4A molecular sieves attached. After 3 h of heating, the mixture was cooled to rt. Starting material still remained while a new peak was visible by TLC. To the mixture was added 1.6 g of hydroxylamine HCl and 20 mL of MeOH. The mixture was attached to the Dean-Stark trap and was heated to reflux. After 19 h of heating, the mixture was cooled to rt. TLC indicated starting material still remained. The Dean-Stark trap was empties of solvent and the molecular sieves were replaced with dry sieves. To the mixture was added 20 mL of MeOH and and 1.4 g of hydroxylamine HCl. The mixture was heated to reflux overnight. TLC indicated starting material still remained. The Dean-Stark trap was empties of solvent and the molecular sieves were replaced with dry sieves. To the mixture was added 20 ML of MeOH and and 1.4 g of hydroxylamine HCl. The mixture was again heated to reflux overnight. TLC indicated starting material still remained. To the mixture was added 20 ML of MeOH and and 1.4 g of hydroxylamine HCl. The mixture was heated to reflux for 6 h, then was cooled to rt. TLC showed no remaining starting material. Solids were removed from the mixture by filtration. The filtrate was partitioned with water (150 mL) and extracted with ethyl acetate (3×125 mL). The combined organic layers were dried over sodium sulfate, filtered and concentrated under reduced pressure. The residue was purified by flash chromatography using a 0-20% ethyl acetate in hexanes gradient. The fractions containing the product were combined and concentrated under reduced pressure to give the title product (4.53 g, 8.30 mmol, 83% yield) as an off-white foam. LC/MS: m/e 546.44 (M+H)⁺, 2.37 min (method 1A).

Step 2: Preparation of benzyl(4aR,6aR,6bR,8aS,11R,11aR,11bR,13aR,13bR)-4,4,6a,6b,13b-pentamethyl-2-oxo-11-(prop-1-en-2-yl)icosahydro-8aH-cyclopenta[5,6]naphtho[2,1-f]isoquinoline-8a-carboxylate.

To a solution of benzyl (1R,3aS,5aR,5bR,7aR,10aR,10bR,12aR,12bR,E)-9-(hydroxyimino)-5a,5b,8,8,10a-pentamethyl-1-(prop-1-en-2-yl)octadecahydrodicyclopenta[a,i]phenanthrene-3a(1H)-carboxylate (5.2 g, 9.53 mmol) in toluene (125 mL) was added 2,4,6-triisopropylbenzene-1-sulfonyl chloride (8.3 g, 27.4 mmol), followed by DIEA (3.4 mL, 19.52 mmol) and a catalytic amount of 4-DMAP (25 mg). The mixture was stirred for 2 hours at room temperature before it was heated in an oil bath at 95° C. After heating the mixture overnight, it was diluted with 50 mL 0.5N HCl. The organic phase was separated and washed with water. The aq. HCl phase was extracted with ethyl acetate (2×25 mL). The organic layers were combined, concentrated and purified on a 90 gm silica gel column eluted with mixtures of ethyl acetate and hexanes to give 4.1 gm (79%) of the titled compound as a white solid. LC/MS: m/e 546.49 (M+H)⁺, 3.78 min (method 2A); ¹H NMR (500 MHz, CDCl₃) δ 7.41-7.30 (m, 5H), 5.62 (s, 1H), 5.21-5.07 (m, 2H), 4.75 (s, 1H), 4.63 (s, 1H), 3.03 (td, J=10.9, 4.4 Hz, 1H), 2.43 (d, J=16.8 Hz, 1H), 2.31 (d, J=12.8 Hz, 1H), 2.27-2.18 (m, 1H), 1.98-1.84 (m, 2H), 1.80-1.71 (m, 2H), 1.70 (s, 3H), 1.62 (t, J=11.4 Hz, 1H), 1.50-1.37 (m, 9H), 1.37-1.26 (m, 4H), 1.25 (s, 3H), 1.15 (s, 3H), 1.10-1.01 (m, 1H), 0.98 (s, 3H), 0.95 (s, 3H), 0.79 (s, 3H); ¹³C NMR (126 MHz, CDCl₃) δ 175.8, 170.8, 150.4, 136.5, 128.6, 128.4, 128.2, 109.9, 65.9, 56.6, 55.4, 51.6, 49.4, 48.5, 47.4, 47.0, 42.5, 40.6, 38.2, 37.0, 36.9, 33.9, 33.1, 32.1, 30.7, 29.7, 25.4, 21.6, 21.2, 20.2, 19.5, 16.8, 15.4, 14.7.

Step 3: To a solution of benzyl (4aR,6aR,6bR,8aS,11R,11aR,11bR,13aR,13bR)-4,4,6a,6b,13b-pentamethyl-2-oxo-11-(prop-1-en-2-yl)icosahydro-8aH-cyclopenta[5,6]naphtho[2,1-f]isoquinoline-8a-carboxylate (5a, 4.34 gm, 7.95 mmol) in THF (90 mL) at 0° C. was added LiAlH₄ (2.0 gm, 52.7 mmol) in small portions. The mixture was warmed to room temperature for 30 minutes then was further warmed to 65° C. for 48 hours. The reaction mixture was quenched by slow addition of 20% w/w solution of sodium hydroxide until a free-flowing, white suspension was formed. The mixture was filtered and concentrated into a syrup. It was redissolved in chloroform then was passed through a short bed of silica gel which was washed with mixtures of isopropanol and methylene chloride to give ((4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methanol (2.9 gm, 85%) after concentration. LC/MS: m/e 428.24 (M+H)⁺, 1.87 min (method 3A); ¹H NMR (400 MHz, CDCl₃) δ 4.70 (d, J=2.0 Hz, 1H), 4.60 (dd, J=2.3, 1.3 Hz, 1H), 3.81 (d, J=9.5 Hz, 1H), 3.35 (d, J=11.0 Hz, 1H), 3.10-2.95 (m, 1H), 2.97-2.83 (m, 1H), 2.41 (td, J=10.8, 5.8 Hz, 1H), 2.00 (s, 3H), 1.95 (ddd, J=13.5, 7.1, 3.0 Hz, 2H), 1.87 (dd, J=12.0, 8.0 Hz, 2H), 1.68-1.61 (m, 2H), 1.61-1.53 (m, 2H), 1.49 (d, J=10.8 Hz, 2H), 1.44-1.34 (m, 6H), 1.25 (dd, J=13.3, 3.3 Hz, 2H), 1.19 (s, 3H), 1.15 (d, J=5.0 Hz, 1H), 1.11 (s, 3H), 1.09-1.08 (m, 3H), 1.05 (s, 3H), 1.00 (s, 3H), 0.93 (s, 3H); ¹³C NMR (101 MHz, CDCl₃) δ 150.1, 109.4, 60.1, 55.2, 53.6, 49.6, 48.3, 47.4, 47.4, 42.4, 40.6, 38.7, 36.9, 36.7, 35.6, 33.6, 33.4, 31.4, 29.3, 28.8, 26.6, 24.7, 24.5, 20.1, 19.1, 18.7, 15.6, 15.5, 14.4.

### General Method A for Amide Synthesis

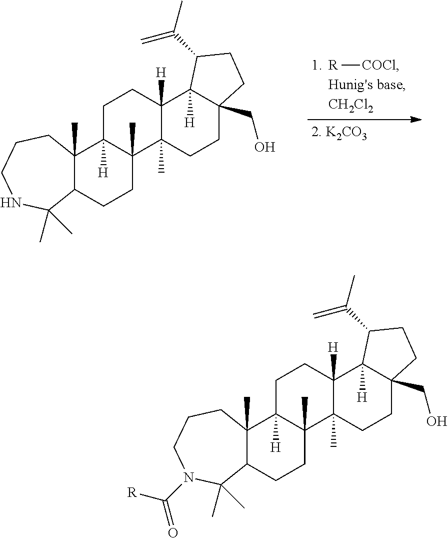

To a vial containing a solution of ((5aR,7aR,7bR,9aS,12R,12aR,12bR,14aR,14bR)-5,5,7a,7b,14b-pentamethyl-12-(prop-1-en-2-yl)icosahydrocyclopenta[7,8]phenanthro[2,1-c]azepin-9a(1H)-yl)methanol (450 mg, 1.02 mmol) in dichloromethane (20.0 mL) was added Hunig's base (1.78 mL, 10.19 mmol) to prepare a stock solution of the template. To separate vials containing the reactant acid chlorides (1.019 mmol) was added 2.0 mL of the stock solution of template. Each vial was sealed and shaken vigorously overnight at room temperature. Once analysis showed the reactions were complete, they were concentrated at 35° C. Each residue was diluted with 1.0 mL of methanol, and was sonicated until all solids dissolved. To each vial was added 0.401 mL of 2M potassium carbonate solution. The vials were heated to 60° C. overnight. Analysis at this point showed a mixture of N and 0 alkylated products that were purified by prep HPLC equipped with an ELS detector to initiate collection. Several of the compounds were not completely hydrolyzed using the above conditions and had to be further subjected to hydrolysis conditions to complete the saponification.

### Example 1

### Preparation of 3-((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)-3-oxopropanoic acid

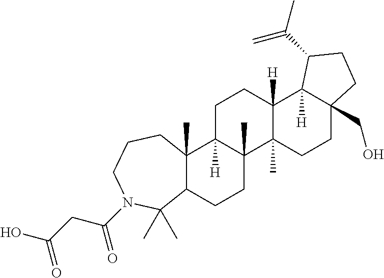

The preparation of 3-oxo-3-(((7aR,7bR,9aS,12R,12aR,12bR,14aR,14bR)-5,5,7a,7b,14b-pentamethyl-12-(prop-1-en-2-yl)icosahydrocyclopenta[7,8]phenanthro[2,1-c]azepin-9a(1H)-yl)methoxy)propanoic acid and 3-((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)-3-oxopropanoic acid was accomplished using general method A for amide synthesis. Methyl 3-chloro-3-oxopropanoate (0.139 g, 1.019 mmol) was used as the reactant acid chloride. The material was purified using prep HPLC purification method 1, then the individual products were repurified using prep HPLC purification method 2. LCMS: m/e 528.50 (M+H)⁺, 7.43 min (method 4A).

### Example 2

### Preparation of 3-((7aR,7bR,9aS,12R,12aR,12bR,14aR,14bR)-9a-(hydroxymethyl)-5,5,7a,7b,14b-pentamethyl-12-(prop-1-en-2-yl)docosahydrocyclopenta[7,8]phenanthro[2,1-c]azepine-4-carbonyl)benzoic acid

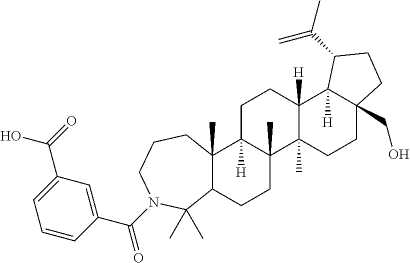

The preparation of 3-((7aR,7bR,9aS,12R,12aR,12bR,14aR,14bR)-9a-(hydroxymethyl)-5,5,7a,7b,14b-pentamethyl-12-(prop-1-en-2-yl)docosahydrocyclopenta[7,8]phenanthro[2,1-c]azepine-4-carbonyl)benzoic acid was accomplished using general method A for amide synthesis. Methyl 3-(chlorocarbonyl)benzoate (0.202 g, 1.019 mmol) was used as the reactant acid chloride. The material was purified using prep HPLC purification method 1. LCMS: m/e 590.55 (M+H)⁺, 7.79 min (method 4A).

### Example 3

### Preparation of 4-((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)-4-oxobutanoic acid

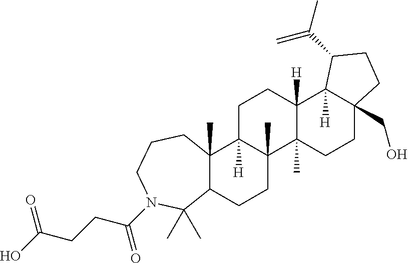

The preparation of 4-((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)-4-oxobutanoic acid was accomplished using general method A for amide synthesis. Ethyl 4-chloro-4-oxobutanoate (0.168 g, 1.019 mmol) was used as the reactant acid chloride. The material was purified using prep HPLC purification method 1. LCMS: m/e 542.50 (M+H)⁺, 7.03 min (method 4A).

### Example 4

### Preparation of 6-((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)-6-oxohexanoic acid

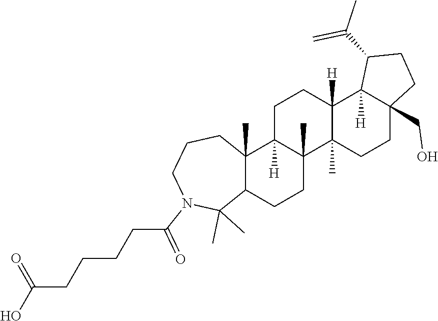

The preparation of 6-((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)-6-oxohexanoic acid was accomplished using general method A for amide synthesis. Methyl 6-chloro-6-oxohexanoate (0.168 g, 1.019 mmol) was used as the reactant acid chloride. The material was purified using prep HPLC purification method 1. LCMS: m/e 570.55 (M+H)⁺, 7.26 min (method 4A).

### General Method B for Amide Synthesis

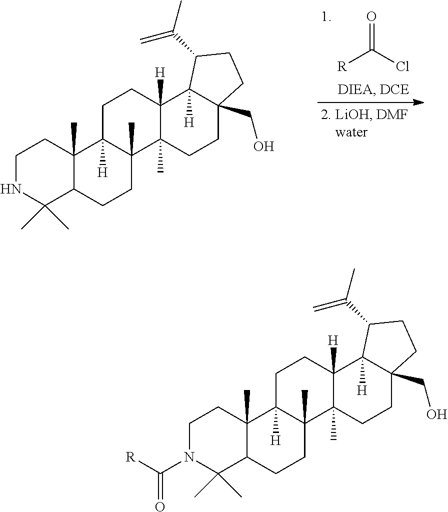

To a vial containing ((6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methanol (0.480 g, 1.122 mmol) and DCE (24 mL) was added DIEA (0.588 mL) to prepare the stock solution of template. To vials containing the reactant acid chlorides (1.0 equiv., 0.094 mmol per reaction) was added 2.0 mL of the stock solution and the mixtures were shaken vigorously overnight at rt. The reaction mixtures were concentrated then diluted with 2.0 mL of DMF and sonicated to fully dissolve the mixtures. To each vial was added lithium hydroxide monohydrate (10.2 equiv, 39.0 mg, 0.93 mmol) and water (1 mL). The mixtures were allowed to shake at rt for 2 days then were concentrated. Each mixture was diluted with methanol (1.2 mL) and DMF (0.4 mL) and was shaken vigorously. 37% HCl (0.05 mL) was added and the mixtures were sonicated until solids dissolved. Each mixture was then purified by prep HPLC.

### Example 5

### Preparation of 3-((6aR,6bR,8aS,11R,11aR,11bR,13aR,13bR)-8a-(hydroxymethyl)-4,4,6a,6b,13b-pentamethyl-11-(prop-1-en-2-yl)icosahydro-1H-cyclopenta[5,6]naphtho[2,1-f]isoquinoline-3-carbonyl)benzoic acid

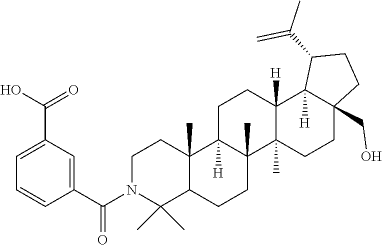

Preparation of 3-((6aR,6bR,8aS,11R,11aR,11bR,13aR,13bR)-8a-(hydroxymethyl)-4,4,6a,6b,13b-pentamethyl-11-(prop-1-en-2-yl)icosahydro-1H-cyclopenta[5,6]naphtho[2,1-f]isoquinoline-3-carbonyl)benzoic acid was accomplished using the general method B for amide synthesis. 3-(chlorocarbonyl)benzoic acid methyl ester was used as the reactant acid chloride. The material was purified using prep HPLC purification method 3. LCMS: m/e 576.34 (M+H)⁺, 9.02 min (method 6A).

### Example 6

### Preparation of 2-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-2-oxoacetic acid

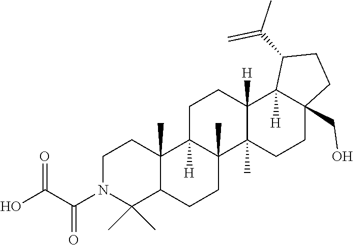

Preparation of 2-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-2-oxoacetic acid was accomplished using the general method B for amide synthesis. Ethyl 2-chloro-2-oxoacetate was used as the reactant acid chloride. The material was purified using prep HPLC purification method 3. LCMS: m/e 500.27 (M+H)⁺, 7.59 min (method 6A).

### Example 7

### Preparation of 3-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-3-oxopropanoic acid

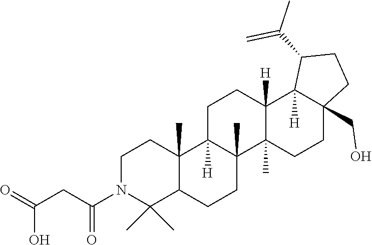

The preparation of 3-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-3-oxopropanoic acid was accomplished using the general method B for amide synthesis. Methyl 3-chloro-3-oxopropanoate was used as the reactant acid chloride. The material was purified using prep HPLC purification method 4. LCMS: m/e 514.28 (M+H)⁺, 7.92 min (method 6A).

### Example 8

### Preparation of 5-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-5-oxopentanoic acid

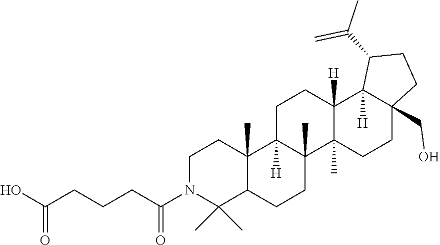

To a mixture of ((6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methanol (0.051 g, 0.119 mmol) in DCE (2 mL) was added Hunig's Base (0.104 mL, 0.595 mmol) followed by methyl 4-(chloroformyl)butyrate (0.082 mL, 0.595 mmol). The mixture stirred at for 17 h then was poured into 1N HCl (5 mL) and was extracted with dichloromethane (3×5 mL). The combined organic layers were dried over sodium sulfate, filtered and concentrated under reduced pressure. The residue (82 mg, 0.12 mmol) was diluted with methanol (2 mL) and water (1 mL) and potassium carbonate (0.083 g, 0.60 mmol) was added. The mixture was heated to 60° C. for 26 h, then was further stirred art for 4 days. 1.5 mL of 1N NaOH was then added and the mixture was heated to 60° C. After 4.5 h of heating, the mixture was cooled to rt, acidified with 1N HCl and extracted with dichloromethane (3×10 mL). The organic layers were dried over sodium sulfate, filtered and concentrated under reduced pressure. The residue was purified by flash chromatography using a 0-5% methanol in dichloromethane gradient. The product did not elute, so the column was further washed with a 5-10% methanol in dichloromethane gradient with 0.1% acetic acid added. The fractions containing the title product were combined and concentrated to give a clear, colorless film (0.018 g, 0.033 mmol, 28% yield). LCMS: m/e 542.48 (M+H)⁺, 1.28 min (method 1A).

### Example 9

### Preparation of 4-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-4-oxobutanoic acid

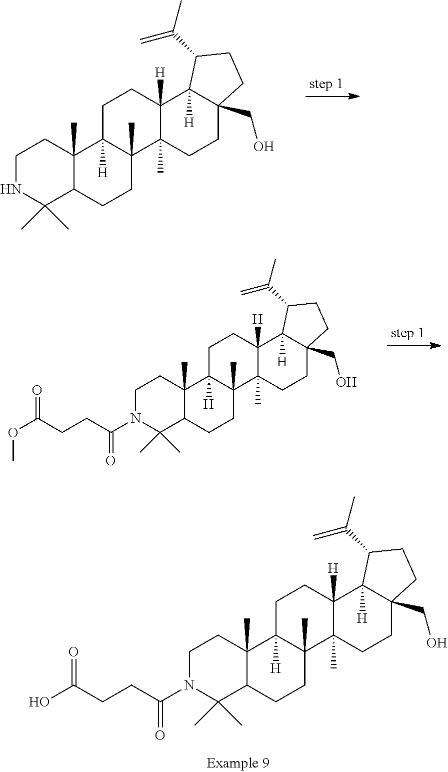

Step 1. Preparation of methyl 4-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-4-oxobutanoate

To a mixture of ((6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methanol (0.05 g, 0.117 mmol) in DCE (2 mL) was added Hunig's Base (0.061 mL, 0.351 mmol) followed by 3-carbomethoxypropionyl chloride (0.015 mL, 0.123 mmol). The mixture stirred at rt for 17 h then was poured into 1N HCl (4 mL) and extracted with dichloromethane (3×3 mL). The combined organic layers were dried over magnesium sulfate, filtered and concentrated under reduced pressure. The residue was purified by flash chromatography using a 0-30% ethyl acetate in hexanes gradient and a 12 g silica gel column. The fractions containing the product were combined and concentrated to give the title product (43.5 mg, 0.076 mmol, 65.2% yield) as a white solid. LCMS: m/e 542.48 (M+H)⁺, 1.83 min (method 1A).

Step 2. To a solution of 4-((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)-4-oxobutanoate (32 mg, 0.059 mmol) in DMF (2 mL) was added lithium hydroxide monohydrate (25 mg, 0.596 mmol). The mixture was diluted with water (1 mL) and stirred overnight at rt. After stirring the mixture for 24.5 h at rt, the reaction was quenched with 1 mL of 1N HCl and was concentrated under reduced pressure. The residue was purified by flash chromatography using a 0-5% methanol in dichloromethane gradient with 0.1% acetic acid on a 12 g silica gel column. The title product (0.028 g, 0.053 mmol, 90% yield) was isolated as a white foam. LCMS: m/e 528.49 (M+H)⁺, 1.33 min (method 1A).

### Example 10

### Preparation of 4-((6aR,6bR,8aS,11R,11aR,11bR,13aR,13bR)-8a-(hydroxymethyl)-4,4,6a,6b,13b-pentamethyl-11-(prop-1-en-2-yl)icosahydro-1H-cyclopenta[5,6]naphtho[2,1-f]isoquinoline-3-carbonyl)benzoic acid

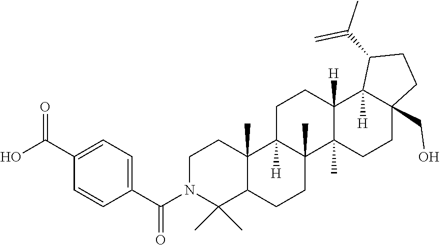

To a vial containing ((6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methanol (0.05 g, 0.117 mmol) was added terephthalic acid monomethyl ester chloride (0.026 g, 0.129 mmol). The mixture was diluted with DCE (2 mL) and Hunig's Base (0.061 mL, 0.351 mmol) and was stirred at rt overnight. The reaction was not complete, so an additional 0.015 g of terephthalic acid monomethyl ester chloride was added to the mixture and it was stirred at rt. After 4.5 h of stirring at rt, the reaction was still not complete so an additional 0.05 g of terephthalic acid monomethyl ester chloride was added and the mixture was warmed to 50° C. After 3 h of heating, the mixture was cooled to rt then stirred at rt for 3 days. The mixture was diluted with water (3 mL) and was extracted with dichloromethane (3×5 mL). The combined organic layers were dried over sodium sulfate, filtered and concentrated under reduced pressure. The crude residue was diluted with 1,4-dioxane (4 mL) and water (1 mL). Lithium hydroxide monohydrate was added and the mixture was heated to 75° C. After 2 h of heating, the mixture was cooled to rt and was diluted with 5 mL of 1N HCl and then was extracted with dichloromethane (3×7 mL). The organic layers were dried over sodium sulfate, filtered and concentrated under reduced pressure. The title product was crystallized and isolated from hot dioxane, methanol and water as an off-white solid (0.0325 g, 0.056 mmol, 48% yield). LCMS: m/e 576.49 (M+H)⁺, 2.00 min (method 3A).

### General Method A for Sulfonamide Synthesis

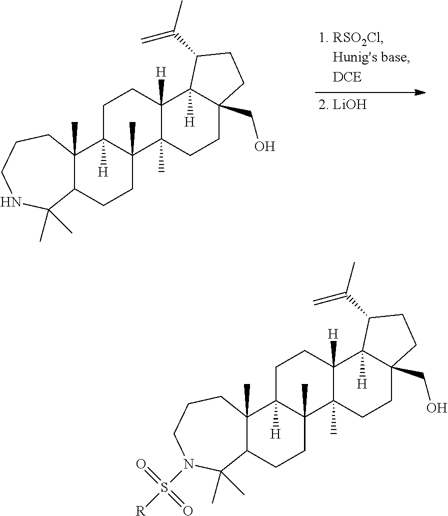

To a vial containing a solution of ((5aR,7aR,7bR,9aS,12R,12aR,12bR,14aR,14bR)-5,5,7a,7b,14b-pentamethyl-12-(prop-1-en-2-yl)icosahydrocyclopenta[7,8]phenanthro[2,1-c]azepin-9a(1H)-yl)methanol (440 mg, 1.00 mmol) in dichloroethane (22.0 mL) was added Hunig's base (0.517 mL, 4.00 mmol) to prepare a stock solution of the template. To separate vials containing the reactant sulfonyl chlorides (0.095 mmol, 1.05 eq.) was added 2.0 mL of the stock solution of template. Each vial was sealed and shaken vigorously overnight at room temperature. Once analysis showed the reactions were complete, they were concentrated, diluted with DMF (2.0 mL) and lithium hydroxide monohydrate (39.0 mg, 0.93 mmol) was added followed by 1.0 mL of water. The vials were shaken vigorously overnight at rt. The mixtures were diluted with a small amount of methanol and shaken for another day at rt, then concentrated and diluted with methanol (1.2 mL) and DMSO (0.4 mL). The mixtures were shaken vigorously and 50 μL of 37% HCl was added. The mixtures were sonicated to dissolve the solids and purified by prep HPLC to give the sulfonamide products.

### Example 11

### Preparation of 5-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)furan-2-carboxylic acid

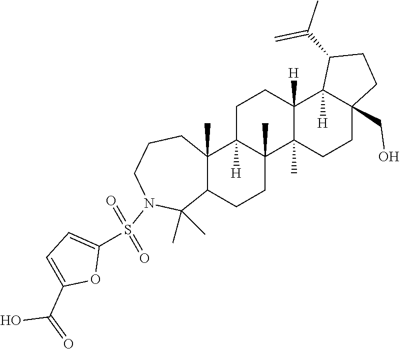

The preparation of 5-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)furan-2-carboxylic acid was accomplished using the general method A for sulfonamide formation. 5-(chlorosulfonyl)furan-2-carboxylic acid methyl ester (0.02 g) was used as the reactant sulfonyl chloride. The material was purified using prep HPLC purification method 1. LCMS: m/e 616.38 (M+H)⁺, 2.92 min (method 5A).

### Example 12

### Preparation of (E)-3-(4-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)phenyl)acrylic acid

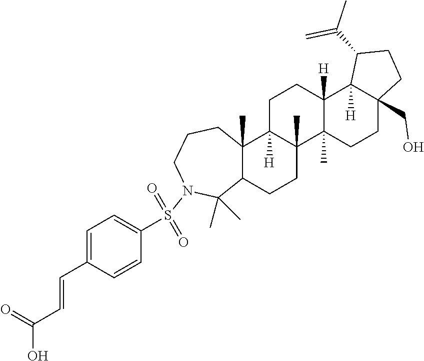

The preparation of (E)-3-(4-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)phenyl)acrylic acid was accomplished using the general method A for sulfonamide formation. (E)-3-(4-(chlorosulfonyl)phenyl)acrylic acid (0.0235 g) was used as the reactant sulfonyl chloride. The material was purified using prep HPLC purification method 1. LCMS: m/e 652.47 (M+H)⁺, 3.05 min (method 5A).

### Example 13

### Preparation of 3-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)benzoic acid

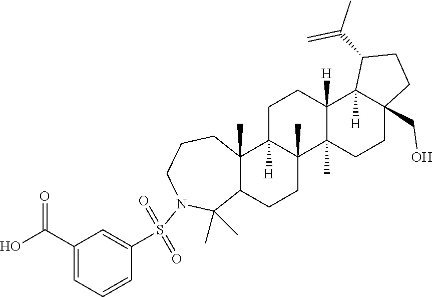

The preparation of 3-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)benzoic acid was accomplished using the general method A for sulfonamide formation. 3-(chlorosulfonyl)benzoic acid (0.021 g) was used as the reactant sulfonyl chloride. The material was purified using prep HPLC purification method 1. LCMS: m/e 626.42 (M+H)⁺, 2.89 min (method 5A).

### Example 14

### Preparation of 4-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)benzoic acid

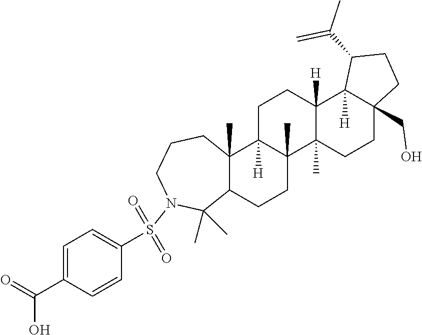

The preparation of 4-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)benzoic acid was accomplished using the general method A for sulfonamide formation. 4-(chlorosulfonyl)benzoic acid (0.021 g) was used as the reactant sulfonyl chloride. The material was purified using prep HPLC purification method 1. LCMS: m/e 626.40 (M+H)⁺, 2.82 min (method 5A).

### Example 15

### Preparation of 2-(4-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)phenyl)acetic acid

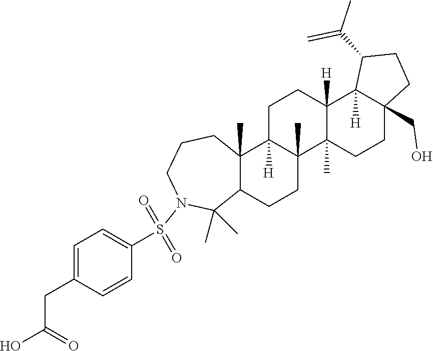

The preparation of 2-(4-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)phenyl)acetic acid was accomplished using the general method A for sulfonamide formation. 2-(4-(chlorosulfonyl)phenyl)acetic acid (0.022 g) was used as the reactant sulfonyl chloride. The material was purified using prep HPLC purification method 1. LCMS: m/e 640.45 (M+H)⁺, 3.06 min (method 5A).

### Example 16

### Preparation of 5-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)-2-methylbenzoic acid

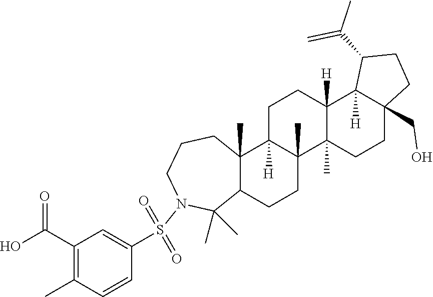

The preparation of 5-(((7aR,7bR,9aS,12R,12aR,12bR,14aR,14bR)-9a-(hydroxymethyl)-5,5,7a,7b,14b-pentamethyl-12-(prop-1-en-2-yl)icosahydrocyclopenta[7,8]phenanthro[2,1-c]azepin-4(1H)-yl)sulfonyl)-2-methylbenzoic acid was accomplished using the general method A for sulfonamide formation. 5-(chlorosulfonyl)-2-methylbenzoic acid (0.022 g) was used as the reactant sulfonyl chloride. The material was purified using prep HPLC purification method 1. LCMS: m/e 640.41 (M+H)⁺, 2.99 min (method 5A).

### General Method B for Sulfonamide Synthesis

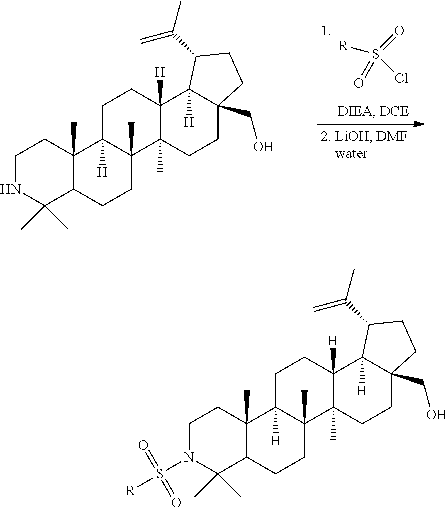

The sulfonamide synthesis was run using the same reaction conditions as the amide synthesis method B above and the same stock solution of template. Instead of using acid chloride reactants, sulfonamides were prepared using the reactant sulfonyl chlorides. Workup and purification was run in parallel to the amide series following the same conditions.

### Example 17

### Preparation of 3-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)benzoic acid

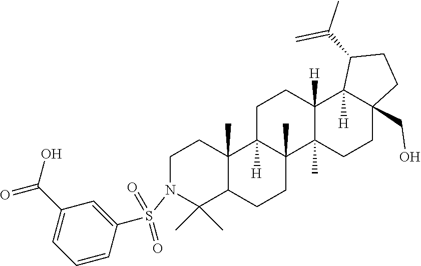

The preparation of 3-(((6aR,6bR,8aS,11R,11aR,11bR,13aR,13bR)-8a-(hydroxymethyl)-4,4,6a,6b,13b-pentamethyl-11-(prop-1-en-2-yl)icosahdro-3H-cyclopenta[5,6]naphtho[2,1-f]isoquinolin-3-yl)sulfonyl)benzoic acid was accomplished using the general method B for sulfonamide synthesis. 3-(chlorosulfonyl)benzoic acid was used as the reactant sulfonyl chloride. The material was purified using prep HPLC purification method 4. LCMS: m/e 612.26 (M+H)⁺, 9.02 min (method 6A).

### Example 18

### Preparation of (E)-3-(4-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)phenyl)acrylic acid

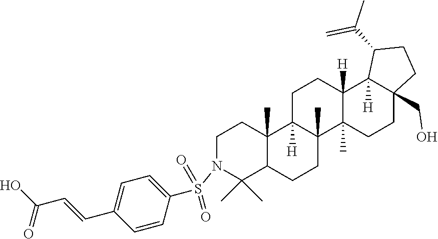

The preparation of (E)-3-(4-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)phenyl)acrylic acid was accomplished using the general method B for sulfonamide synthesis. (E)-3-(4-(chlorosulfonyl)phenyl)acrylic acid was used as the reactant sulfonyl chloride. The material was purified using prep HPLC purification method 4. LCMS: m/e 638.35 (M+H)⁺, 9.58 min (method 6A).

### Example 19

### Preparation of 2-(4-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)phenyl)acetic acid

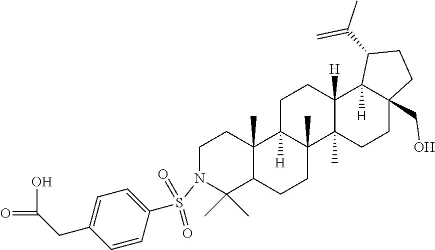

The preparation of 2-(4-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)phenyl)acetic acid was accomplished using the general method B for sulfonamide synthesis. 2-(4-(chlorosulfonyl)phenyl)acetic acid was used as the reactant sulfonyl chloride. The material was purified using prep HPLC purification method 4. LCMS: m/e 626.33 (M+H)⁺, 9.24 min (method 6A).

### Example 20

### Preparation of 4-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)benzoic acid

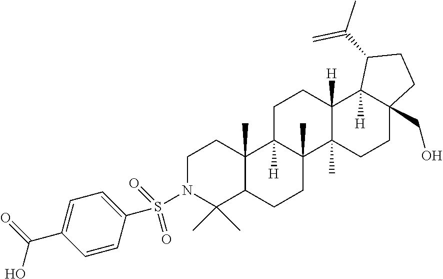

The preparation of 4-(((6aR,6bR,8aS,11R,11aR,11bR,13aR,13bR)-8a-(hydroxymethyl)-4,4,6a,6b,13 b-pentamethyl-11-(prop-1-en-2-yl)icosahydro-3H-cyclopenta[5,6]naphtho[2,1-f]isoquinolin-3-yl)sulfonyl)benzoic acid was accomplished using the general method B for sulfonamide synthesis. 4-(chlorosulfonyl)benzoic acid was used as the reactant sulfonyl chloride. The material was purified using prep HPLC purification method 4. LCMS: m/e 612.28 (M+H)⁺, 8.94 min (method 6A).

### Example 21

### Preparation of 3-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)thiophene-2-carboxylic acid

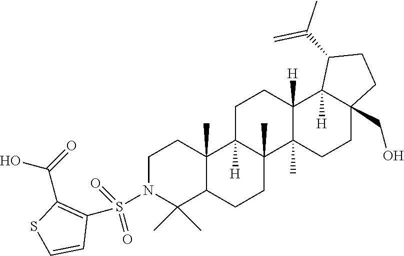

The preparation of 3-(((6aR,6bR,8aS,11R,11aR,11bR,13aR,13bR)-8a-(hydroxymethyl)-4,4,6a,6b,13b-pentamethyl-11-(prop-1-en-2-yl)icosahydro-3H-cyclopenta[5,6]naphtho[2,1-f]isoquinolin-3-yl)sulfonyl)thiophene-2-carboxylic acid was accomplished using the general method B for sulfonamide synthesis. Methyl 3-(chlorosulfonyl)thiophene-2-carboxylate was used as the reactant sulfonyl chloride. The material was purified using prep HPLC purification method 4. LCMS: m/e 618.26 (M+H)⁺, 8.88 min (method 6A).

### SECTION 2—EXAMPLES

### LC/MS Methods

### Method 1B

Start % B=20; Final % B=100; gradient time=3 min
Flow Rate=4 mL/min
Wavelength=220 nm
Solvent A=10% MeOH—90% H₂O—0.1% TFA
Solvent B=90% MeOH—10% H₂O—0.1% TFA
Column=Xbridge Phenyl 4.6×50 mm S5

### Method 2B

Start % B=20; Final % B=100; gradient time=2 min
Flow Rate=0.8 mL/min
Wavelength=254 nm
Solvent A=10% MeOH—90% H₂0—0.1% TFA
Solvent B=90% MeOH—10% H₂0—0.1% TFA
Column=Xbridge Phenyl 2.1×50 mm 2.5 μm

### Method 3B

Start % B=0; final % B=100; gradient time=2 min
Flow Rate=1 mL/min
Wavelength=220 nm
Solvent A=10% MeOH—90% H₂0—0.1% TFA
Solvent B=90% MeOH—10% H₂0—0.1% TFA
Column=PHENOMENEX-LUNA 2.0×30 mm 3 μm

### Method 4B

Start % B=10; Final % B=100; gradient time=2 min
Flow Rate=1 mL/min
Wavelength=220 nm
Solvent A=10% MeOH—90% H₂0—0.1% TFA
Solvent B=90% MeOH—10% H₂0—0.1% TFA
Column=PHENOMENEX-LUNA 2.0×30 mm 3 μm

### Method 5B

Start % B=20; Final % B=100; gradient time=2 min
Flow Rate=4 mL/min
Wavelength=220 nm
Solvent A=10% MeOH—90% H₂0—0.1% TFA
Solvent B=90% MeOH—10% H₂0—0.1% TFA
Column 3=Xbridge Phenyl 4.6×50 mm S5

### Method 6B

Start % B=10; Final % B=100; gradient time=2 min
Flow Rate=0.8 mL/min
Wavelength=220 nm
Solvent A=10% MeOH—90% H₂0—0.1% TFA
Solvent B=90% MeOH—10% H₂0—0.1% TFA
Column 4=Xbridge C8 2.1×50 mm 2.5 μm

### Method 7B

Start % B=30; Final % B=100; gradient Time=2 min
Flow Rate=1 ml/min
Wavelength=220 nm
Solvent A=10% MeOH—90% H₂O—0.1% TFA
Solvent B=90% MeOH—10% H₂O—0.1% TFA
Column=Phenomenex-Luna 2.0×30 mm 3 μm

### Method 8B

Start % B=20; Final % B=100 over 2 minute gradient, hold at 100% B
Flow Rate=0.8 mL/min
Wavelength=220 nm
Solvent A=90% water, 10% methanol, 0.1% TFA
Solvent B=10% water, 90% methanol, 0.1% TFA
Column=Xbridge Phenyl 2.1×50 mm 2.5 μM

### Method 9B

Start % B=20; Final % B=100 over 2 minute gradient, hold at 100% B
Flow Rate=1.0 mL/min
Wavelength=220 nm
Solvent A=90% water, 10% methanol, 0.1% TFA
Solvent B=10% water, 90% methanol, 0.1% TFA
Column=Phenomenex Luna 2.0×30 mm 3 μM

### Method 10B

Start % B=0; Final % B=100 over 2 minute gradient, hold at 100% B
Flow Rate=1.0 mL/min
Wavelength=220 nm
Solvent A=95% water, 5% methanol, 10 mM ammonium acetate
Solvent B=5% water, 95% methanol, 10 mM ammonium acetate
Column=Phenomenex Luna 2.0×30 mm C18, 3 μM

### Method 11B

Start % B=0; Final % B=100 over 4 minute gradient, hold at 100% B
Flow Rate=0.8 mL/min
Wavelength=220 nm
Solvent A=90% water, 10% methanol, 0.1% TFA
Solvent B=10% water, 90% methanol, 0.1% TFA
Column=Phenomenex C18 2.0×50 mm 3 μM

### Method 12B

Start % B=20; Final % B=100 over 4 minute gradient, hold at 100% B
Flow Rate=0.8 mL/min
Wavelength=220 nm
Solvent A=90% water, 10% methanol, 0.1% TFA
Solvent B=10% water, 90% methanol, 0.1% TFA
Column=Phenomenex C18 2.0×50 mm 3 μM

### Preparative HPLC

All of the compounds prepared in this Section 2 were purified using the following HPLC method.
Start % B=30; Final % B=100; Gradient time=8 min
Flow Rate=25 mL/min

### ELSD as External Detector

Solvent A=10 mM Ammonium Acetate in 95:5 H₂O/CH₃CN
Solvent B=10 mM Ammonium Acetate in 5:95 H₂O/CH₃CN
Column=Xbridge PREP phenyl OBD C18 19×100 mm 5 μm

### Example 22

### Preparation of (4aR,6aR,6bR,8aS,11R,11aR,11bR,13aR,13bR)-3-(4-carboxyphenyl)-4,4,6a,6b,13b-pentamethyl-2-oxo-11-(prop-1-en-2-yl)icosahydro-8aH-cyclopenta[5,6]naphtho[2,1-f]isoquinoline-8a-carboxylic acid

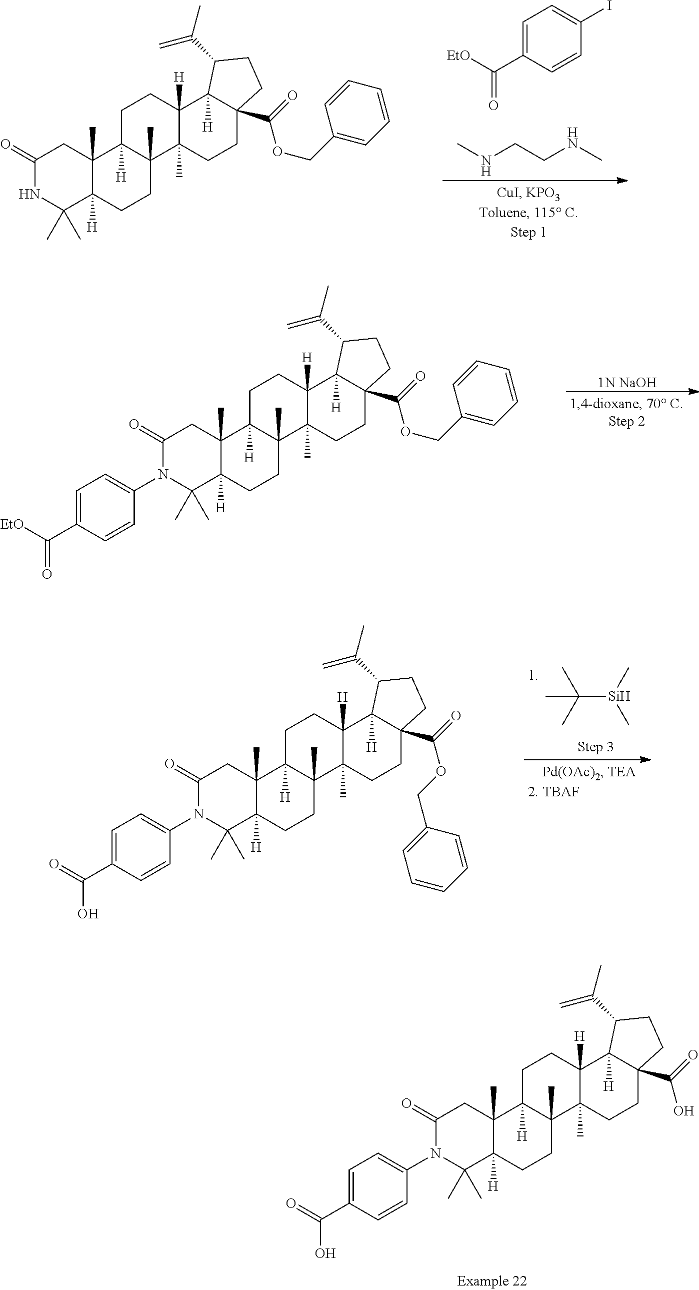

Step 1. Preparation of Benzyl(4aR,6aR,6bR,8aS,11R,11aR,11bR,13aR,13bR)-3-(4-(ethoxycarbonyl)phenyl)-4,4,6a,6b,13b-pentamethyl-2-oxo-11-(prop-1-en-2-yl)icosahydro-8aH-cyclopenta[5,6]naphtho[2,1-f]isoquinoline-8a-carboxylate.

To a mixture of benzyl (4aR,6aR,6bR,8aS,11R,11aR,11bR,13aR,13bR)-4,4,6a,6b,13b-pentamethyl-2-oxo-11-(prop-1-en-2-yl)icosahydro-8aH-cyclopenta[5,6]naphtho[2,1-f]isoquinoline-8a-carboxylate (66 mg, 0.121 mmol), ethyl 4-iodobenzoate (36.7 mg, 0.133 mmol), potassium metaphosphate (31.4 mg, 0.266 mmol) and copper(I) iodide (0.410 μL, 0.012 mmol) in a small sealable tube was added N,N′-dimethylethylenediamine (2.132 mg, 0.024 mmol) and toluene (2 mL). The slightly turbid, greenish blue suspension was purged with nitrogen several times and placed in an oil bath at 115° C. for 24 hours. The mixture was cooled to rt and was purified by silica gel chromatography eluted with mixtures of ethyl acetate and hexanes. The fractions containing the product were combined and concentrated to give the title product (9.9 mg, 11.8%). LC/MS: m/e 694.45 (M+H)⁺, 4.41 min (method 1B). ¹H NMR (400 MHz, CDCl₃) δ 8.15-7.94 (m, 2H), 7.51-7.24 (m, 3H), 5.27-5.03 (m, 2H), 4.72 (d, J=1.8 Hz, 1H), 4.59 (d, J=1.3 Hz, 1H), 4.39 (q, J=7.1 Hz, 2H), 3.44-3.28 (m, 2H), 3.03 (td, J=10.9, 4.7 Hz, 1H), 2.40-2.19 (m, 2H), 2.01-1.81 (m, 2H), 1.80-1.66 (m, 1H), 1.68 (s, 3H), 1.66-1.57 (m, 2H), 1.54-1.37 (m, 10H), 1.41 (t, J=7.05 Hz, 3H), 1.34 (dd, J=13.5, 3.4 Hz, 2H), 1.31-1.21 (m, 3H), 1.28 (s, 3H), 1.23 (s, 3H), 1.17 (d, J=13.1 Hz, 1H), 1.13-0.96 (m, 1H), 1.09 (s, 3H), 1.00 (s, 3H), 0.85 (s, 3H).

Step 2. Preparation of 4-((4aR,6aR,6bR,8aS,11R,11aR,11bR,13aR,13bR)-8a-((benzyloxy)carbonyl)-4,4,6a,6b,13b-pentamethyl-2-oxo-11-(prop-1-en-2-yl)icosahydro-3H-cyclopenta[5,6]naphtho[2,1-f]isoquinolin-3-yl)benzoic acid

To a solution of benzyl (4aR,6aR,6bR,8aS,11R,11aR,11bR,13aR,13bR)-3-(4-(ethoxycarbonyl)phenyl)-4,4,6a,6b,13b-pentamethyl-2-oxo-11-(prop-1-en-2-yl)icosahydro-8aH-cyclopenta[5,6]naphtho[2,1-f]isoquinoline-8a-carboxylate (0.015 g, 0.022 mmol) in 1,4-dioxane (2 mL) was added 1N of NaOH (0.086 ml, 0.086 mmol) at rt. The resulted solution was stirred at 70° C. for 2 h. The reaction mixture was cooled to rt, then purified by Prep HPLC. The fractions containing title compound were combined and concentrated under reduced pressure to yield white solid (14 mg, 97%). MS m/e 666.38 (M+H)⁺, 3.29 min (method 5B).

Step 3. To a solution of 4-((4aR,6aR,6bR,8aS,11R,11aR,11bR,13aR,13bR)-8a-((benzyloxy)carbonyl)-4,4,6a,6b,13b-pentamethyl-2-oxo-11-(prop-1-en-2-yl)icosahydro-3H-cyclopenta[5,6]naphtho[2,1-f]isoquinolin-3-yl)benzoic acid (14 mg, 0.021 mmol) in DCE (3.5 mL) was added TEA (4.69 μL, 0.034 mmol), tert-butyldimethylsilane (4.89 mg, 0.042 mmol) and palladium(II) acetate (1.180 mg, 5.26 μmol). The mixture was flushed with nitrogen and heated to 60° C. After 2.5 h the mixture was cooled to rt. LC/MS indicated the reaction was complete. The mixture was filtered through a pad of celite to remove the solids and was concentrated under reduced pressure. The residue was diluted with 5 mL of THF. To the cloudy solution was added tetrabutylammonium fluoride hydrate (0.152 g, 0.545 mmol). The mixture was stirred at rt for 2 hours, LC/MS indicated the reaction was complete. The mixture was diluted with water (15 mL) and extracted with ethyl acetate (3×15 mL). The combined organic layers were dried over sodium sulfate, filtered and concentrated under reduced pressure. The residue was purified by prep HPLC to give (4aR,6aR,6bR,8aS,11R,11aR,11bR,13aR,13bR)-3-(4-carboxyphenyl)-4,4,6a,6b,13b-pentamethyl-2-oxo-11-(prop-1-en-2-yl)icosahydro-8aH-cyclopenta[5,6]naphtho[2,1-f]isoquinoline-8a-carboxylic acid as a white solid (7.0 mg, 54.9%). MS m/e 576.36 (M+H)⁺, 2.94 min (method 5B). ¹H NMR (400 MHz, methanol-d₄) δ 8.11-7.94 (m, 2H), 7.35-7.18 (m, 2H), 4.66 (s, 1H), 4.54 (s, 1H), 3.45 (d, J=11.8 Hz, 1H), 3.34 (m, 1H), 2.99 (td, J=10.7, 4.8 Hz, 1H), 2.43-2.29 (m, 1H), 2.23 (d, J=12.3 Hz, 1H), 1.98-1.81 (m, 2H), 1.70 (d, J=13.3 Hz, 1H), 1.66 (s, 3H), 1.63-1.00 (m, 15H), 1.24 (s, 3H), 1.21-1.15 (m, 3H), 1.12 (s, 3H), 1.06-1.00 (m, 6H).

### Example 23

### Preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid

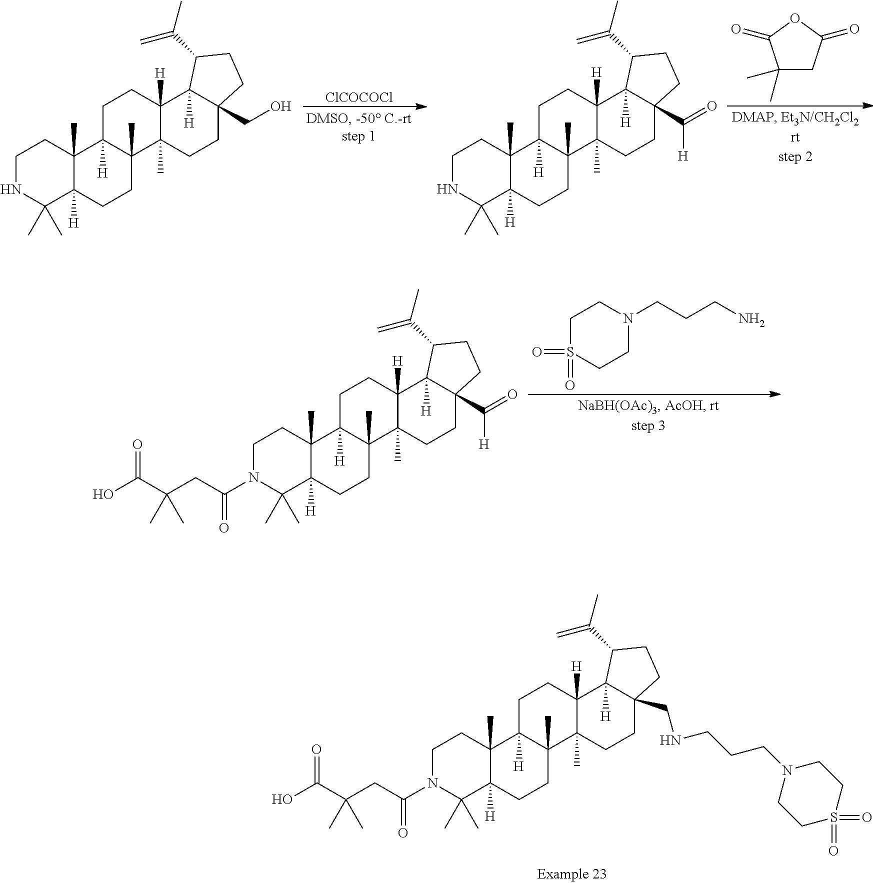

Step 1: Preparation of (4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinoline-8a-carbaldehyde.

To a solution of oxalyl chloride (0.421 ml, 0.842 mmol) in dichloromethane (5.0 ml) at −70° C. was added dropwise a solution of DMSO (0.075 ml, 1.052 mmol) in dichloromethane (5 ml). The mixture was warmed to −50° C. and a solution of ((4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methanol (300 mg, 0.701 mmol) in dichloromethane (2.0 ml) was added dropwise. The mixture was stirred for 15 min at −50° C. then Et₃N (0.293 ml, 2.104 mmol) was added dropwise and the mixture was slowly warmed to rt. The reaction mixture was diluted with dichloromethane (50 ml) and was washed with H₂O (2×50 ml) followed by brine (50 ml). The organic layer was dried over sodium sulfate, filtered and concentrated in vacuo. The crude product (305 mg) was used with no additional purification. MS m/e 426.24 (M+H)⁺, 2.32 min (method 2B).

Step 2: Preparation of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid. (4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinoline-8a-carbaldehyde (70 mg, 0.164 mmol) and 3,3-dimethyldihydrofuran-2,5-dione (42.1 mg, 0.329 mmol) in dichloromethane (3.0 mL) was added TEA (0.023 mL, 0.164 mmol) followed by DMAP (20.09 mg, 0.164 mmol). The mixture was stirred overnight, then diluted with ethyl acetate (10 mL) and washed with water (10 ml). The organic phase was collected and dried over sodium sulfate. The filtrate was evaporated after filtration and the resulting solid was used in the next step with no additional purification. MS m/e 554.23 (M+H)⁺, 2.80 min (method 2B).

Step 3: To a solution of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid (91 mg, 0.164 mmol) in DCE (2 mL) was added acetic acid (0.0940, 1.640 μmol) and 4-(3-aminopropyl)thiomorpholine 1,1-dioxide (63.1 mg, 0.328 mmol). The mixture was stirred for 10 minutes at which point it became clear then was stirred at rt for 2 h. To the the mixture was added sodium triacetoxyborohydride (174 mg, 0.820 mmol) and it was stirred overnight at rt. The mixture was diluted with 7 mL of sat. sodium bicarbonate and was extracted with dichloromethane (3×7 mL). The combined organic layers were dried with over sodium sulfate, filtered and concentrated under reduced pressure. The crude product was purified by prep HPLC. The fractions containing product were collected and concentrated in vacuo to give 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as a white solid (7 mg, 4.09%). MS m/e 730.23 (M+H)⁺, 2.29 min (method 2B). ¹H NMR (400 MHz, CDCl₃) δ 4.70 (s, 1H), 4.62 (s, 1H), 3.56-3.43 (m, 1H), 3.39-3.25 (m, 1H), 3.21-2.95 (m, 10H), 2.87-2.33 (m, 6H), 2.29-2.10 (m, 1H), 2.00-0.88 (m, 48H).

### Example 24

### Preparation of 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-3,3-dimethyl-5-oxopentanoic acid

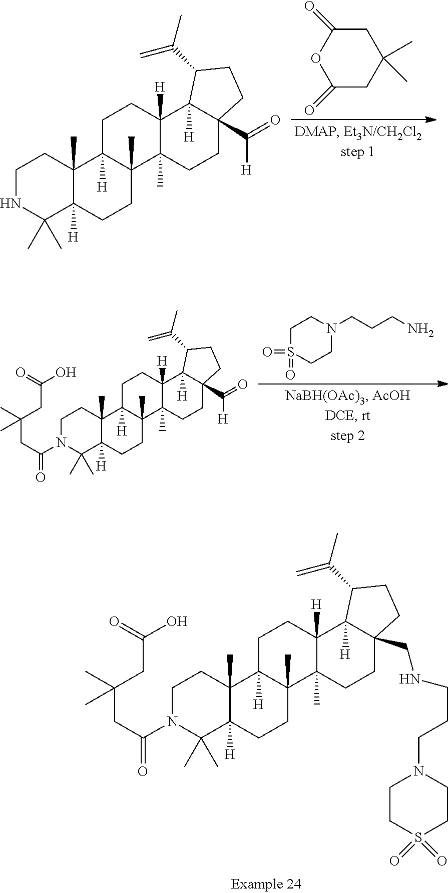

Step 1: Preparation of 5-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-3,3-dimethyl-5-oxopentanoic acid. The title compound was prepared in 100% yield following the procedure described above for preparation of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, using 4,4-dimethyldihydro-2H-pyran-2,6(3H)-dione instead of 3,3-dimethyldihydrofuran-2,5-dione as the reactant. MS: m/e 568.12 (M+H)⁺, 2.79 min (method 2B).

Step 2: 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-3,3-dimethyl-5-oxopentanoic acid was prepared in 19.67% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid using 5-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-3,3-dimethyl-5-oxopentanoic acid instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 744.32 (M+H)⁺, 2.31 min (method 2B). ¹H NMR (400 MHz, CDCl₃-d) δ 4.75-4.65 (m, 1H), 4.62 (s, 1H), 3.68-3.53 (m, 1H), 3.41-3.27 (m, 1H), 3.05 (d, J=8.5 Hz, 8H), 2.94-2.75 (m, 3H), 2.66-2.56 (m, 2H), 2.50-2.24 (m, 6H), 2.05-0.75 (m, 48H).

### Example 25

### 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2,2-dimethyl-5-oxopentanoic acid

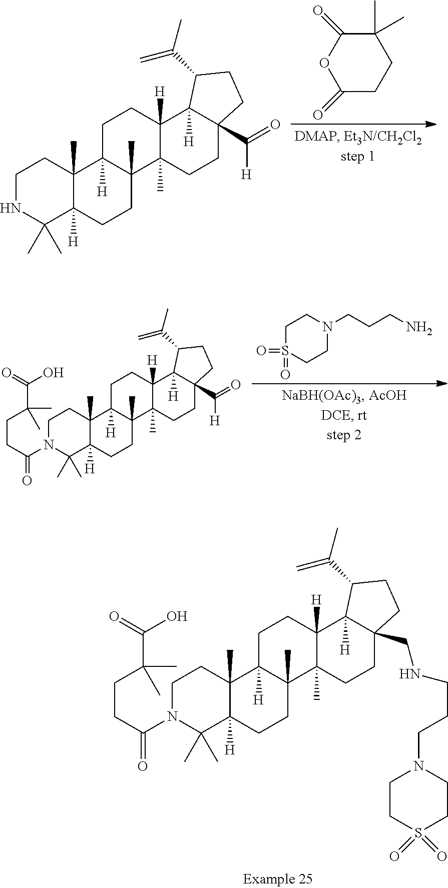

Step 1: Preparation of 5-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-5-oxopentanoic acid.

The title compound was prepared in 11.87% yield following the procedure described above for preparation of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, using 3,3-dimethyldihydro-2H-pyran-2,6(3H)-dione instead of 3,3-dimethyldihydrofuran-2,5-dione as the reactant. MS: m/e 568.55 (M+H)⁺, 2.74 min (method 2B). ¹H NMR (400 MHz, CDCl₃) δ 9.68 (d, J=1.5 Hz, 1H), 4.78 (d, J=1.8 Hz, 1H), 4.70-4.57 (m, 1H), 3.51-3.36 (m, 1H), 3.34-3.20 (m, 1H), 2.90 (td, J=11.1, 5.9 Hz, 1H), 2.41-2.23 (m, 2H), 2.16-2.01 (m, 2H), 1.72 (s, 3H), 1.97-1.61 (m, 6H), 1.48 (s, 3H), 1.40 (s, 3H), 1.23 (s, 6H), 1.00 (s, 3H), 0.97 (s, 3H), 0.96 (s, 3H), 1.56-0.89 (m, 16H).

Step 2: 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2,2-dimethyl-5-oxopentanoic acid was prepared in 21.75% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using 5-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-5-oxopentanoic acid instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 744.63 (M+H)⁺, 2.28 min (method 2B). ¹H NMR (400 MHz, CDCl₃) δ 4.71 (s, 1H), 4.61 (s, 1H), 3.42 (d, J=13.8 Hz, 1H), 3.27 (br. s., 1H), 3.05 (d, J=10.3 Hz, 8H), 2.93-2.75 (m, 3H), 2.63 (t, J=6.5 Hz, 2H), 2.43 (d, J=12.0 Hz, 2H), 2.27 (d, J=7.0 Hz, 2H), 1.69 (s, 3H), 1.48 (s, 3H), 1.40 (s, 3H), 1.17 (s, 6H), 1.06 (s, 3H), 1.00 (s, 3H), 0.96 (s, 3H), 2.06-0.90 (m, 26H).

### Example 26

### Preparation of 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid

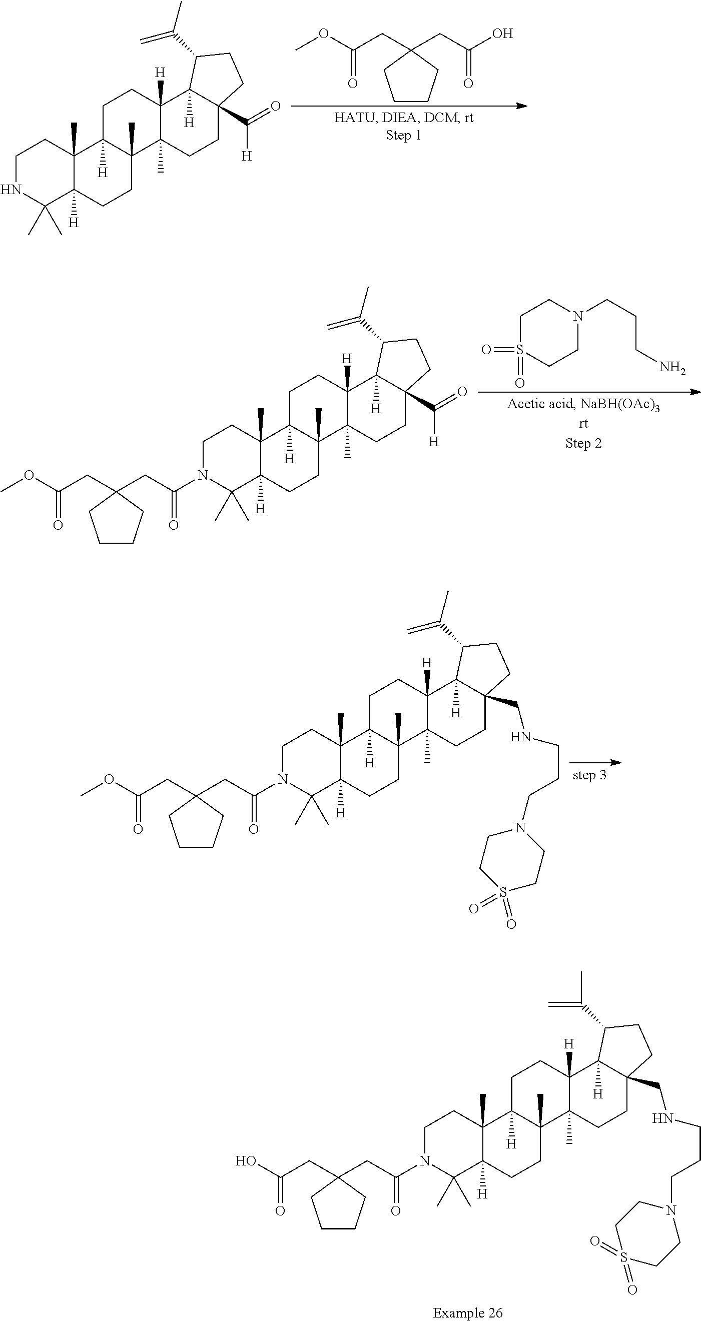

### Preparation of 2-(1-(2-methoxy-2-oxoethyl)cyclopentyl)acetic acid

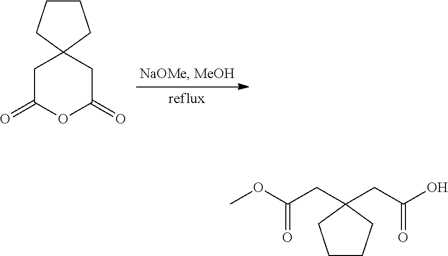

8-oxaspiro[4.5]decane-7,9-dione (2.8 g, 16.65 mmol) was dissolved in sodium methoxide in methanol (66.6 ml, 33.3 mmol) then was brought to reflux for 3 hours. The resulting reaction mixture was cooled to room temperature then poured into an ice-water mixture and adjusted pH=2. The mixture was extracted with ether (2×100 ml) and the combined organic layer was dried over sodium sulfate, filtered and concentrated under reduced pressure. The expected product was isolated as a light yellow oil (2.6 g, 70%). ¹H NMR (400 MHz, CDCl₃) δ 3.69 (s, 3H), 2.61 (s, 4H), 1.88-1.27 (m, 8H).

Step 1: Preparation of methyl 2-(1-(2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate.

The title compound was prepared in 48.5% yield following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoate, using 2-(1-(2-methoxy-2-oxoethyl)cyclopentyl)acetic acid instead of 4-tert-butoxy-3,3-dimethyl-4-oxobutanoic acid as the reactant. MS: m/e 608.46 (M+H)⁺, 3.35 min (method 4B). ¹H NMR (400 MHz, CDCl₃) δ 9.68 (s, 1H), 4.78 (d, J=1.8 Hz, 1H), 4.69-4.60 (m, 1H), 3.67-3.57 (m, 3H), 3.53-3.40 (m, 1H), 3.31-3.15 (m, 1H), 2.90 (td, J=11.1, 5.9 Hz, 1H), 2.73-2.42 (m, 4H), 2.21-0.73 (m, 48H).

Step 2: Preparation of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate

The title compound was prepared in 77.5% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using methyl 2-(1-(2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 784.70 (M+H)⁺, 2.50 min (method 4B).

Step 3: To a solution of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate (90 mg, 0.115 mmole) in 1,4-dioxane (3 ml) was added a 1N sodium hydroxide solution (0.5 ml, 0.500 mmol). The reaction was heated to 63° C. for 4 hrs. then 25 mg of solid sodium hydroxide was added to the solution and the mixture was again heated for an additional 3 hours. The mixture was cooled to rt and purified by prep HPLC. The fractions containing product were collected and concentrated in vacuo to give 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid as a white solid (24 mg, 27.0%). MS m/e 770.57 (M+H)⁺, 2.22 min (method 4B). ¹H NMR (400 MHz, CDCl₃) δ 4.70 (s, 1H), 4.63 (s, 1H), 3.65-3.53 (m, 1H), 3.42-3.29 (m, 1H), 3.12-2.90 (m, 11H), 2.68-2.33 (m, 8H), 1.69 (s, 3H), 1.54 (s, 3H), 1.42 (s, 3H), 1.06 (s, 3H), 0.99 (s, 3H), 0.98 (s, 3H), 1.95-0.89 (m, 32H).

### Example 27

### Preparation of 2-(3-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)phenyl)acetic acid

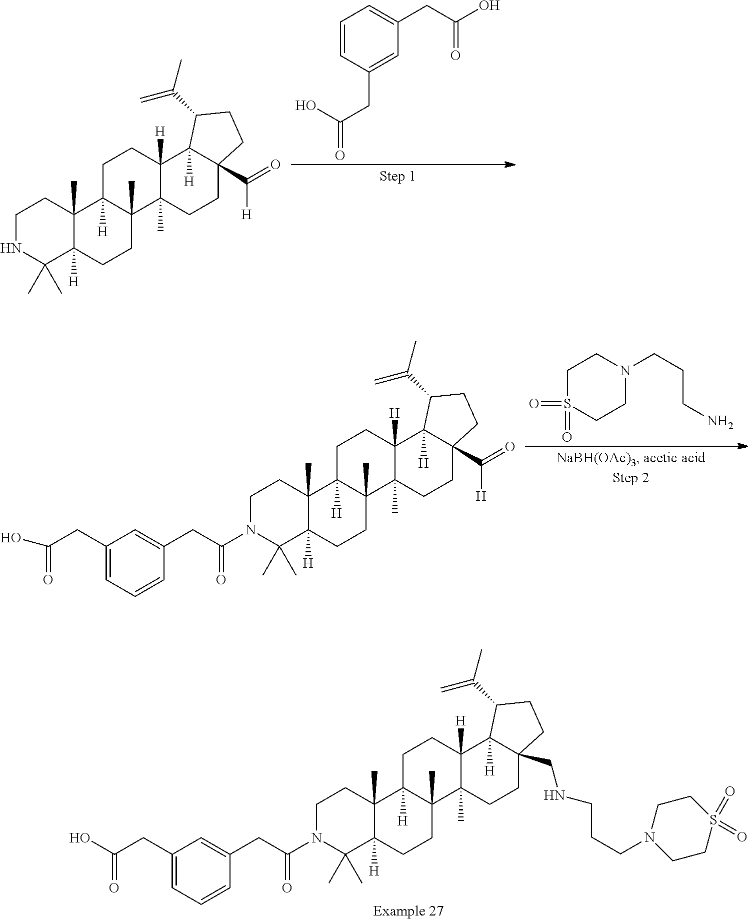

Step 1: Preparation of 2-(3-(2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)phenyl)acetic acid

The title compound was prepared in 70.7% yield following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoate, using 1,3-phenylenediacetic acid instead of 4-tert-butoxy-3,3-dimethyl-4-oxobutanoic acid as the reactant. MS: m/e 602.39 (M+H)⁺, 1.69 min (method 4B).

Step 2: 2-(3-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)phenyl)acetic acid was prepared in 9.75% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using 2-(3-(2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)phenyl)acetic acid instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 778.55 (M+H)⁺, 2.11 min (method 4B). ¹H NMR (400 MHz, CDCl₃) δ 7.26-7.20 (m, 1H), 7.16 (d, J=7.3 Hz, 1H), 7.13 (s, 1H), 7.03 (d, J=7.5 Hz, 1H), 4.69 (s, 1H), 4.62 (s, 1H), 3.66 (br. s., 2H), 3.52 (s, 2H), 3.45-3.40 (m, 1H), 3.30-3.21 (m, 1H), 3.09-2.85 (m, 11H), 2.66-2.32 (m, 4H), 1.99-1.74 (m, 5H), 1.68 (s, 3H), 1.53 (s, 3H), 1.42 (s, 3H), 1.02 (s, 3H), 0.97 (s, 3H), 0.88 (s, 3H), 1.73-0.84 (m, 19H).

### Example 28

### Preparation of 4-(3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxopropyl)benzoic acid

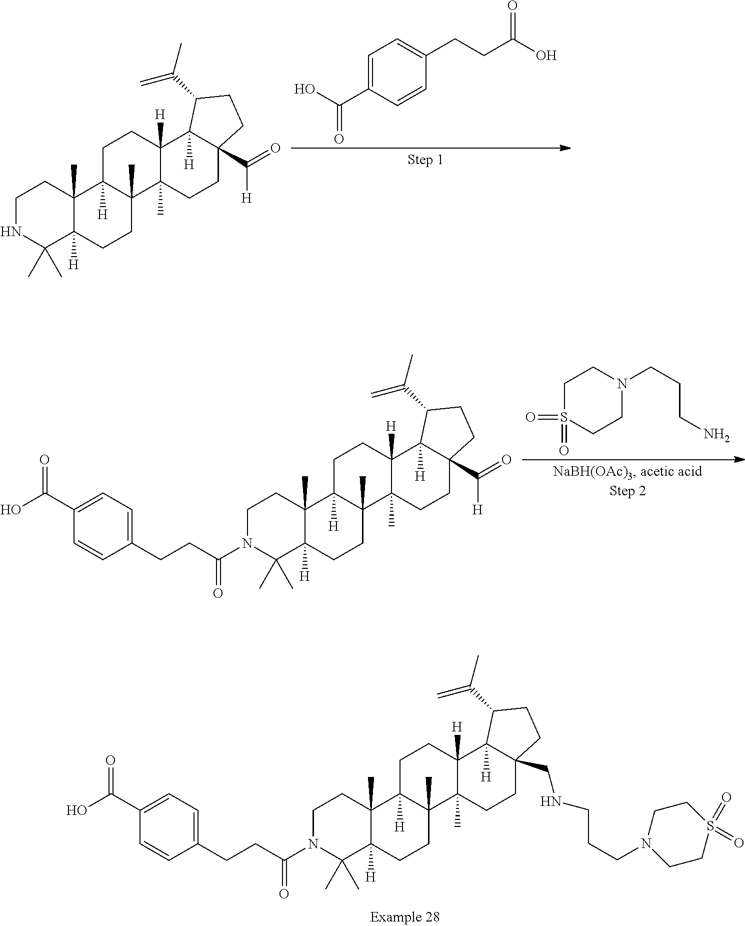

Step 1: Preparation of 4-(3-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxopropyl)benzoic acid.

The title compound was prepared in 70.0% yield following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoate, using 4-(2-carboxyethyl)benzoic acid instead of 4-tert-butoxy-3,3-dimethyl-4-oxobutanoic acid as the reactant. MS: m/e 602.39 (M+H)⁺, 1.85 min (method 4B).

Step 2: 4-(3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxopropyl)benzoic acid was prepared in 4.2% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using 4-(3-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxopropyl)benzoic acid instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 778.55 (M+H)⁺, 2.16 min (method 4B). ¹H NMR (400 MHz, CDCl₃) δ 7.92 (d, J=8.0 Hz, 2H), 7.25 (d, J=8.3 Hz, 2H), 4.71 (s, 1H), 4.63 (br. s., 1H), 3.39-2.80 (m, 14H), 2.72-2.33 (m, 7H), 2.02-1.79 (m, 5H), 1.69 (s, 3H), 1.48 (s, 3H), 1.38 (s, 3H), 1.04 (s, 3H), 0.96 (s, 3H), 0.90 (s, 3H), 1.75-0.87 (m, 19H).

### Example 29

### Preparation of 4-((E)-3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxoprop-1-en-1-yl)benzoic acid

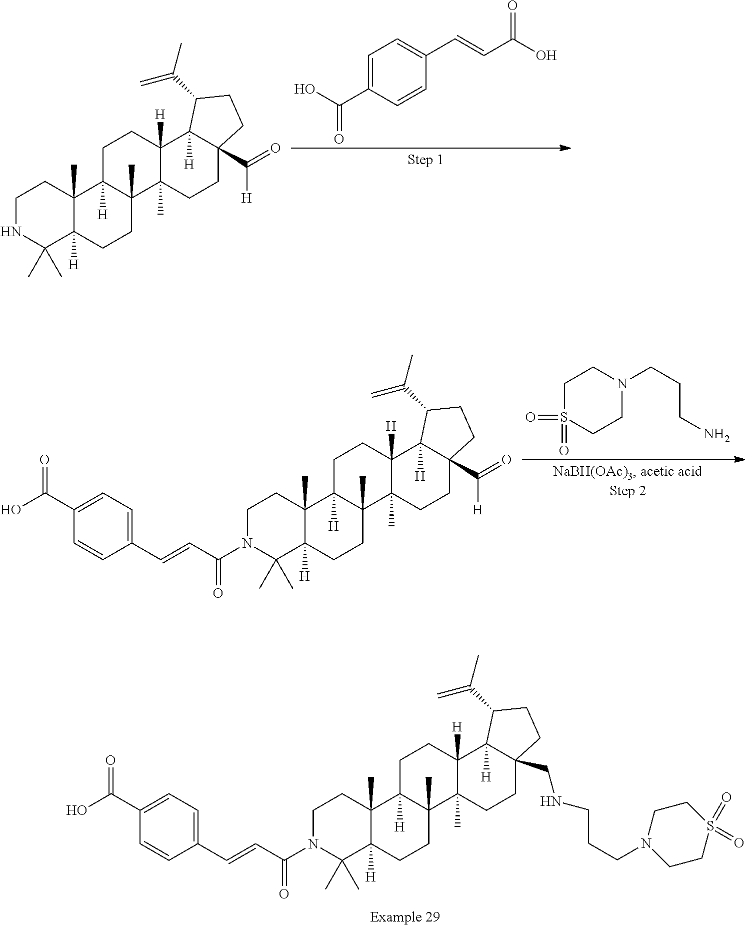

Step 1: Preparation of 4-((E)-3-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxoprop-1-en-1-yl)benzoic acid

The title compound was prepared in 71.0% yield following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoate, using (E)-4-(2-carboxyvinyl)benzoic acid instead of 4-tert-butoxy-3,3-dimethyl-4-oxobutanoic acid as the reactant. MS: m/e 600.37 (M+H)⁺, 1.85 min (method 4B).

Step 2: 4-((E)-3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxoprop-1-en-1-yl)benzoic acid was prepared in 20.9% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2,2-dimethyl-4-oxobutanoic acid, step 3, using 4-((E)-3-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-3-oxoprop-1-en-1-yl)benzoic acid instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 776.69 (M+H)⁺, 2.79 min (method 6B). ¹H NMR (400 MHz, METHANOL-d₄) δ 8.05 (d, J=8.5 Hz, 2H), 7.70 (d, J=8.3 Hz, 2H), 7.42 (d, J=15.6 Hz, 1H), 7.07 (d, J=15.6 Hz, 1H), 4.78 (s, 1H), 4.68 (s, 1H), 3.78-3.64 (m, 1H), 3.58-3.43 (m, 1H), 3.29-3.16 (m, 11H), 2.92-2.82 (m, 3H), 2.61-2.47 (m, 1H), 2.15-1.96 (m, 3H), 1.75 (s, 3H), 1.59 (s, 3H), 1.48 (s, 3H), 1.17 (s, 3H), 1.10 (s, 3H), 1.09 (s, 3H),1.95-1.07 (m, 21H).

### Example 30

### Preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-1H-cyclopenta[5,6]naphtho[2,1-f]isoquinoline-3-carbonyl)benzoic acid

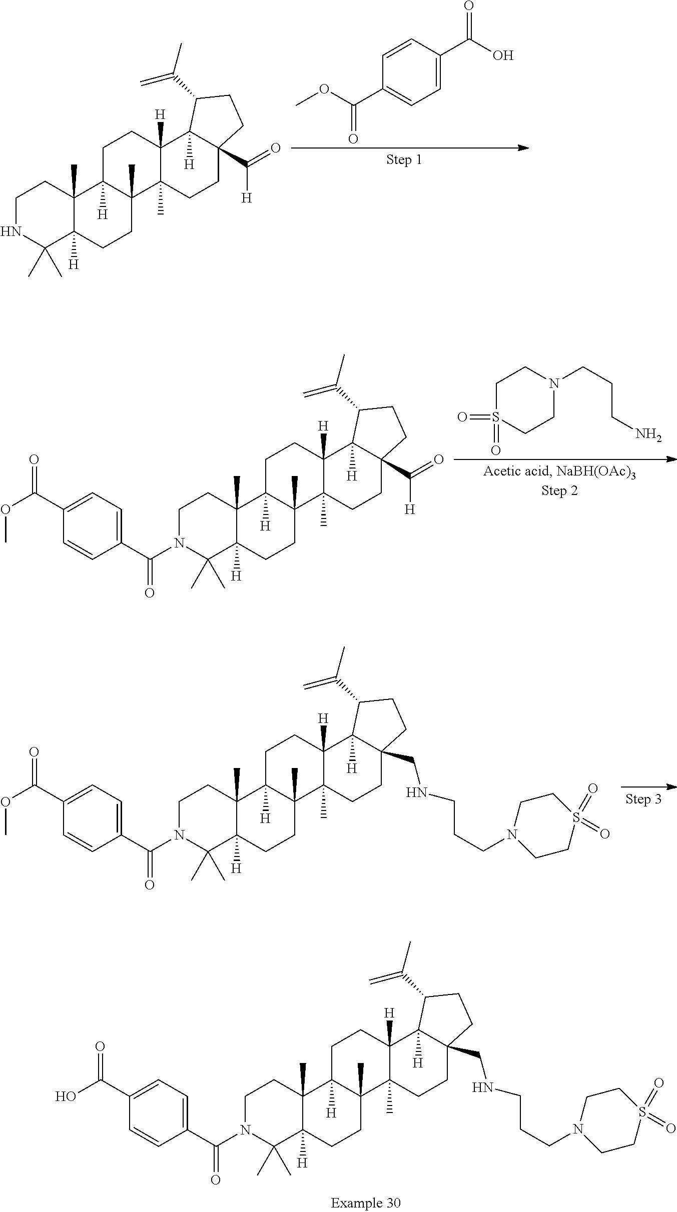

Step 1: Preparation of methyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-1H-cyclopenta[5,6]naphtho[2,1-f]isoquinoline-3-carbonyl)benzoate

The title compound was prepared in 72.4% yield following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoate, using 4-(methoxycarbonyl)benzoic acid instead of 4-tert-butoxy-3,3-dimethyl-4-oxobutanoic acid as the reactant. MS: m/e 588.38 (M+H)⁺, 2.04 min (method 4B).

Step 2: Preparation of methyl 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-1H-cyclopenta[5,6]naphtho[2,1-f]isoquinoline-3-carbonyl)benzoate.

The title compound was prepared in 27.8% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using methyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-1H-cyclopenta[5,6]naphtho[2,1-f]isoquinoline-3-carbonyl)benzoate instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 764.50 (M+H)⁺, 2.23 min (method 4B).

Step 3: 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-1H-cyclopenta[5,6]naphtho[2,1-f]isoquinoline-3-carbonyl)benzoic acid was prepared in 25.7% yield following the procedure described above for preparation 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid in step 3, using methyl 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-1H-cyclopenta[5,6]naphtho[2,1-f]isoquinoline-3-carbonyl)benzoate instead of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate as the reactant. MS: m/e 750.51 (M+H)⁺, 2.20 min (method 3B). ¹H NMR (400 MHz, methanol-d₄) δ 8.10 (d, J=8.3 Hz, 2H), 7.58-7.41 (m, 2H), 4.77 (s, 1H), 4.65 (s, 1H), 3.49-3.45 (m, 9H), 3.30-3.12 (m, 4H), 3.01-2.82 (m, 3H), 2.65-2.44 (m, 1H), 2.17-2.00 (m, 3H), 1.73 (s, 3H), 1.65 (s, 3H), 1.54 (s, 3H), 1.20 (s, 3H), 1.18 (s, 3H), 1.08 (s, 3H),1.89-1.03 (m, 21H).

### Example 31

### Preparation of 4-(((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)methyl)benzoic acid

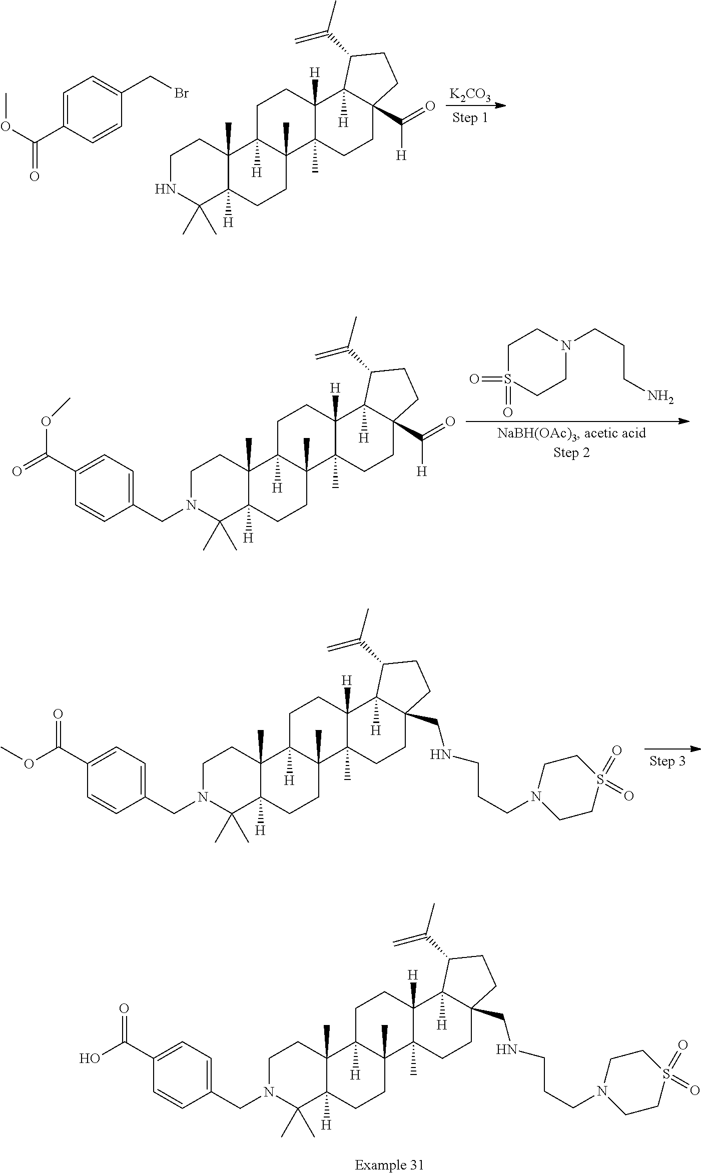

Step 1: Preparation of methyl 4-(((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)methyl)benzoate.

The title compound was prepared and used without purification following the procedure described above for (4aR,6aR,6bR,8aS,11R,11aR,11bR,13aR,13bR)-3-allyl-4,4,6a,6b,13b-pentamethyl-11-(prop-1-en-2-yl)icosahydro-1H-cyclopenta[5,6]naphtho[2,1-f]isoquinoline-8a-carbaldehyde, using methyl 4-(bromomethyl)benzoate instead of 3-bromoprop-1-ene as the reactant. MS: m/e 574.56 (M+H)⁺, 2.35 min (method 2B).

Step 2: Preparation of methyl 4-(((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)methyl)benzoate.

The title compound was prepared and used without purification following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2,2-dimethyl-4-oxobutanoic acid, step 3, using methyl 4-(((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)methyl)benzoate instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 750.45 (M+H)⁺, 1.86 min (method 3B).

Step 3: 4-(((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)methyl)benzoic acid was prepared in 16.38% yield following the procedure described above for preparation 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid in step 3, using methyl 4-(((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)methyl)benzoate instead of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate as the reactant. MS: m/e 736.61(M+H)⁺, 1.73 min (method 2B). ¹H NMR (400 MHz, methanol-d₄) δ 8.07-7.97 (m, 2H), 7.53-7.45 (m, 2H), 4.75 (s, 1H), 4.64 (s, 1H), 3.29-3.00 (m, 13H), 2.93 (m, 1H), 2.81 (d, J=12.8 Hz, 1H), 2.68 (t, J=6.5 Hz, 2H), 2.52 (d, J=6.8 Hz, 2H), 2.12-2.00 (m, 1H), 1.72 (s, 3H), 1.62 (s, 3H), 1.36 (s, 3H), 1.18 (s, 3H), 1.07 (s, 6H),1.86-1.03 (m, 23H).

### Example 32

### Preparation of (E)-4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-oxobut-2-enoic acid

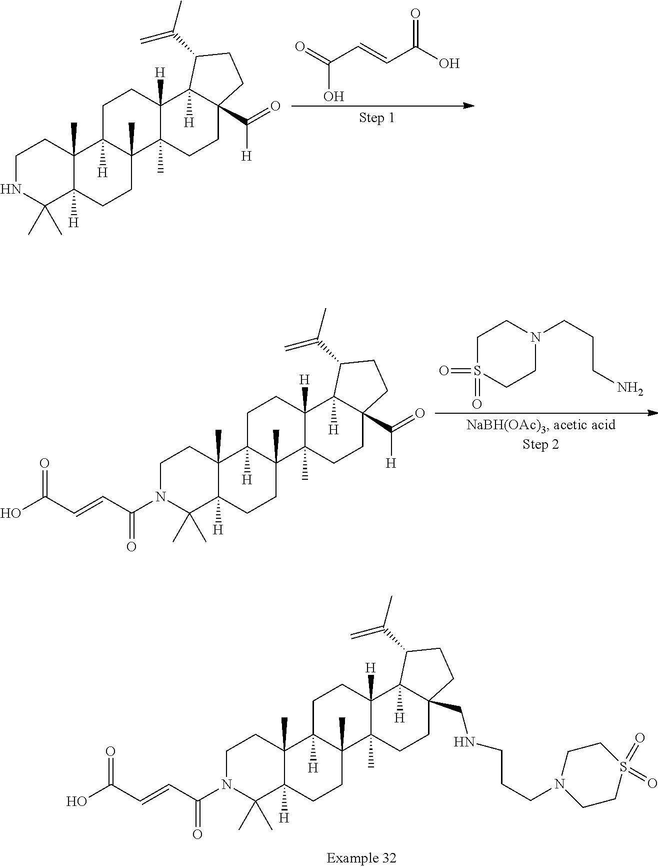

Step 1: Preparation of (E)-4-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-oxobut-2-enoic acid.

The title compound was prepared and used without purification following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahdro-3H-cyclopenta[5,6]naphtho[2,1-f]isoquinolin-3-yl)-2,2-dimethyl-4-oxobutanoate, using fumaric acid instead of 4-tert-butoxy-3,3-dimethyl-4-oxobutanoic acid as the reactant. MS: m/e 524.25 (M+H)⁺, 2.64 min (method 4B).

Step 2. (E)-4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-oxobut-2-enoic acid compound was prepared in 20.9% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using (E)-4-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-oxobut-2-enoic acid instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 700.48 (M+H)⁺, 2.12 min (method 3B). ¹H NMR (400 MHz, methanol-d₄) δ 7.00 (d, J=15.6 Hz, 1H), 6.56 (d, J=15.3 Hz, 1H), 4.77 (s, 1H), 4.67 (s, 1H), 3.62 (dd, J=14.6, 4.3 Hz, 1H), 3.51-3.37 (m, 1H), 3.27-3.09 (m, 7H), 3.04 (d, J=3.5 Hz, 4H), 2.83 (d, J=12.8 Hz, 1H), 2.68 (t, J=6.5 Hz, 2H), 2.53 (d, J=5.5 Hz, 1H), 2.16-2.00 (m, 1H), 1.74 (s, 3H), 1.56 (s, 3H), 1.43 (s, 3H), 1.15 (s, 3H), 1.08 (s, 3H), 1.06 (s, 3H),1.98-1.02 (m, 23H).

### Example 33

### Preparation of 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetic acid

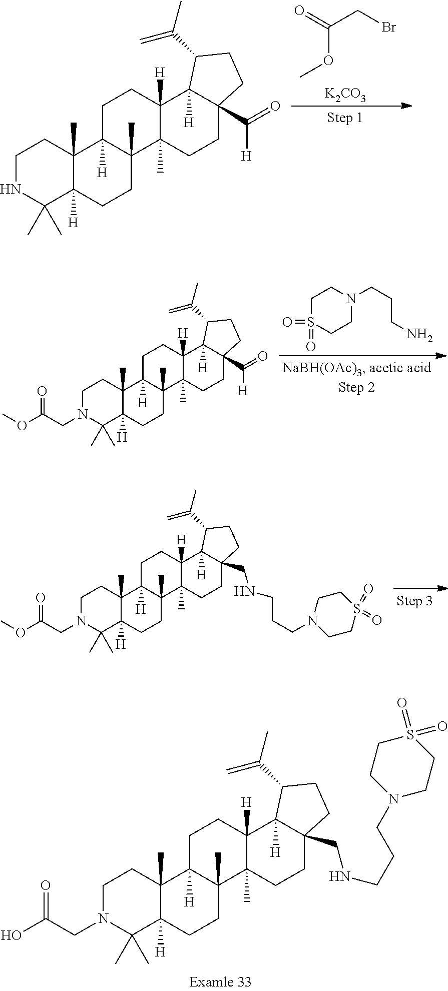

Step 1: Preparation of methyl 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetate

The title compound was prepared and without purification following the procedure described above for (4aR,6aR,6bR,8aS,11R,11aR,11bR,13aR,13bR)-3-allyl-4,4,6a,6b,13b-pentamethyl-11-(prop-1-en-2-yl)icosahydro-1H-cyclopenta[5,6]naphtho[2,1-f]isoquinoline-8a-carbaldehyde, using methyl 2-bromoacetate instead of 3-bromoprop-1-ene as the reactant. MS: m/e 498.29 (M+H)⁺, 2.07 min (method 4B).

Step 2: Preparation of methyl 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetate.

The title compound was prepared in a 42.1% yield by following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2,2-dimethyl-4-oxobutanoic acid, step 3, using methyl 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetate instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 674.55 (M+H)⁺, 1.68 min (method 2B). ¹H NMR (400 MHz, chloroform-d) δ 4.70 (s, 1H), 4.61 (s, 1H), 3.73 (s, 3H), 3.51 (br. s., 2H), 3.13-2.98 (m, 9H), 2.93-2.51 (m, 7H), 2.49-2.37 (m, 1H), 1.71 (s, 3H), 1.12 (s, 3H), 1.05 (s, 3H), 0.96 (s, 3H), 0.88 (s, 3H), 0.84 (s, 3H), 2.02-0.77 (m, 24H).

Step 3. 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetic acid was prepared in 44.5% yield following the procedure described above for preparation 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid in step 3, using 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetate instead of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate as the reactant. MS: m/e 660.46 (M+H)⁺, 1.45 min (method 4B). ¹H NMR (400 MHz, chloroform-d) δ 4.66 (s, 1H), 4.59 (s, 1H), 3.73 (d, J=12.8 Hz, 1H), 3.34 (d, J=1.8 Hz, 2H), 3.28 (d, J=13.3 Hz, 1H), 3.18-2.85 (m, 11H), 2.57 (t, J=5.6 Hz, 2H), 2.50 (d, J=12.8 Hz, 1H), 2.36 (br. s., 1H), 1.65 (s, 3H), 1.37 (s, 3H), 1.21 (s, 3H), 1.04 (s, 3H), 0.96 (s, 3H), 0.95 (s, 3H), 1.88-0.92 (m, 24H).

### Example 34

### Preparation of (E)-ethyl 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)but-2-enoate

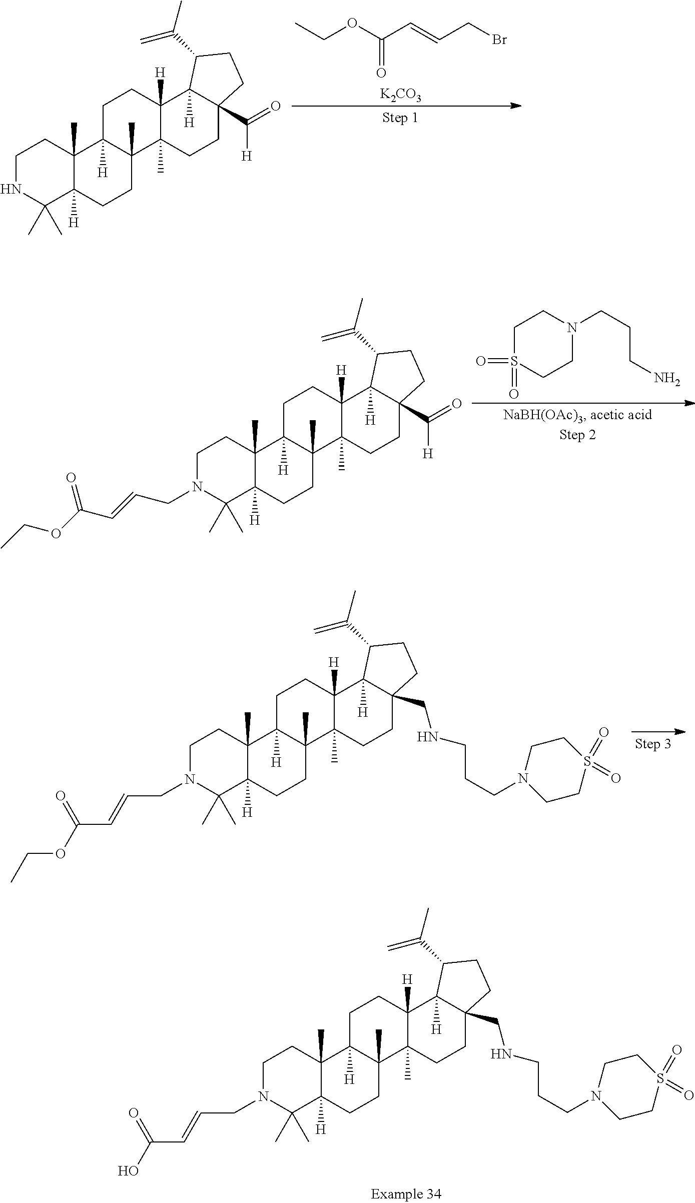

Step 1: Preparation of (E)-ethyl 4-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)but-2-enoate.

The title compound was prepared and used without purification following the procedure described above for (4aR,6aR,6bR,8aS,11R,11aR,11bR,13aR,13bR)-3-allyl-4,4,6a,6b,13b-pentamethyl-11-(prop-1-en-2-yl)icosahydro-1H-cyclopenta[5,6]naphtho[2,1-f]isoquinoline-8a-carbaldehyde, using (E)-ethyl 4-bromobut-2-enoate instead of 3-bromoprop-1-ene as the reactant. MS: m/e 538.34 (M+H)⁺, 2.12 min (method 4B).

Step 2: Preparation of (E)-ethyl 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)but-2-enoate.

The title compound was prepared in 24.5% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using (E)-ethyl 4-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)but-2-enoate instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. MS: m/e 714.63 (M+H)⁺, 1.88 min (method 2B). ¹H NMR (400 MHz, chloroform-d) δ 7.00 (dt, J=15.8, 5.4 Hz, 1H), 6.09-5.95 (m, 1H), 4.71 (s, 1H), 4.63 (s, 1H), 4.22 (quin, J=7.1 Hz, 2H), 3.44 (dd, J=5.4, 1.9 Hz, 1H), 3.18-2.95 (m, 14H), 2.79-2.36 (m, 4H), 2.07-0.81 (m, 45H).

Step 3. (E)-4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)but-2-enoic acid was prepared in 26.8% yield following the procedure described above for preparation 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid in step 3, using (E)-ethyl 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)but-2-enoate instead of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate as the reactant. MS: m/e 686.48(M+H)⁺, 1.45 min (method 4B). ¹H NMR (400 MHz, chloroform-d) δ 6.89-6.81 (m, 1H), 6.11 (d, J=15.3 Hz, 1H), 4.70 (s, 1H), 4.62 (s, 1H), 3.18-2.93 (m, 14H), 2.72-2.50 (m, 4H), 2.39 (br. s., 1H), 1.69 (s, 3H), 1.36 (s, 3H), 1.06 (s, 3H), 1.05 (s, 3H), 1.00 (s, 3H), 0.92 (s, 3H), 1.98-0.86 (m, 24H).

### Example 35

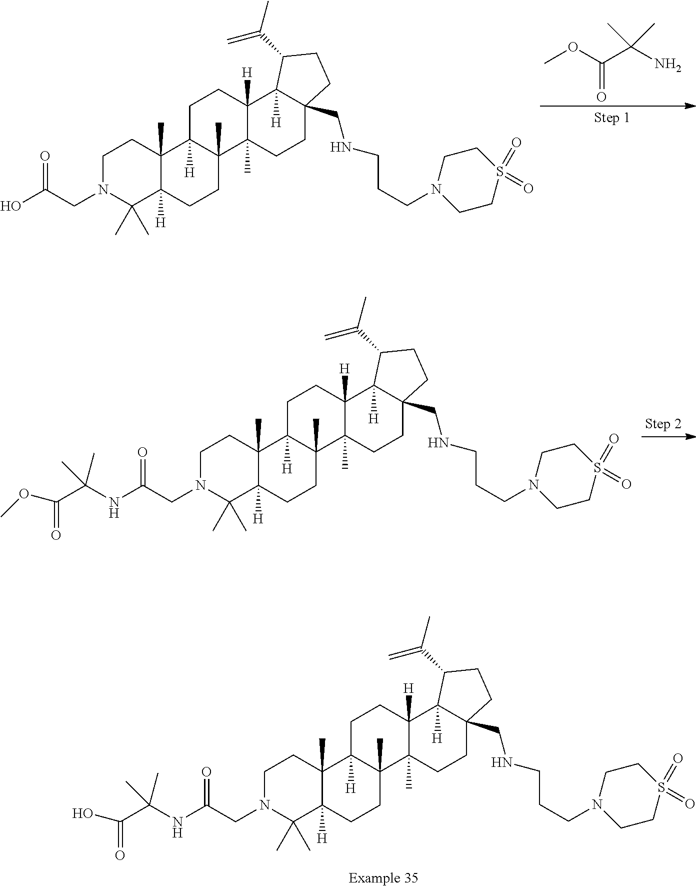

Step 1. Preparation of methyl 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetamido)-2-methylpropanoate

The title compound was prepared in 78% yield following the procedure described above for preparation of tert-butyl 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoate, using 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetic acid and methyl 2-amino-2-methylpropanoate instead of 4-tert-butoxy-3,3-dimethyl-4-oxobutanoic acid and (4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde as the reactants. MS: m/e 759.60 (M+H)⁺, 1.85 min (method 3B).

Step 2. 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetamido)-2-methylpropanoic acid was prepared in 36.7% yield following the procedure described above for preparation 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid in step 3, using methyl 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetamido)-2-methylpropanoate instead of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate as the reactant. LC/MS: m/e 745.57 (M+H)⁺, 1.73 min (method 3B). ¹H NMR (400 MHz, methanol-d₄) δ 4.77 (s, 1H), 4.67 (s, 1H), 3.45 (d, J=17.1 Hz, 1H), 3.21 (d, J=12.8 Hz, 1H), 3.19-3.10 (m, 5H), 3.05 (d, J=5.8 Hz, 4H), 2.84 (d, J=12.8 Hz, 1H), 2.78-2.72 (m, 1H), 2.68 (t, J=6.5 Hz, 3H), 2.61-2.48 (m, 3H), 2.16-2.01 (m, 2H), 1.98-1.81 (m, 5H), 1.75 (s, 3H), 1.79-1.71 (m, 3H), 1.55 (s, 3H), 1.54 (s, 3H), 1.63-1.42 (m, 8H), 1.41-1.20 (m, 4H), 1.16 (s, 3H), 1.12 (s, 6H), 1.20-1.10 (m, 4H), 0.97 (s, 3H), 0.93 (s, 3H).

### Example 36

### Preparation of 2-(2-((4aR,6aR,6bR,8aS,11R,11aR,11bR,13aR,13bR)-8a-(((3-(1,1-dioxidothiomorpholino)propyl)(methyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetamido)-2-methylpropanoic acid

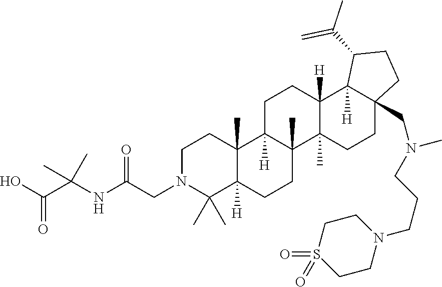

The title compound was prepared in 19.63% yield following the procedure described above for preparation 24-(3-((((4aR,6aR,6bR,8aS,11R,11aR,11bR,13aR,13bR)-3,4,4,6a,6b,13b-hexamethyl-11-(prop-1-en-2-yl)icosahydro-8aH-cyclopenta[5,6]naphtho[2,1-f]isoquinolin-8a-yl)methyl)(methyl)amino)propyl)thiomorpholine 1,1-dioxide, using 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)acetamido)-2-methylpropanoic acid instead of 4-(3-((((4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinolin-8a-yl)methyl)amino)propyl)thiomorpholine 1,1-dioxide as the reactant. LC/MS: m/e 759.60 (M+H)⁺, 1.73 min (method 2B). ¹H NMR (400 MHz, methanol-d₄) δ 4.76 (s, 1H), 4.65 (s, 1H), 3.59 (d, J=16.8 Hz, 1H), 3.18-3.08 (m, 5H), 3.04 (m, 6H), 2.92-2.79 (m, 1H), 2.76 (s, 3H), 2.79-2.69 (m, 2H), 2.65 (t, J=6.7 Hz, 2H), 2.51 (br. s., 1H), 2.11-1.99 (m, 1H), 1.97 (s, 3H), 1.95-1.83 (m, 4H), 1.74 (s, 3H), 1.82-1.67 (m, 4H), 1.60 (m, 1H), 1.55 (s, 3H), 1.54 (s, 3H), 1.57-1.41 (m, 8H), 1.38-1.21 (m, 4H), 1.19-1.13 (m, 2H), 1.18 (s, 3H), 1.15 (s, 3H), 1.10 (s, 3H), 0.99 (s, 3H), 0.98 (s, 3H).

### Example 37

### Preparation of 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetic acid

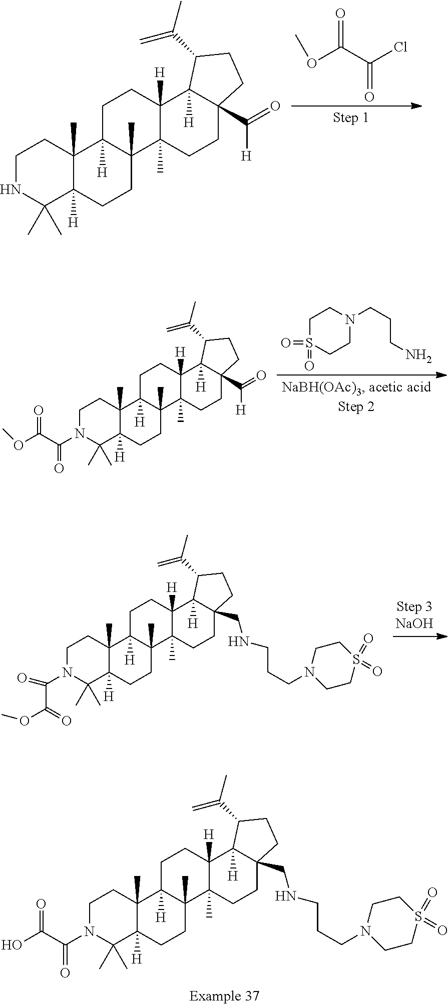

Step 1: Preparation of methyl 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetate

To a solution of (4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde (50 mg, 0.117 mmol) in dichloromethane (2 mL) at 0° C., was added methyl 2-chloro-2-oxoacetate (0.021 mL, 0.235 mmol) followed by DIEA (0.062 mL, 0.352 mmol). The resulting solution was stirred for 50 min and then diluted with 20 ml of ethyl acetate, washed with H₂O (2×10 ml) followed by brine (10 ml). The organic layer was dried over Na₂SO₄, filtered and concentrated under reduced pressure.

The resulting solid was used without further purification. LC/MS: m/e 512.43 (M+H)⁺, 2.72 min (method 2B).

Step 2: Preparation of methyl 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetate

The title compound was prepared in 55.8% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2,2-dimethyl-4-oxobutanoic acid, step 3, using methyl 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetate instead of 4-((4aR,6aR,6bR,8aS,11R,11aR,11bR,13aR,13bR)-8a-formyl-4,4,6a,6b,13b-pentamethyl-11-(prop-1-en-2-yl)icosahdro-3H-cyclopenta[5,6]naphtho[2,1-f]isoquinolin-3-yl)-2,2-dimethyl-4-oxobutanoic acid as the reactant. LC/MS: m/e 688.47 (M+H)⁺, 2.23 min (method 2B). ¹H NMR (400 MHz, chloroform-d) δ 4.70 (d, J=2.0 Hz, 1H), 4.61 (d, J=1.3 Hz, 1H), 3.25 (br. s., 2H), 3.04 (q, J=6.5 Hz, 10H), 2.82-2.66 (m, 2H), 2.61 (t, J=7.0 Hz, 2H), 2.43 (dt, J=11.0, 5.5 Hz, 1H), 2.26 (d, J=11.5 Hz, 1H), 2.01-1.85 (m, 2H), 1.85-1.65 (m, 6H), 1.70 (s, 3H), 1.65-1.50 (m, 3H), 1.53 (s, 3H), 1.47 (s, 3H), 1.51-1.30 (m, 8H), 1.31-1.18 (m, 4H), 1.07 (s, 3H), 1.13-1.06 (m, 2H), 1.02 (s, 3H), 1.06-1.00 (m, 1H), 0.99 (s, 3H).

Step 3. 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetic acid was prepared in 93% yield following the procedure described above for preparation 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetic acid in step 3, using methyl 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetate instead of methyl 2-(1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoethyl)cyclopentyl)acetate as the reactant. LC/MS: m/e 674.57 (M+H)⁺, 2.10 min (method 2B). ¹H NMR (400 MHz, methanol-d₄) δ 4.77 (s, 1H), 4.66 (s, 1H), 3.51 (br. s., 4H), 3.46-3.35 (br. s., 6H), 3.30-3.15 (m, 3H), 3.09 (br. s., 2H), 2.88 (d, J=13.1 Hz, 1H), 2.52 (d, J=5.5 Hz, 1H), 2.23-2.01 (m, 3H), 1.92-1.69 (m, 7H), 1.74 (s, 3H), 1.69-1.43 (m, 8H), 1.56 (s, 3H), 1.47 (s, 3H), 1.44-1.27 (m, 4H), 1.27-1.11 (m, 2H), 1.15 (s, 3H), 1.08 (s, 3H), 1.07 (s, 3H).

### Example 38

### Preparation of 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)-2-methylpropanoic acid

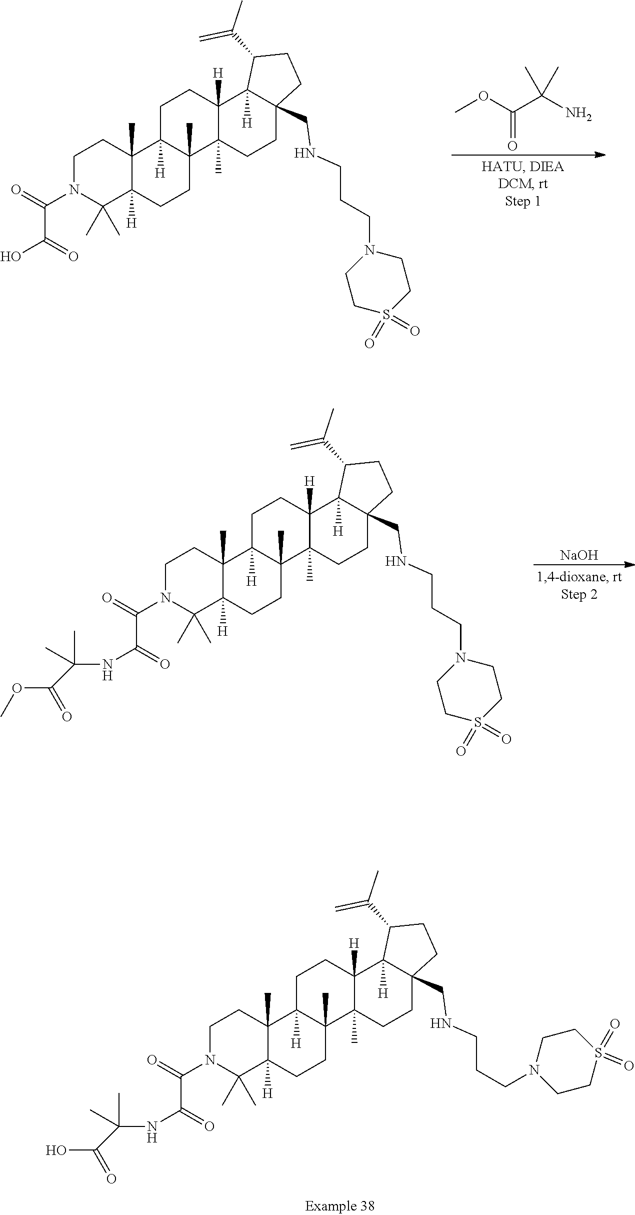

Step 1: Preparation of methyl 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)-2-methylpropanoate.

2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetic acid (50 mg, 0.074 mmol) was dissolved in DCM (2 mL) at 0° C., methyl 2-amino-2-methylpropanoate (8.69 mg, 0.074 mmol) was added, followed by HATU (56.4 mg, 0.148 mmol) and DIEA (0.039 mL, 0.223 mmol). The solution was stirred for 3 hours and and aqueous work up followed. The crude material was purified by flash chromatography using a 0-5% methanol in dichloromethane gradient. The title product was isolated as a white foam (0.030 g, 0.039 mmol, 53%). LCMS: m/e 773.6 (M+H)⁺, 2.13 min (method 8B).

Step 2: Methyl 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)-2-methylpropanoate (60 mg, 0.078 mmol) was dissolved in 1,4-dioxane (1.5 mL) and 1N sodium hydroxide (0.5 ml, 0.500 mmol)) was added. The reaction was stirred for 24 hrs then was purified by prep HPLC. Fractions containing the product were combined and concentrated under reduced pressure to give 2-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)-2-methylpropanoic acid as a white solid (0.040 g, 0.053 mmol, 68%). LCMS: m/e 759.6 (M+H)⁺, 2.04 min (method 8B). ¹H NMR (400 MHz, methanol-d₄) δ 4.75 (d, J=1.3 Hz, 1H), 4.64 (s, 1H), 3.62-3.52 (m, 5H), 3.46-3.36 (m, 5H), 3.26-3.08 (m, 5H), 2.88 (d, J=12.8 Hz, 1H), 2.51 (td, J=10.5, 5.9 Hz, 1H), 2.20-2.02 (m, 3H), 1.72 (s, 3H), 1.54 (s, 3H), 1.51 (s, 3H), 1.50 (s, 3H), 1.45 (s, 3H), 1.13 (s, 3H), 1.06 (s, 6H), 1.92-1.01 (m, 21H).

### Example 39 Preparation of 1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)cyclopropanecarboxylic acid

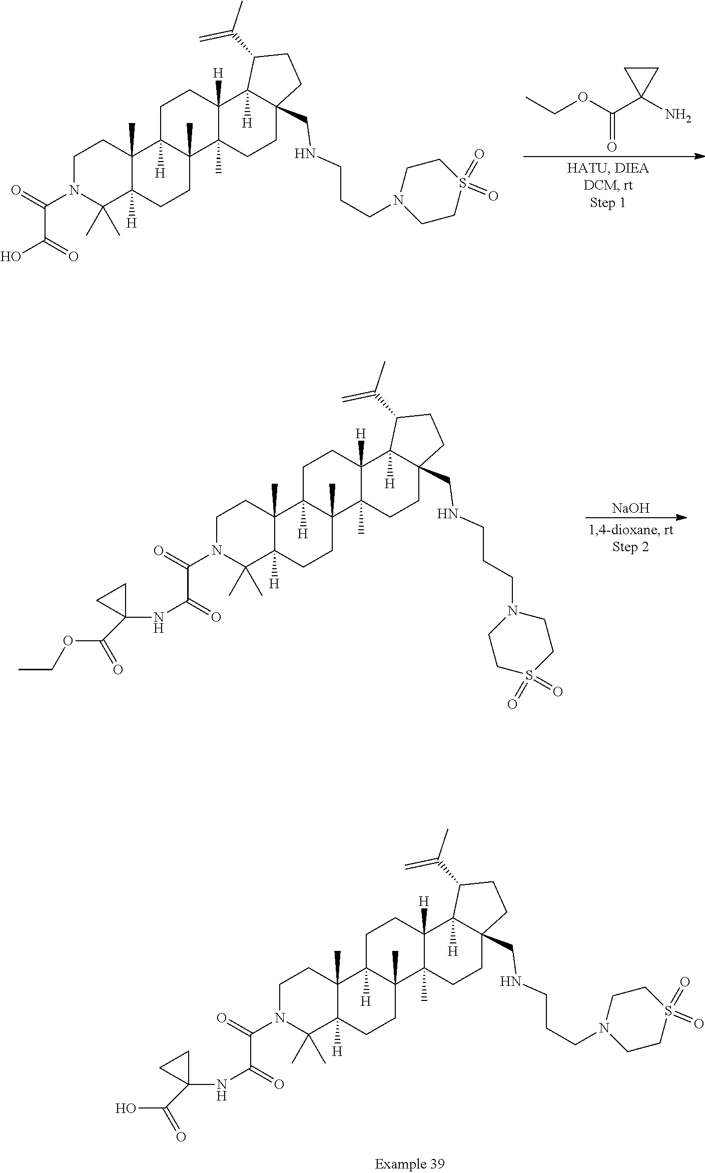

Step 1: Preparation of ethyl 1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)cyclopropanecarboxylate.

2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetic acid (40 mg, 0.059 mmol) in DCM (2 mL) was cooled to 0° C. and ethyl 1-aminocyclopropanecarboxylate (7.67 mg, 0.059 mmol) was added followed by HATU (45.1 mg, 0.119 mmol) and DIEA (0.031 mL, 0.178 mmol). The solution was stirred for 3 hours followed by an aqueous work up. The crude product was purified by flash chromatography using a 0-5% methanol in dichloromethane gradient. The title product was isolated as a white foam (15 mg, 0.019 mmol, 32%). LCMS: m/e 785.6 (M+H)⁺, 2.2 min (method 8B).

Step 2: Ethyl 1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)cyclopropanecarboxylate (40 mg, 0.051 mmol) was dissolved in 1,4-dioxane (1.5 mL) and 1N sodium hydroxide (0.5 ml, 0.500 mmol) was added. The reaction was stirred for 24 hrs at rt then was purified by prep HPLC. Fractions containing the product were combined and concentrated under reduced pressure to give 1-(2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-2-oxoacetamido)cyclopropanecarboxylic acid as a white solid (0.020 g, 0.026 mmol, 51%). LCMS: m/e 757.5 (M+H)⁺, 1.97 min (method 8B). ¹H NMR (400 MHz, methanol-d₄) δ 4.75 (d, J=1.3 Hz, 1H), 4.64 (s, 1H), 3.73-3.62 (m, 1H), 3.51-3.34 (m, 8H), 3.26-3.13 (m, 3H), 3.04 (t, J=7.2 Hz, 2H), 2.87 (d, J=13.1 Hz, 1H), 2.51 (td, J=10.4, 5.6 Hz, 1H), 2.17-2.01 (m, 3H), 1.72 (s, 3H), 1.54 (s, 3H), 1.45 (s, 3H), 1.17 (d, J=3.3 Hz, 2H), 1.14 (s, 3H), 1.06 (s, 6H), 1.91-1.04 (m, 24H).

### Example 40

### Preparation of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile

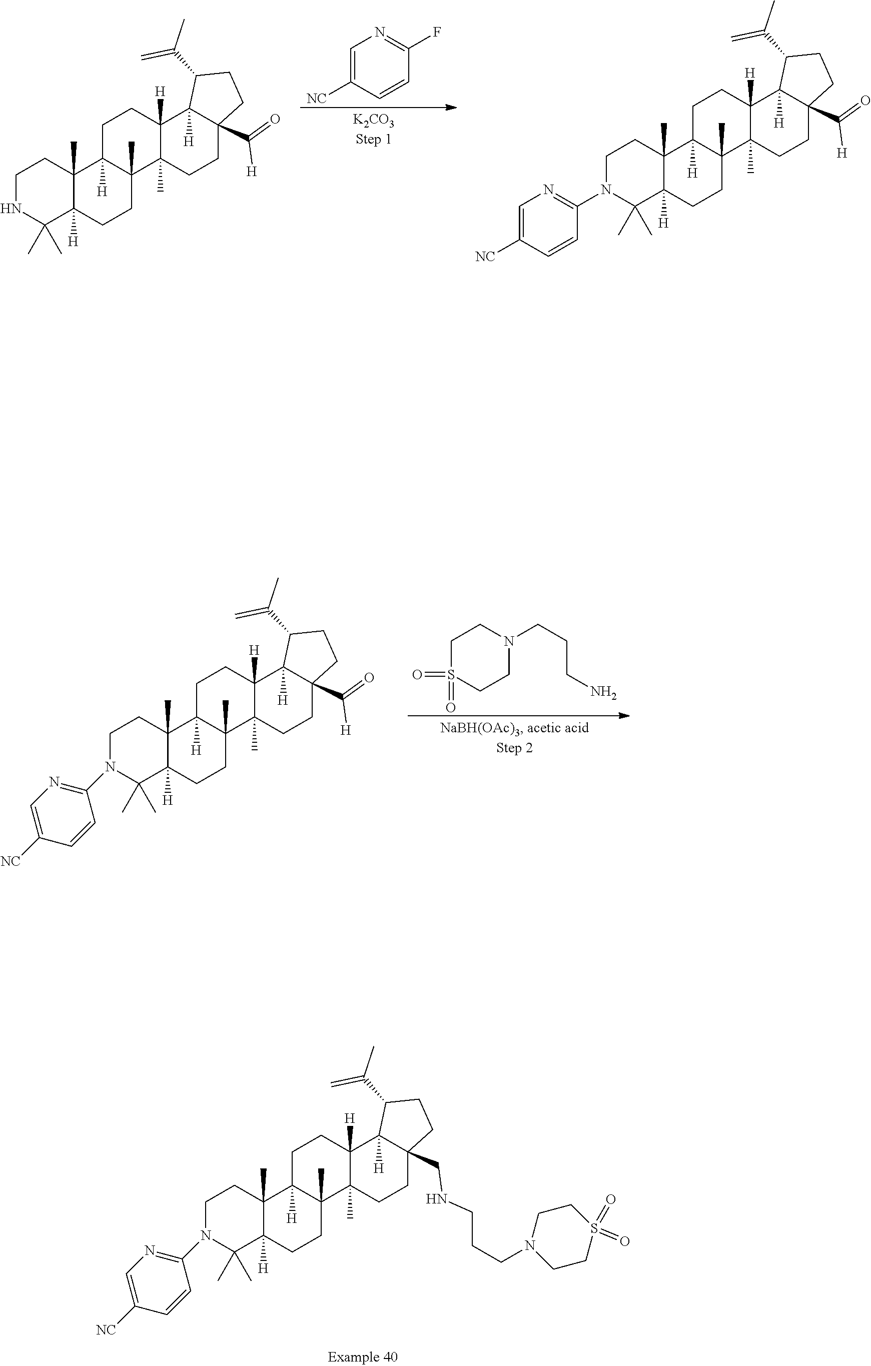

Step 1: Preparation of 6-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile To a mixture of (4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde (25.0 mg, 0.059 mmol), 6-fluoronicotinonitrile (71.7 mg, 0.587 mmol) and potassium carbonate (16.23 mg, 0.117 mmol) in a sealed tube was added 4 drops of DMSO. The tube was warmed to 130° C. for 3 hours. The reaction mixture was diluted with dichloromethane, washed with water and dried over sodium sulfate. The drying agent was removed by filtration and the filtrate was concentrated under reduced pressure. The crude mixture was purified by flash chromatography and the fractions containing product were collected and concentrated in vacuo to give the title compound as a white solid (29 mg, 94%). LC/MS: m/e 528.39 (M+H)⁺, 3.07 min (method 4B).

Step 2: 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile was prepared in 24.5% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using 6-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. LC/MS: m/e 704.50 (M+H)⁺, 2.45 min (method 2B). ¹H NMR (500 MHz, chloroform-d) δ 8.40 (dd, J=2.4, 0.6 Hz, 1H), 7.53 (dd, J=9.1, 2.5 Hz, 1H), 6.67 (d, J=8.8 Hz, 1H), 4.71 (d, J=2.0 Hz, 1H), 4.62 (s, 1H), 3.71-3.59 (m, 1H), 3.59-3.44 (m, 1H), 3.04-3.06 (m, 8H), 2.88-2.69 (m, 3H), 2.62 (t, J=6.9 Hz, 2H), 2.45 (td, J=11.1, 5.6 Hz, 1H), 2.28 (d, J=11.0 Hz, 1H), 2.04-1.87 (m, 2H), 1.87-1.73 (m, 4H), 1.71 (s, 3H), 1.70-1.53 (m, 5H), 1.51 (s, 3H), 1.49 (s, 3H), 1.47-1.39 (m, 6H), 1.39-1.20 (m, 5H), 1.09 (s, 3H), 1.15-1.04 (m, 3H), 1.04-1.00 (m, 3H), 0.98 (s, 3H).

### Example 41 and Example 42

### Preparation of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinamide and 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinic acid

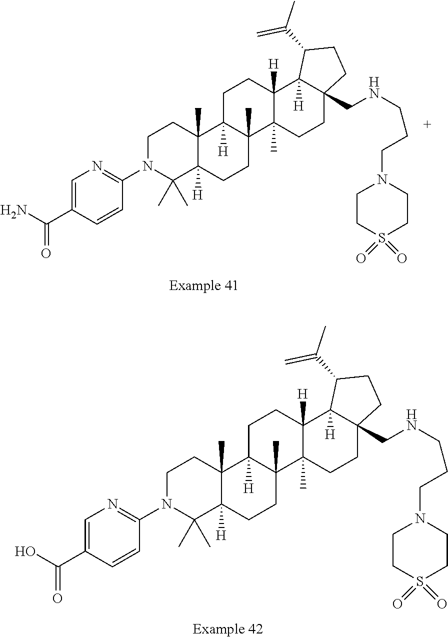

To a solution of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile (53 mg, 0.075 mmol) in ethanol (5 mL) was added a 40% w/w solution of potassium hydroxide (10 ml, 60 mmol). The mixture was warmed to 80° C. for 16 hours, then was cooled to rt and neutralized with 6N HCl to pH=4. The mixture was extracted twice with chloroform then the combined organic layers were washed with water and dried over sodium sulfate. The drying agent was removed by filtration and the filtrate was concentrated under reduced pressure. The crude material was purified by prep HPLC. The fractions containing each product were collected and concentrated in vacuo to give the two title compounds as white solids.

### Example 41

(5 mg, 8.74%). MS m/e 722.68 (M+H)⁺, 1.85 min (method 2B). ¹H NMR (400 MHz, methanol-d₄) δ 8.79 (d, J=2.3 Hz, 1H), 8.39 (dd, J=9.0, 2.3 Hz, 1H), 7.60 (d, J=8.8 Hz, 1H), 4.78 (s, 1H), 4.67 (s, 1H), 4.03 (m, 1H), 3.58 (m, 1H), 3.27 (br. s, 8H), 3.20 (d, J=9.8 Hz, 3H), 2.92-2.84 (m, 2H), 2.53 (br. s., 1H), 2.15-1.93 (m, 4H), 1.93-1.72 (m, 6H), 1.75 (s, 3H), 1.73-1.49 (m, 10H), 1.48 (s, 3H), 1.44 (s, 3H), 1.42-1.30 (m, 3H), 1.27-1.17 (m, 2H), 1.19 (s, 3H), 1.12 (s, 3H), 1.11 (s, 3H).

### Example 42

(45 mg, 79%). LC/MS: m/e 723.51 (M+H)⁺, 2.04 min (method 2B). ¹H NMR (400 MHz, methanol-d₄) δ 8.79 (d, J=2.0 Hz, 1H), 8.50-8.40 (m, 1H), 7.62 (d, J=9.3 Hz, 1H), 4.78 (d, J=1.5 Hz, 1H), 4.67 (s, 1H), 4.08-3.91 (m, 1H), 3.70-3.58 (m, 1H), 3.54 (dd, J=6.8, 3.5 Hz, 4H), 3.47-3.39 (m, 4H), 3.29-3.23 (m, 1H), 3.21 (dd, J=7.9, 3.1 Hz, 1H), 3.11 (t, J=7.3 Hz, 2H), 2.88 (d, J=13.1 Hz, 1H), 2.53 (td, J=10.5, 5.6 Hz, 1H), 2.22-2.12 (m, 2H), 2.12-2.05 (m, 1H), 2.05-1.94 (m, 1H), 1.94-1.86 (m, 1H), 1.86-1.69 (m, 6H), 1.75 (s, 3H), 1.60-1.70 (m, 6H), 1.58-1.44 (m, 4H), 1.50 (s, 3H), 1.47 (s, 3H), 1.42-1.28 (m, 2H), 1.27-1.15 (m, 2H), 1.18 (s, 3H), 1.11 (s, 3H), 1.09 (s, 3H).

### Example 43

### Preparation of 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinonitrile

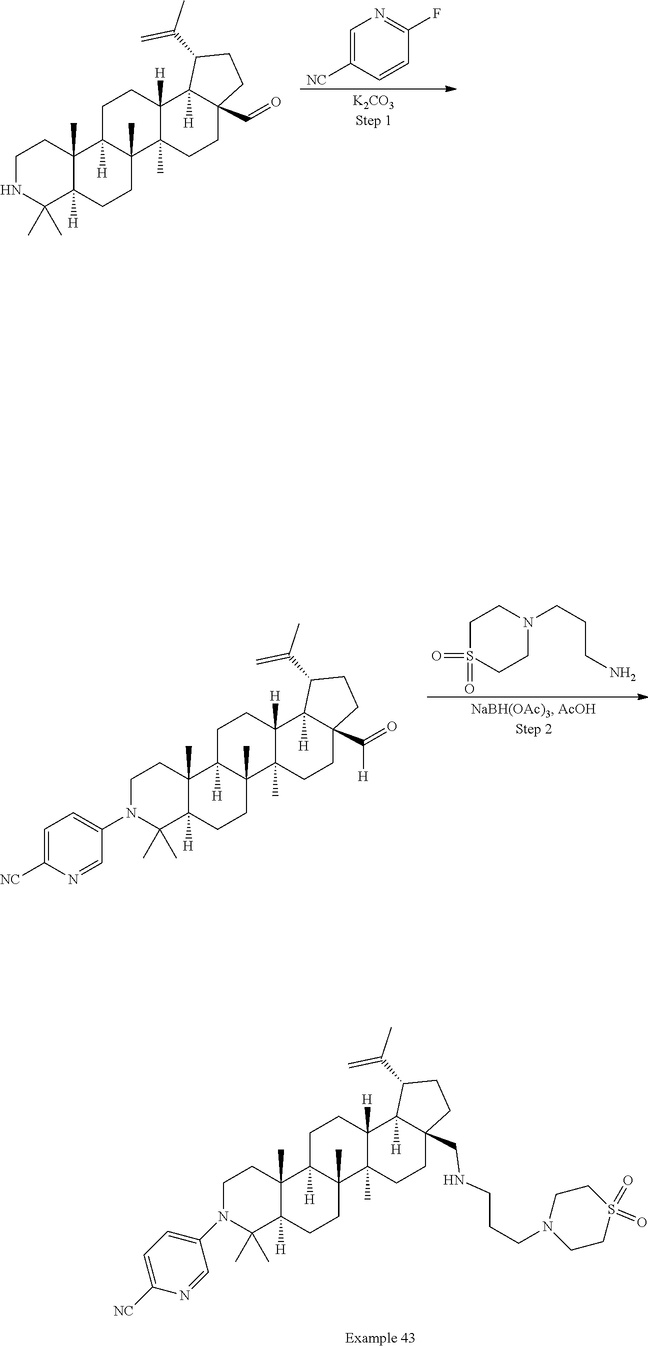

Step 1: Preparation of 5-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinonitrile.

The title compound was prepared in 29% yield following the procedure described above for preparation 6-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile, using 5-fluoropicolinonitrile instead of 6-fluoronicotinonitrile as the reactant. LC/MS: m/e 528.53 (M+H)⁺, 2.80 min (method 2B). ¹H NMR (400 MHz, chloroform-d) δ 9.69 (d, J=1.5 Hz, 1H), 8.45 (d, J=2.3 Hz, 1H), 7.62-7.55 (m, 1H), 7.47 (dd, J=8.5, 2.5 Hz, 1H), 4.79 (d, J=2.0 Hz, 1H), 4.69-4.61 (m, 1H), 3.57-3.45 (m, 1H), 3.01-2.85 (m, 2H), 2.15-2.02 (m, 2H), 1.98-1.85 (m, 1H), 1.72 (s, 3H), 1.62 (s, 3H), 1.14 (s, 3H), 1.03 (s, 3H), 1.01 (s, 3H), 0.97 (s, 3H), 1.83-0.94 (m, 19H).

Step 2: 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinonitrile was prepared in 59% yield following the procedure described above for preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid, step 3, using 5-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinonitrile instead of 4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)-2,2-dimethyl-4-oxobutanoic acid as the reactant. LC/MS: m/e 704.57 (M+H)⁺, 2.33 min (method 2B). ¹H NMR (400 MHz, Chloroform-d) δ 8.44 (d, J=2.3 Hz, 1H), 7.56 (d, J=8.3 Hz, 1H), 7.46 (dd, J=8.4, 2.6 Hz, 1H), 4.70 (d, J=1.8 Hz, 1H), 4.60 (s, 1H), 3.55-3.43 (m, 1H), 3.12-2.91 (m, 10H), 2.82-2.67 (m, 3H), 2.61 (t, J=6.9 Hz, 2H), 2.44 (td, J=11.1, 5.4 Hz, 1H), 2.26 (d, J=11.5 Hz, 1H), 1.98-1.85 (m, 2H), 1.85-1.63 (m, 7H), 1.69 (s, 3H), 1.60-1.36 (m, 8H), 1.36-1.17 (m, 4H), 1.14 (s, 3H), 1.08 (s, 3H), 1.18-0.97 (m, 2H), 1.01 (s, 6H), 1.00 (s, 3H).

### Example 44

### Preparation of 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinic acid

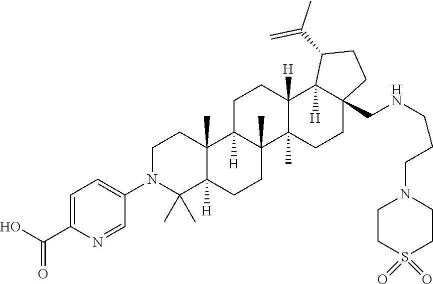

The title compound was prepared in 84% yield following the procedure described above for 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinic acid, using 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinonitrile instead of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)nicotinonitrile. LC/MS: m/e 723.53 (M+H)⁺, 1.78 min (method 4B). ¹H NMR (400 MHz, methanol-d₄) δ 8.78 (s, 1H), 8.28 (d, J=8.8 Hz, 1H), 8.25 (dd, J=2.01, 8.8 Hz, 1H), 4.75 (br. s, 1H), 4.63 (br. s, 1H), 4.12 (t, J=11.3 Hz, 1H), 3.58-3.48 (br. s, 4H), 3.45-3.33 (m, 5H), 3.27-3.14 (m, 3H), 3.10 (t, J=7.3 Hz, 2H), 2.85 (d, J=13.1 Hz, 1H), 2.52-2.48 (m, 1H), 2.21-1.98 (m, 3H), 1.96-1.69 (m, 7H), 1.72 (s, 3H), 1.69-1.39 (m, 5H), 1.30 (s, 3H), 1.27 (s, 3H), 1.40-1.24 (m, 8H), 1.21-1.02 (m, 2H), 1.16 (s, 3H), 1.13 (s, 3H), 1.08 (s, 3H).

### Example 45

### Preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2-fluorobenzoic acid

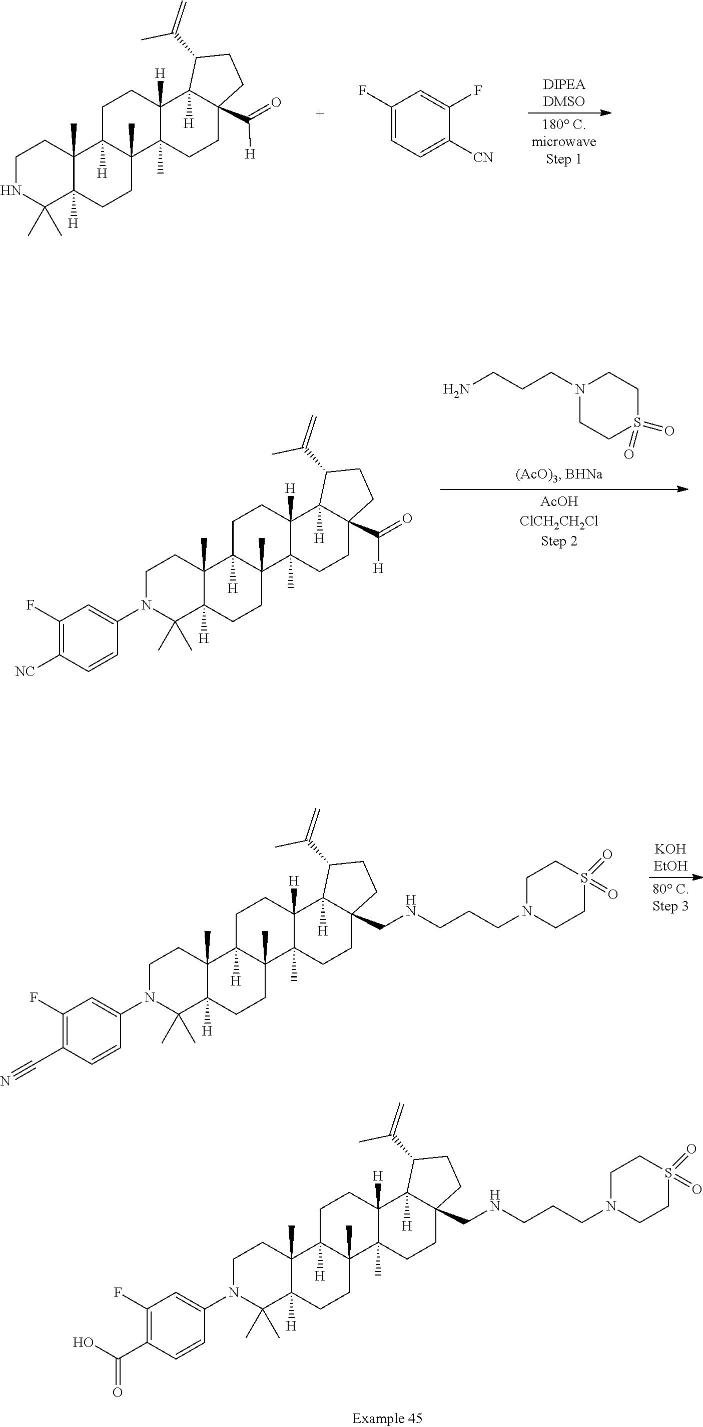

Step 1: Preparation of 2-fluoro-4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]-naphtho[2,1-f]isoquinolin-3-yl)benzonitrile

A mixture of (4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinoline-8a-carbaldehyde (30 mg, 0.07 mmol) 2,4-difluorobenzonitrile (196 mg, 1.40 mmol) and DIPEA (0.062 ml, 0.35 mmol) in DMSO (0.5 ml) was heated at 180° C. for 4 h. The reaction mixture was cooled to rt, diluted with H₂O (5 ml) and extracted with EtOAc (2×5 ml). The combined organic layers were washed with H₂O (5 ml), dried over Na₂SO₄, filtered and concentrated in vacuo to give the crude product which was used in next step without further purification. MS m/z 545.47 (M+H)⁺, 2.53 min (method 7B).

Step 2: Preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2-fluorobenzonitrile

A solution of 2-fluoro-4-((4aR,6aR,6bR,8aS,11R,11aR,11bR,13aR,13bR)-8a-formyl-4,4,6a,6b,13b-pentamethyl-11-(prop-1-en-2-yl)icosahydro-3H-cyclopenta[5,6]naphtho[2,1-f]isoquinolin-3-yl)benzonitrile (38 mg, 0.070 mmol), 4-(3-aminopropyl)thiomorpholine 1,1-dioxide (13 mg, 0.070 mmol) and AcOH (8 μL, 0.140 mmol) in DCE (2 ml) was stirred at rt for 30 min. Sodium triacetoxyborohydride (74 mg, 0.35 mmol) was added. The resulting mixture was stirred at rt overnight. LC/MS showed the reaction was incomplete. Additional 4-(3-aminopropyl)thiomorpholine 1,1-dioxide (13 mg, 0.070 mmol) and sodium triacetoxyborohydride (74 mg, 0.35 mmol) were added. The reaction was continued at rt for 3 days. The mixture was diluted with 5 ml of saturated NaHCO₃ and extracted with CH₂C₁₂ (3×10 ml). The combined organic layers were dried over Na₂SO₄, filtered and concentrated in vacuo. The crude product was purified by flash chromatography to give the title product as a solid. LC/MS m/z 721.64 (M+H)⁺, 1.86 min (method 7B).

Step 3: To a solution of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2-fluorobenzonitrile (10 mg, 0.014 mmol) in EtOH (1 ml) was added KOH (2 ml, 0.014 mmol). The resulting mixture was heated at 80° C. for 4 h, then was cooled to rt. The crude mixture was purified by Prep HPLC. Fractions containing the product were combined and concentrated under reduced pressure to give 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)icosahdro-3H-cyclopenta[5,6]naphtho[2,1-f]isoquinolin-3-yl)-2-fluorobenzoic acid as a solid in 17% yield. ¹H NMR (400 MHz, methanol-d₄) δ 8.08 (t, J=8.2 Hz, 1H), 7.55 (d, J=11.3 Hz, 1H), 7.48 (d, J=8.0 Hz, 1H), 4.76 (s, 1H), 4.65 (s, 1H), 4.18 (t, J=11.8 Hz, 1H), 3.22-3.11 (m, 7H), 3.09-3.03 (m, 4H), 2.86 (d, J=13.6 Hz, 1H), 2.70 (t, J=6.5 Hz, 2H), 2.57-2.47 (m, 1H), 2.10-1.20 (m, 27H), 1.73 (s, 3H), 1.28 (s, 6H), 1.18 (s, 3H), 1.16 (s, 3H), 1.10 (s, 3H). LC/MS m/z 740.67 (M+H)⁺, 1.28 min (method 7B).

### Example 46

### Preparation of 4-(3-((((4aR,6aR,6bR,8aS,11R,11aR,11bR,13aR,13bR)-4,4,6a,6b,13b-pentamethyl-3-(4-nitrophenyl)-11-(prop-1-en-2-yl)icosahydro-1H-cyclopenta[5,6]naphtho[2,1-f]isoquinolin-8a-yl)methyl)amino)propyl)thiomorpholine 1,1-dioxide

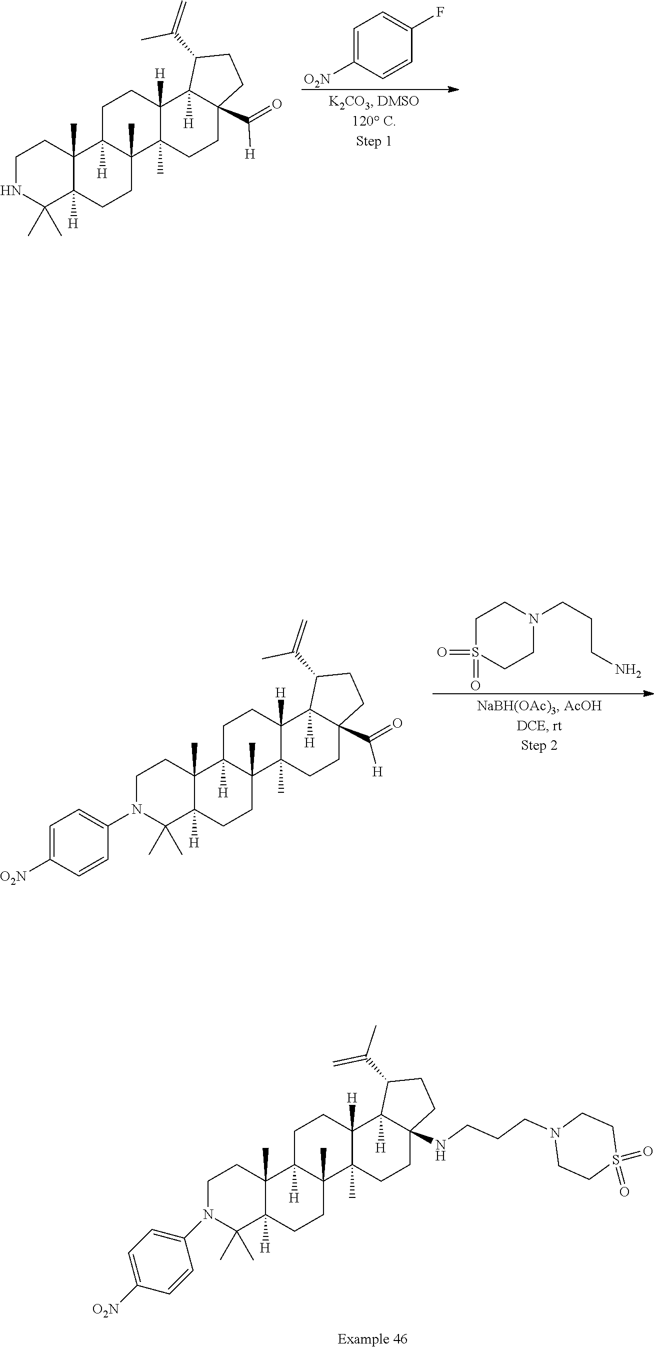

Step 1: Preparation of (4aR,6aR,6bR,8aS,11R,11aR,11bR,13aR,13bR)-4,4,6a,6b,13b-pentamethyl-3-(4-nitrophenyl)-11-(prop-1-en-2-yl)icosahydro-1H-cyclopenta[5,6]naphtho[2,1-f]isoquinoline-8a-carbaldehyde.

(4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde (150.0 mg, 0.352 mmol), 1-fluoro-4-nitrobenzene (300 mg, 2.126 mmol) and potassium carbonate (97 mg, 0.705 mmol) were added to a seal tube. The mixture was diluted with DMSO (0.6 mL) and the tube was sealed and heated 120° C. for 3 hours. The mixture was cooled to rt, diluted with dichloromethane and water. The organic layer was dried over sodium sulfate, filtered and concentrated under reduced pressure. The residue was purified by flash chromatography using a 0-5% ethyl acetate/Hexanes gradient. Fractions containing the product were combined and concentrated to give the title compound as a yellow solid (0.029 g, 0.053 mmol, 15%). LCMS: m/e 547.4 (M+H)⁺, 2.30 min (method 4B).

Step 2: To a solution of (4aR,6aR,6bR,8aS,11R,11aR,11bR,13aR,13bR)-4,4,6a,6b,13b-pentamethyl-3-(4-nitrophenyl)-11-(prop-1-en-2-yl)icosahydro-1H-cyclopenta[5,6]naphtho[2,1-f]isoquinoline-8a-carbaldehyde (40 mg, 0.073 mmol) in DCE (2 mL) was added acetic acid (8.37 μl, 0.146 mmol) and 4-(3-aminopropyl)thiomorpholine 1,1-dioxide (28.1 mg, 0.146 mmol). The mixture was stirred at rt for 2 h, then to the mixture was added sodium triacetoxyborohydride (78 mg, 0.366 mmol) and it was stirred overnight. The mixture was diluted with 7 mL of sat. sodium bicarbonate and was extracted with dichloromethane (3×7 mL). The combined organic layers were dried over sodium sulfate, filtered and concentrated under reduced pressure. The crude product was purified by flash chromatography using a 0%-5% methanol/methylene chloride gradient. The fractions containing the product were combined and concentrated under reduced pressure to give 4-(3-((((4aR,6aR,6bR,8aS,11R,11aR,11bR,13aR,13bR)-4,4,6a,6b,13b-pentamethyl-3-(4-nitrophenyl)-11-(prop-1-en-2-yl)icosahydro-1H-cyclopenta[5,6]naphtho[2,1-f]isoquinolin-8a-yl)methyl)amino)propyl)thiomorpholine 1,1-dioxide as a yellow foam (0.035 g, 0.048 mmol, 66%). LCMS: m/e 723.6 (M+H)⁺, 1.76 min (method 9B). ¹H NMR (400 MHz, chloroform-d) δ 8.11-8.06 (m, 2H), 7.16-7.08 (m, 2H), 4.70 (d, J=2.0 Hz, 1H), 4.60 (d, J=0.8 Hz, 1H), 3.10-2.98 (m, 9H), 2.79-2.66 (m, 3H), 2.61 (t, J=7.0 Hz, 2H), 2.43 (dt, J=11.0, 5.4 Hz, 1H), 2.24 (d, J=11.5 Hz, 1H), 1.70 (s, 3H), 1.19 (s, 3H), 1.07 (s, 3H), 1.06 (s, 3H), 1.01 (s, 3H), 1.00 (s, 3H), 2.02-0.94 (m, 26H).

### Example 47

### Preparation of 4-(3-((((4aR,6aR,6bR,8aS,11R,11aR,11bR,13aR,13bR)-3-(4-aminophenyl)-4,4,6a,6b,13b-pentamethyl-11-(prop-1-en-2-yl)icosahydro-1H-cyclopenta[5,6]naphtho[2,1-f]isoquinolin-8a-yl)methyl)amino)propyl)thiomorpholine 1,1-dioxide

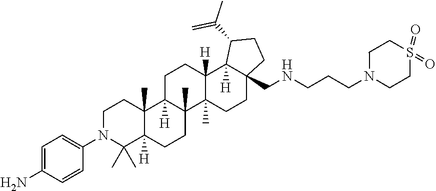

To a solution of 4-(3-((((4aR,6aR,6bR,8aS,11R,11aR,11bR,13aR,13bR)-4,4,6a,6b,13b-pentamethyl-3-(4-nitrophenyl)-11-(prop-1-en-2-yl)icosahydro-1H-cyclopenta[5,6]naphtho[2,1-f]isoquinolin-8a-yl)methyl)amino)propyl)thiomorpholine 1,1-dioxide (72 mg, 0.100 mmol) in abs. ethanol (9004) at was added tin(II) chloride dihydrate (112 mg, 0.498 mmol) in a single portion. The mixture was warmed to 70-72° C. for one hour. The mixture was diluted with ethyl acetate, washed by basic water and extracted with dichloromethane three times. The combined organic layers were dried over sodium sulfate, filtered and concentrated under reduced pressure. The residue was purified by flash chromatography using a 0-7.5% methanol/dichlormethane gradient. The fractions containing the product were combined and concentrated under reduced pressure to give the title product as an off-white solid (0.048 g, 0.069 mmol, 69%). LCMS: m/e 693.7 (M+H)⁺, 1.63 min (method 3B). ¹H NMR (400 MHz, CHLOROFORM-d) δ 6.99 (d, J=7.8 Hz, 2H), 6.59 (d, J=8.8 Hz, 2H), 4.69 (d, J=2.0 Hz, 1H), 4.59 (s, 1H), 3.55 (br. s., 2H), 3.48-3.38 (m, 1H), 3.05 (d, J=7.0 Hz, 8H), 2.73 (br. s., 4H), 2.61 (t, J=6.9 Hz, 2H), 2.43 (td, J=11.0, 5.6 Hz, 1H), 2.29-2.22 (m, 1H), 1.69 (s, 3H), 1.07 (s, 3H), 1.00 (s, 3H), 0.98 (s, 3H), 0.96 (br. s., 3H), 0.87 (s, 3H), 2.07-0.82 (m, 25H).

### Example 48

### Preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)benzoic acid

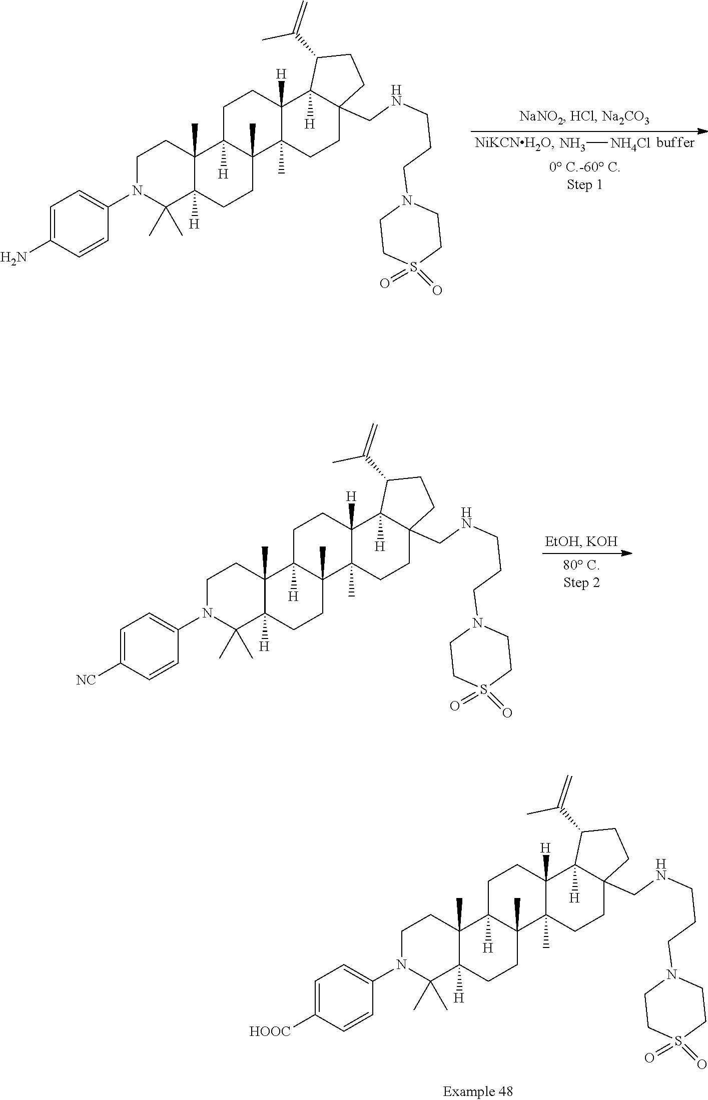

Step 1: Preparation of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)benzonitrile.

To a mixture of 4-(3-((((4aR,6aR,6bR,8aS,11R,11aR,11bR,13aR,13bR)-3-(4-aminophenyl)-4,4,6a,6b,13b-pentamethyl-11-(prop-1-en-2-yl)icosahydro-1H-cyclopenta[5,6]naphtho[2,1-f]isoquinolin-8a-yl)methyl)amino)propyl)thiomorpholine 1,1-dioxide (48 mg, 0.069 mmol) and 0.1M hydrochloric acid (1385 μl, 0.139 mmol) cooled to 0° C. was added a solution of sodium nitrite (5.73 mg, 0.083 mmol) in 1 ml of water dropwise. The mixture was stirred for 20 min at 0° C. then was neutralized with saturated aqueous sodium carbonate (14.68 mg, 0.139 mmol) at 0° C. The reaction mixture was added to a solution of nickel(II)potassium cyanide monohydrate (71.7 mg, 0.277 mmol) in 2 ml of NH₃—NH₄Cl buffer (pH=10) then was stirred at 50-60° C. for 30 min. The mixture was extracted with dichloromethane three times. The combined organic layers were dried over sodium sulfate, filtered and concentrated under reduced pressure. The residue was purified by flash chromatography using a 0-3% methanol/dichloromethane gradient. The fractions containing the product were combined and concentrated under reduced pressure to give the title product. LCMS: m/e 703.7 (M+H)⁺, 1.87 min (method 3B).

Step 2: To a solution of 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)benzonitrile (15 mg, 0.021 mmol) in ethanol (1 mL) was added a 40% w/w solution of potassium hydroxide (2 mL, 12.00 mmol). The mixture was warmed to 80° C. for 16 hours then was cooled to rt and purified by prep HPLC. The fractions containing the product were combined and concentrated under reduced pressure to give 4-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)benzoic acid t as a white solid (0.002 g, 0.003 mmol, 14%). LCMS: m/e 722.7 (M+H)⁺, 1.73 min (method 3B).

### Example 49 and Example 50

The preparation of 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carboxylic acid and 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carboxamide

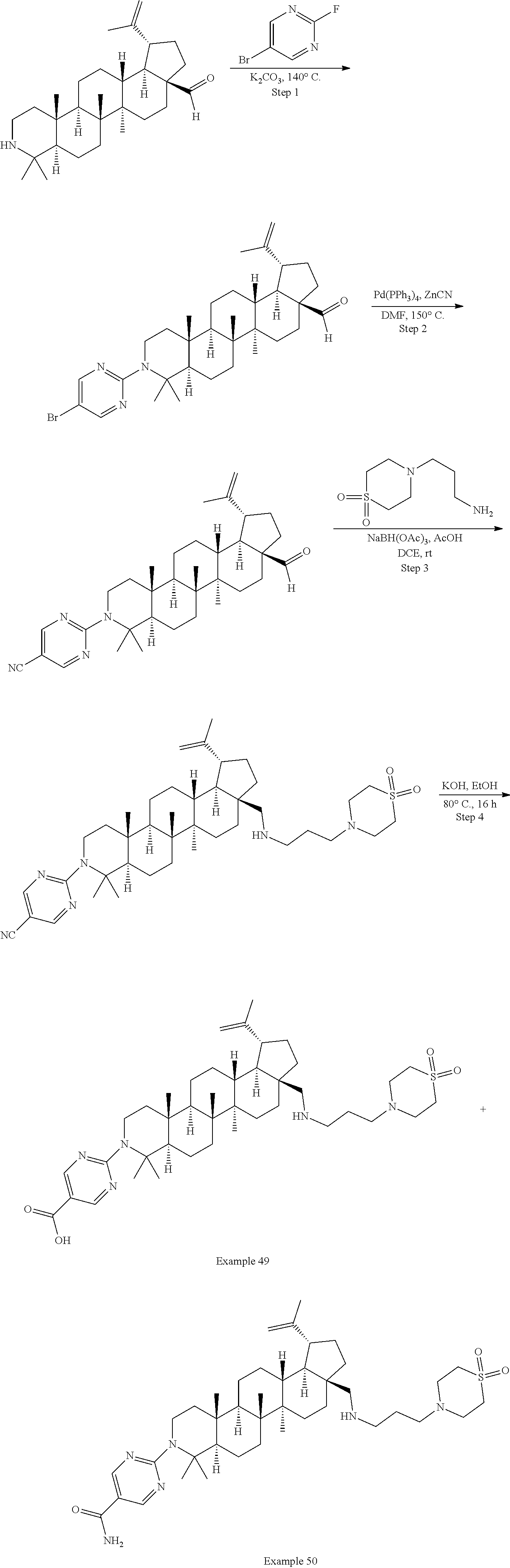

Step 1: Preparation of (4aR,6aR,6bR,8aS,11R,11aR,11bR,13aR,13bR)-3-(5-bromopyrimidin-2-yl)-4,4,6a,6b,13b-pentamethyl-11-(prop-1-en-2-yl)icosahydro-1H-cyclopenta[5,6]naphtho[2,1-f]isoquinoline-8a-carbaldehyde.

(4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde (90.0 mg, 0.211 mmol), 5-bromo-2-fluoropyrimidine (374 mg, 2.114 mmol) and potassium carbonate (58.4 mg, 0.423 mmol) were added to small pressure vessel. The vessel was sealed and warmed to 140° C. for 2 hours. The resulted mixture was diluted with methylene chloride, washed with water, dried over sodium sulfate, filtered and concentrated under reduced pressure. The crude material was purified by flash chromatography using a 0.1-3% ethyl acetate/hexanes gradient. After concentrating the fractions, the title product was isolated as a white solid (0.095 g, 0.163 mmol, 77%). LCMS: m/e 582.4 and 584.4 (M+H)⁺, 3.82 min (method 8B).

Step 2: Preparation of 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile

To a mixture of (4aR,6aR,6bR,8aS,11R,11aR,11bR,13aR,13bR)-3-(5-bromopyrimidin-2-yl)-4,4,6a,6b,13b-pentamethyl-11-(prop-1-en-2-yl)icosahydro-1H-cyclopenta[5,6]naphtho[2,1-f]isoquinoline-8a-carbaldehyde (95 mg, 0.163 mmol) and zinc cyanide (0.012 mL, 0.196 mmol) in DMF (2 mL) was added palladium tetrakis (18.84 mg, 0.016 mmol). The mixture was heated to 150° C. for 12 h, then was cooled to rt and washed with dichloromethane. The organic layer was washed with water and then the aqueous was extracted with dichloromethane three times. The combined organic layers were dried over sodium sulfate, filtered and concentrated under reduced pressure. The residue was purified by flash chromatography using a 0-3% ethyl acetate/hexanes gradient. The fractions containing the title product were combined and concentrated to give a white solid (0.031 g, 0.059 mmol, 36%). LCMS: m/e 529.45 (M+H)⁺, 3.33 min (method 4B).

Step 3: Preparation of 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile.

To a solution of 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile (10 mg, 0.019 mmol) in DCE (2 mL) was added acetic acid (2.163 0.038 mmol) and 4-(3-aminopropyl)thiomorpholine 1,1-dioxide (7.27 mg, 0.038 mmol). The mixture was stirred at rt for 2 h, then to the mixture was added sodium triacetoxyborohydride (20.04 mg, 0.095 mmol). The mixture was stirred at rt overnight then was diluted with 7 mL of saturated sodium bicarbonate and was extracted with dichloromethane (3×7 mL). The combined organic layers were dried over sodium sulfate, filtered and concentrated under reduced pressure. The crude product was purified by flash chromatography using a 0%-5% methanol/methylene chloride gradient. Fraction containing the product were combined and concentrated under reduced pressure to give the title compound as a white solid (10 mg, 0.014 mmol, 75%). LCMS: m/e 705.67 (M+H)⁺, 2.29 min (method 4B).

Step 4: To a solution of 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile (24 mg, 0.034 mmol) in ethanol (1 mL) was added a 40% w/w solution of KOH (2.0 mL, 12.0 mmol). The mixture was warmed to 80° C. for 16 hours then was cooled to rt and purified by prep HPLC. The fractions containing each isolate were concentrated under reduced pressure to provide:

### Example 49

2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carboxylic acid was isolated as a white solid (0.017 g, 0.023 mmol, 69%); LCMS: m/e 724.67 (M+H)⁺, 2.24 min (method 3B).

### Example 50

2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carboxamide was isolated as a film (0.003 g, 4.15 μmol, 12%); LCMS: m/e 723.66 (M+H)⁺, 2.13 min (method 3B).

### Example 51

### Preparation of 5-((4aR,6aR,6bR,8aS,11R,11aR,11bR,13aR,13bR)-8a-(((3-(1,1-dioxidothiomorpholino)propyl)(methyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinic acid

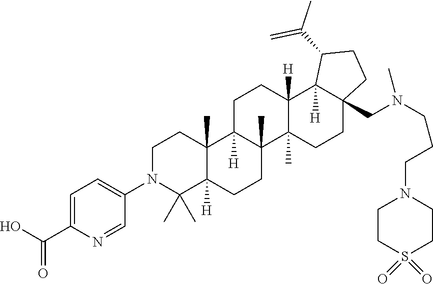

To a mixture of 5-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)picolinic acid (30 mg, 0.041 mmol) in MeOH (5.0 mL) was added formaldehyde (1.3 mg, 0.041 mmol) and acetic acid (2.4 4, 0.041 mmol). The mixture was stirred at rt for 30 minutes and sodium cyanoborohydride was added (2.6 mg, 0.041 mmol). The reaction was monitored by LC/MS. When product was observed the mixture was purified by prep HPLC to give the title product as a white foam. LCMS: m/e 737.7 (M+H)⁺, 1.84 min (method 3B).

### Example 52

### Preparation of (4aR,6aR,6bR,8aS,11R,11aR,11bR,13aR,13bR)-3-(5-carboxypyrimidin-2-yl)-4,4,6a,6b,13b-pentamethyl-11-(prop-1-en-2-yl)icosahydro-1H-cyclopenta[5,6]naphtho[2,1-f]isoquinoline-8a-carboxylic acid

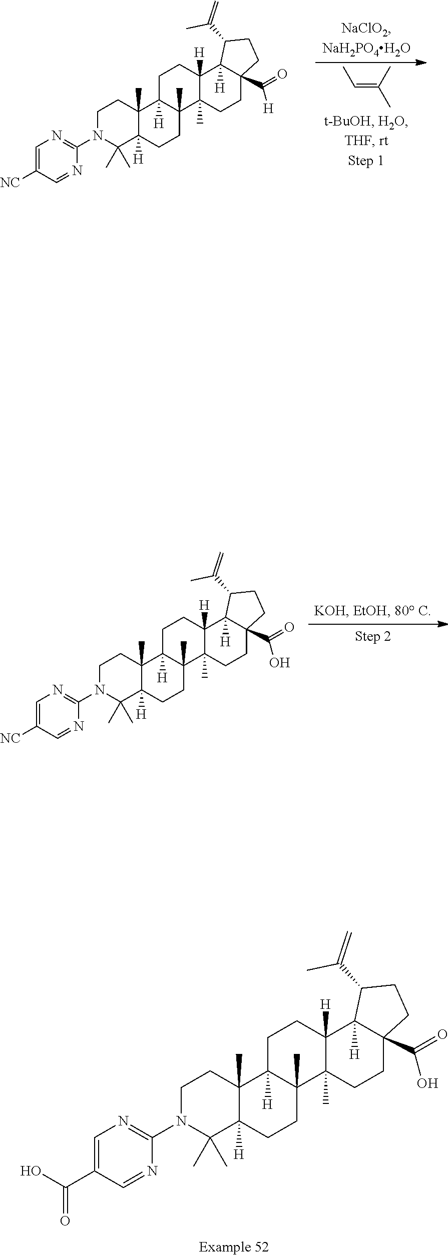

Step 1: Preparation of (4aR,6aR,6bR,8aS,11R,11aR,11bR,13aR,13bR)-3-(5-cyanopyrimidin-2-yl)-4,4,6a,6b,13b-pentamethyl-11-(prop-1-en-2-yl)icosahydro-1H-cyclopenta[5,6]naphtho[2,1-f]isoquinoline-8a-carboxylic acid.

To a solution of 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile (25 mg, 0.047 mmol) in t-BuOH (0.75 mL) was added 2-methyl-2-butene (0.922 mL, 1.844 mmol). A solution of sodium chlorite (34.2 mg, 0.378 mmol) and sodium phosphate monobasic monohydrate (67.9 mg, 0.492 mmol) in water (0.75 mL) was added dropwise over 10 min. The light-yellow solution was stirred at rt for 30 minutes at which point all starting material had been consumed (monitored by TLC). The mixture was diluted with water (50 mL) and was extracted with ethyl acetate (3×75 mL). The combined organic layers were washed with brine, dried over magnesium sulfate, filtered and concentrated under reduced pressure. The residue was purified by flash chromatography using a 0-5% methanol in dichloromethane gradient and a 4 g silica gel column. The fractions containing the product were combined concentrated under reduced pressure to give the title compound (22 mg, 0.040 mmol, 85% yield) as a white solid. LCMS: m/e 545.46 (M+H)⁺, 3.04 min (method 3B).

Step 2: To a solution of (4aR,6aR,6bR,8aS,11R,11aR,11bR,13aR,13bR)-3-(5-cyanopyrimidin-2-yl)-4,4,6a,6b,13b-pentamethyl-11-(prop-1-en-2-yl)icosahydro-1H-cyclopenta[5,6]naphtho[2,1-f]isoquinoline-8a-carboxylic acid (13 mg, 0.024 mmol) in ethanol (1 mL) was added a 40% w/w solution of potassium hydroxide (2.0 mL, 12.0 mmol). The mixture was warmed to 80° C. for 2 hours, then was cooled to rt and purified by prep HPLC. The fractions containing the product were combined and concentrated under reduced pressure to give (4aR,6aR,6bR,8aS,11R,11aR,11bR,13aR,13bR)-3-(5-carboxypyrimidin-2-yl)-4,4,6a,6b,13b-pentamethyl-11-(prop-1-en-2-yl)icosahydro-1H-cyclopenta[5,6]naphtho[2,1-f]isoquinoline-8a-carboxylic acid as a white solid (0.009 g, 0.016 mmol, 67%). LCMS: m/e 564.48 (M+H)⁺, 2.84 min (method 3B).

### Example 53

### Preparation of 2-((4aR,6aR,6bR,8aS,11R,11aR,11bR,13aR,13bR)-8a-((2-(1,1-dioxidothiomorpholino)ethyl)amino)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carboxylic acid

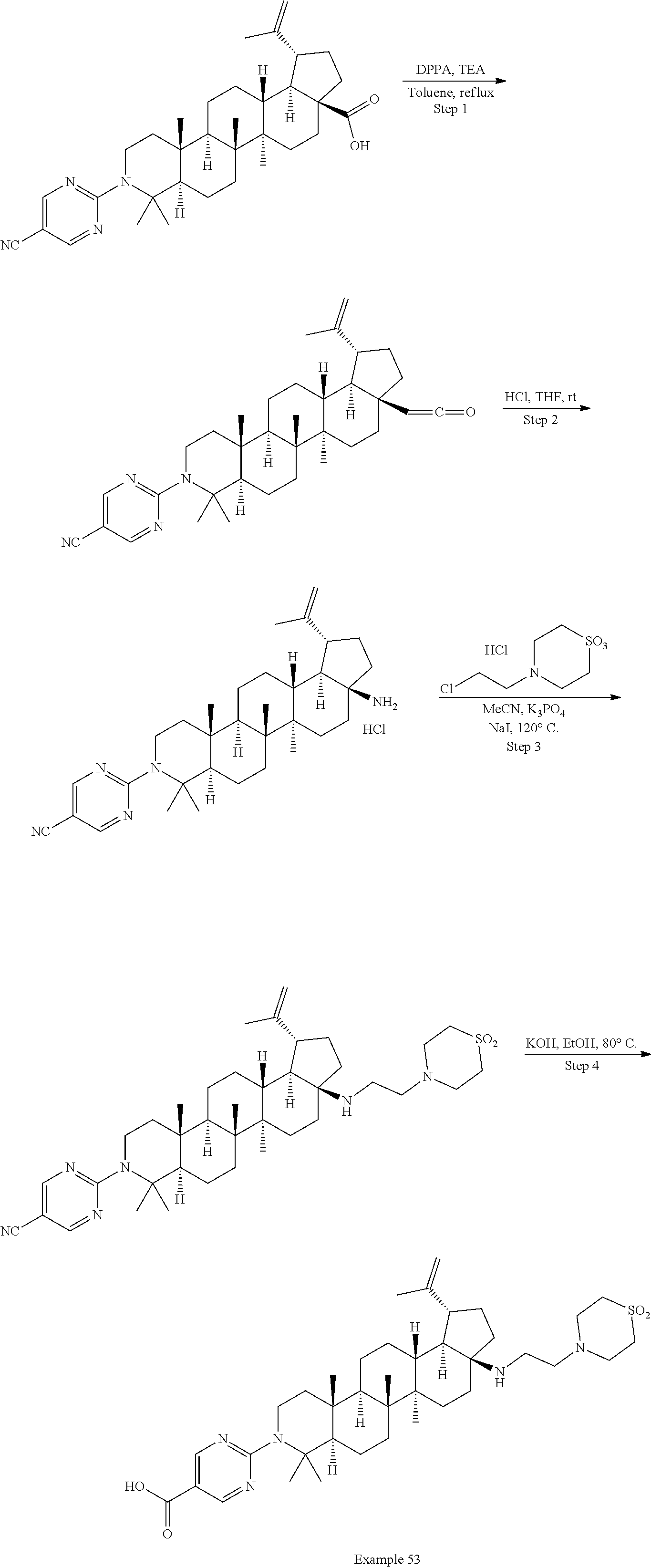

Step 1: Preparation of 2-((4aR,6aR,6bR,8aS,11R,11aR,13aR,13bR)-8a-isocyanato-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile.

To a solution of (4aR,6aR,6bR,8aS,11R,11aR,13aR,13bR)-3-(5-cyanopyrimidin-2-yl)-4,4,6a,6b,13b-pentamethyl-11-(prop-1-en-2-yl)icosahydro-1H-cyclopenta[5,6]naphtho[2,1-f]isoquinoline-8a-carboxylic acid (115 mg, 0.211 mmol) in toluene (8 mL) was added triethylamine (0.059 mL, 0.422 mmol) and diphenylphosphoryl azide (0.068 mL, 0.317 mmol). The mixture was heated to reflux for 1 h then was cooled to rt and concentrated under reduced pressure. The residue was purified by flash chromatography using a 5-10% ethyl acetate/hexanes gradient. The fractions containing the product were combined and concentrated under reduced pressure to give the title product as a white solid (0.090 g, 0.166 mmol, 79%). LCMS: m/e 542.48 (M+H)⁺, 4.12 min (method 4B).

Step 2: Preparation of 2-((4aR,6aR,6bR,8aS,11R,11aR,13aR,13bR)-8a-amino-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile hydrochloride.

To a solution of 2-((4aR,6aR,6bR,8aS,11R,11aR,13aR,13bR)-8a-isocyanato-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile (90 mg, 0.166 mmol) in THF (300 mL) was added HCl (37%, 41 mL, 496 mmol). The mixture was stirred at rt for 36 hrs. The solvent was removed to give product as off-white foam, which was directly used in the next step without further purification.

Step 3: Preparation of 2-((4aR,6aR,6bR,8aS,11R,11aR,11bR,13aR,13bR)-8a-((2-(1,1-dioxidothiomorpholino)ethyl)amino)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile.

A mixture of 2-((4aR,6aR,6bR,8aS,11R,11aR,11bR,13aR,13bR)-8a-amino-4,4,6a,6b,13b-pentamethyl-11-(prop-1-en-2-yl)icosahydro-3H-cyclopenta[5,6]naphtho[2,1-f]isoquinolin-3-yl)pyrimidine-5-carbonitrile hydrochloride (60 mg, 0.109 mmol), 4-(2-chloroethyl)thiomorpholine 1,1-dioxide hydrochloride (109 mg, 0.467 mmol), potassium phosphate (109 mg, 0.514 mmol) and sodium iodide (52.3 mg, 0.315 mmol) in acetonitrile (5 mL) was heated to 120° C. for 19 hours in a seal tube. The reaction mixture was diluted with methylene chloride (20 ml) and washed with water (20 ml). The organic layer was dried over sodium sulfate, filtered and concentrated under reduced pressure. The residue was purified by flash chromatography using a 0-40% acetone/hexanes gradient. The fractions containing the product were combine and concentrated under reduced pressure to give title compound (0.035 g, 0.052 mmol, 44%). LCMS: m/e 677.6 (M+H)⁺, 2.68 min (method 10B).

Step 4: To a suspension of 2-((4aR,6aR,6bR,8aS,11R,11aR,11bR,13aR,13bR)-8a-((2-(1,1-dioxidothiomorpholino)ethyl)amino)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carbonitrile (35 mg, 0.052 mmol) in ethanol (1 mL) was added a 40% w/w solution of KOH (2.0 mL, 12.0 mmol). The mixture was warmed to 80° C. for 3 hours then was cooled to rt and was purified by prep HPLC. The fractions containing the product were combined and concentrated to give 2-((4aR,6aR,6bR,8aS,11R,11aR,11bR,13aR,13bR)-8a-((2-(1,1-dioxidothiomorpholino)ethyl)amino)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyrimidine-5-carboxylic acid (0.020 g, 0.029 mmol, 56%) as a white solid. LCMS: m/e 696.7 (M+H)⁺, 2.15 min (method 3B). ¹H NMR (400 MHz, methanol-d₄) δ 8.79 (s, 2H), 4.83 (s, 1H), 4.73 (s, 1H), 4.42-4.33 (m, 1H), 3.67-3.55 (m, 1H), 3.27-2.76 (m, 12H), 2.22-2.00 (m, 4H), 1.76 (s, 3H), 1.65 (s, 3H), 1.55 (s, 3H), 1.20 (s, 3H), 1.14 (s, 3H), 1.94-1.08 (m, 19H), 1.03 (s, 3H)

### Example 54

### Preparation of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyridazine-3-carboxylic acid

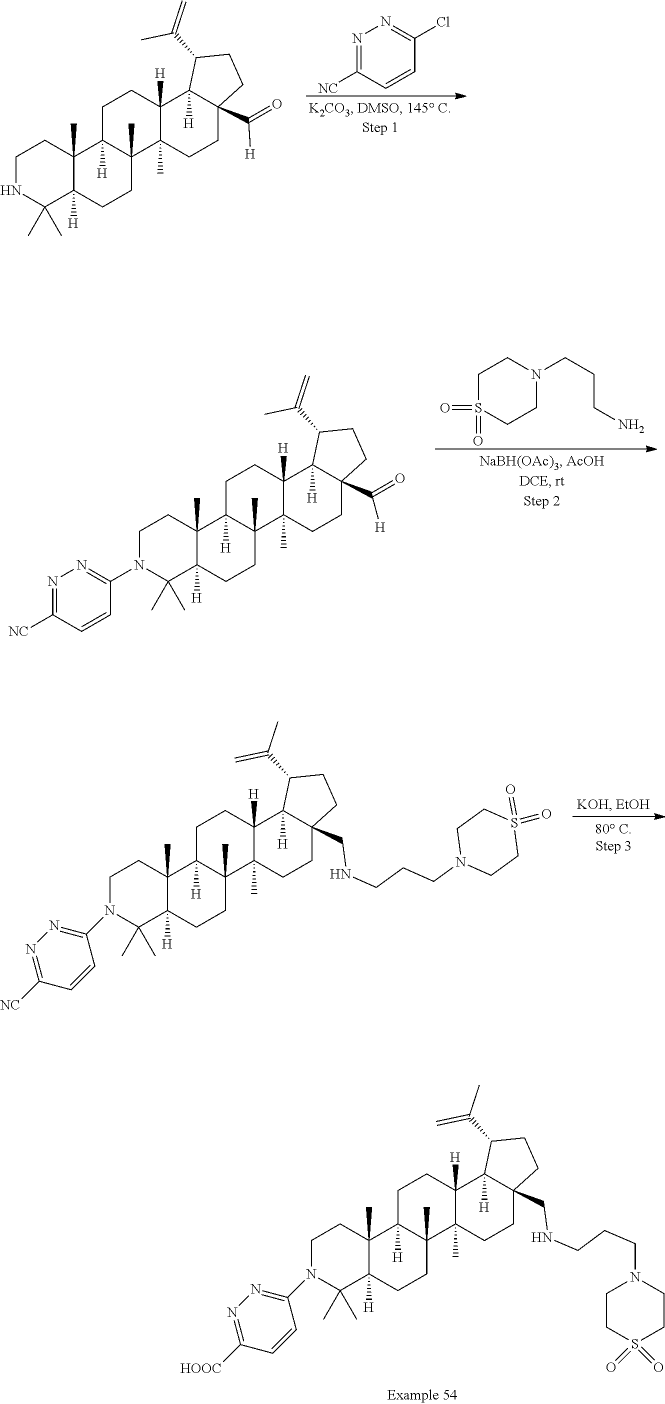

Step 1: Preparation of 6-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyridazine-3-carbonitrile. (4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde (50 mg, 0.117 mmol), 6-chloropyridazine-3-carbonitrile (164 mg, 1.175 mmol) and potassium carbonate (32.5 mg, 0.235 mmol) were added to a sealable pressure vessel and 4 drops of DMSO was added. The vessel was sealed and heated to 145° C. for 4 hours then was cooled to rt and diluted with dichloromethane and washed with water. The organic layer was dried over sodium sulfate, filtered and concentrated under reduced pressure. The residue was purified by flash chromatography. The fractions containing the product were combined and concentrated under reduced pressure to give the title product (0.025 g, 0.047 mmol, 40%) as a white solid. LCMS: m/e 529.5 (M+H)⁺, 2.51 min (method 3B)

Step 2: Preparation of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyridazine-3-carbonitrile.

To a solution of 6-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyridazine-3-carbonitrile (25 mg, 0.047 mmol) in DCE (2 mL) was added acetic acid (5.41 μl, 0.095 mmol) followed by 4-(3-aminopropyl)thiomorpholine 1,1-dioxide (18.18 mg, 0.095 mmol). The mixture was stirred at rt for 2 h, then sodium triacetoxyborohydride (50.1 mg, 0.236 mmol) was added and the mixture was stirred at rt overnight. The mixture was diluted with 7 mL of sat. sodium bicarbonate and was extracted with dichloromethane (3×7 mL). The combined organic layers were dried over sodium sulfate, filtered and concentrated under reduced pressure. The crude product (0.030 g, 0.043 mmol, 90%) was isolated as a yellow foam and was used in the next step without further purification. LCMS: m/e 705.7 (M+H)⁺, 2.28 min (method 10B).

Step 3: To a solution of 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyridazine-3-carbonitrile (24 mg, 0.034 mmol) in ethanol (1 mL) was added a 40% w/w solution of KOH (2.0 mL, 12.0 mmol) and the mixture was warmed to 80° C. for 3 hours. The mixture was cooled to rt and purified by prep HPLC. The fractions containing the expected product were combined and concentrated under reduced pressure to give 6-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)pyridazine-3-carboxylic acid t (0.023 g, 0.032 mmol, 94%) as a yellow solid. LCMS: m/e 724.7 (M+H)⁺, 2.07 min (method 3B). ¹H NMR (400 MHz, methanol-d₄) δ 8.13 (d, J=9.8 Hz, 1H), 7.78 (d, J=9.8 Hz, 1H), 4.76 (s, 1H), 4.65 (s, 1H), 3.73 (dd, J=8.0, 5.0 Hz, 2H), 3.67-3.59 (m, 4H), 3.47 (br. s., 4H), 3.29-3.13 (m, 5H), 2.86 (d, J=12.5 Hz, 1H), 2.58-2.44 (m, 1H), 2.27-1.98 (m, 3H), 1.73 (s, 3H), 1.58 (s, 3H), 1.58 (s, 3H), 1.14 (br. s., 3H), 1.08 (s, 3H), 1.01 (s, 3H), 1.96-0.96 (m, 21H).

### Example 55

### Preparation of 3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-hydroxycyclobut-3-ene-1,2-dione

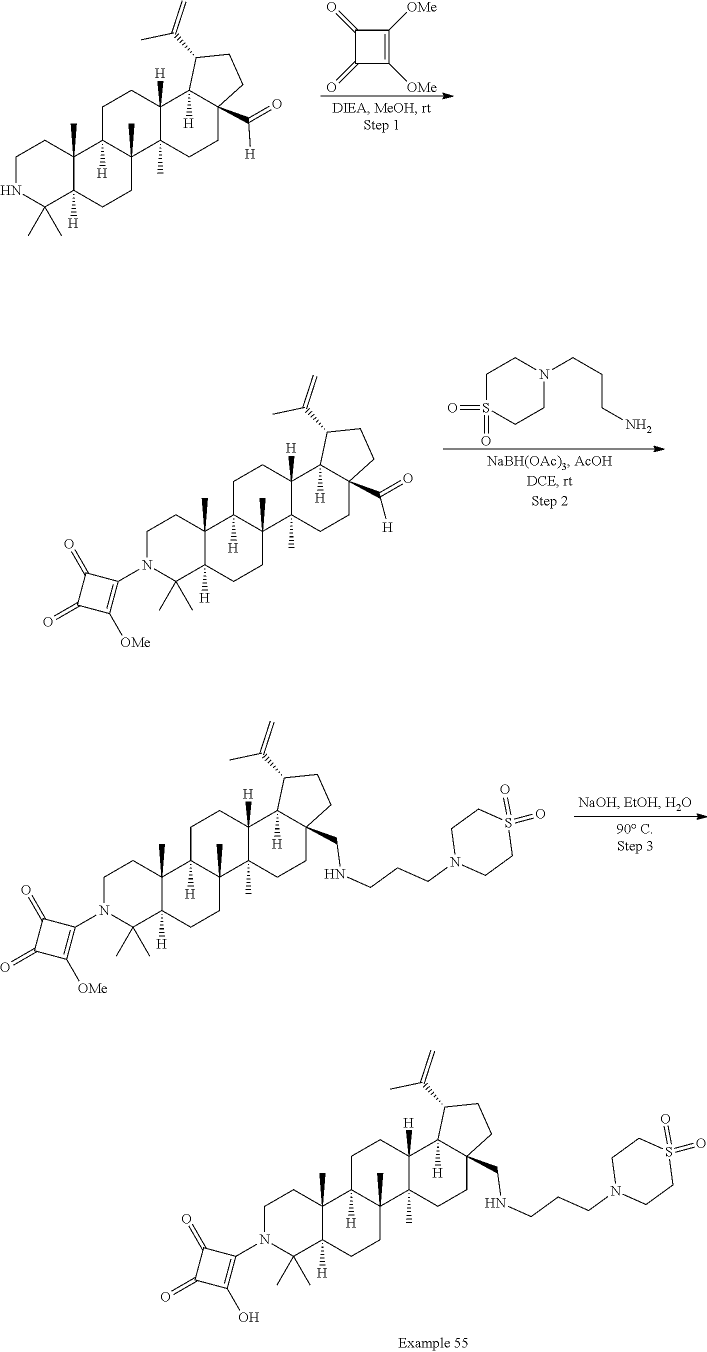

Step 1: Preparation of (4aR,6aR,6bR,8aS,11R,11aR,11bR,13aR,13bR)-3-(2-methoxy-3,4-dioxocyclobut-1-en-1-yl)-4,4,6a,6b,13b-pentamethyl-11-(prop-1-en-2-yl)icosahydro-1H-cyclopenta[5,6]naphtho[2,1-f]isoquinoline-8a-carbaldehyde.

To a 1-dram vial was added (4aR,6aR,6bR,8aS,11R,11aR,11bR,13aR,13bR)-4,4,6a,6b,13b-pentamethyl-11-(prop-1-en-2-yl)icosahydro-8aH-cyclopenta[5,6]naphtho[2,1-f]isoquinoline-8a-carbaldehyde (20.5 mg, 0.048 mmol) and 3,4-dimethoxycyclobut-3-ene-1,2-dione (26 mg, 0.183 mmol), followed by methanol (1.0 mL). To the mixture was added N,N-diisopropylethylamine (3 μl, 0.017 mmol). The solution was stirred at rt overnight then was concentrated under reduced pressure. The crude material was purified by flash chromatography using an ethyl acetate/hexanes gradient. The fractions containing the product were combined and concentrated to give the title compound as a white solid (0.022 g, 0.041 mmol, 85%). LCMS: m/e 536.5 (M+H)⁺, 5.00 min (method 11B).

Step 2: Preparation of 3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-methoxycyclobut-3-ene-1,2-dione.

To a solution of (4aR,6aR,6bR,8aS,11R,11aR,11bR,13aR,13bR)-3-(2-methoxy-3,4-dioxocyclobut-1-en-1-yl)-4,4,6a,6b,13b-pentamethyl-11-(prop-1-en-2-yl)icosahydro-1H-cyclopenta[5,6]naphtho[2,1-f]isoquinoline-8a-carbaldehyde (21 mg, 0.039 mmol) in DCE (2 mL) was added acetic acid (4.48 0.078 mmol) and 4-(3-aminopropyl)thiomorpholine 1,1-dioxide (15.07 mg, 0.078 mmol). The mixture was stirred at rt for 2 h, then sodium triacetoxyborohydride (41.5 mg, 0.196 mmol) was added and it was stirred at rt overnight. The mixture was diluted with 7 mL of sat. sodium bicarbonate and was extracted with dichloromethane (3×7 mL). The combined organic layers were dried over sodium sulfate, filtered and concentrated under reduced pressure. The crude product was used in the next step without further purification. LCMS: m/e 712.6 (M+H)⁺, 2.38 min (method 8B).

Step 3: 3-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)-4-methoxycyclobut-3-ene-1,2-dione dissolved in 0.5 ml of ethanol (0.5 mL) and sodium hydroxide (4.70 mg, 0.118 mmol) was added followed by water (0.500 mL). The suspension was stirred at 90° C. for two hours. The resulting mixture was purified by prep HPLC without work-up. Fractions containing desired product were combined and concentrated to afford the title product as a film (3 mg, 10.97%). LCMS: m/e 698.6 (M+H)⁺, 2.07 min (method 8B). ¹H NMR (400 MHz, methanol-d₄) δ 4.77 (br. s., 1H), 4.67 (s, 1H), 4.26-4.12 (m, 1H), 3.98-3.80 (m, 1H), 3.31-3.13 (m, 10H), 3.03-2.78 (m, 4H), 2.52-2.49 (m, 1H), 2.18-1.96 (m, 4H), 1.74 (s, 3H), 1.52 (s, 3H), 1.51 (s, 3H), 1.16 (s, 3H), 1.09 (s, 3H), 1.05 (s, 3H), 1.91-0.95 (m, 20H).

### Example 56

### Preparation of 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)thiazole-5-carboxylic acid

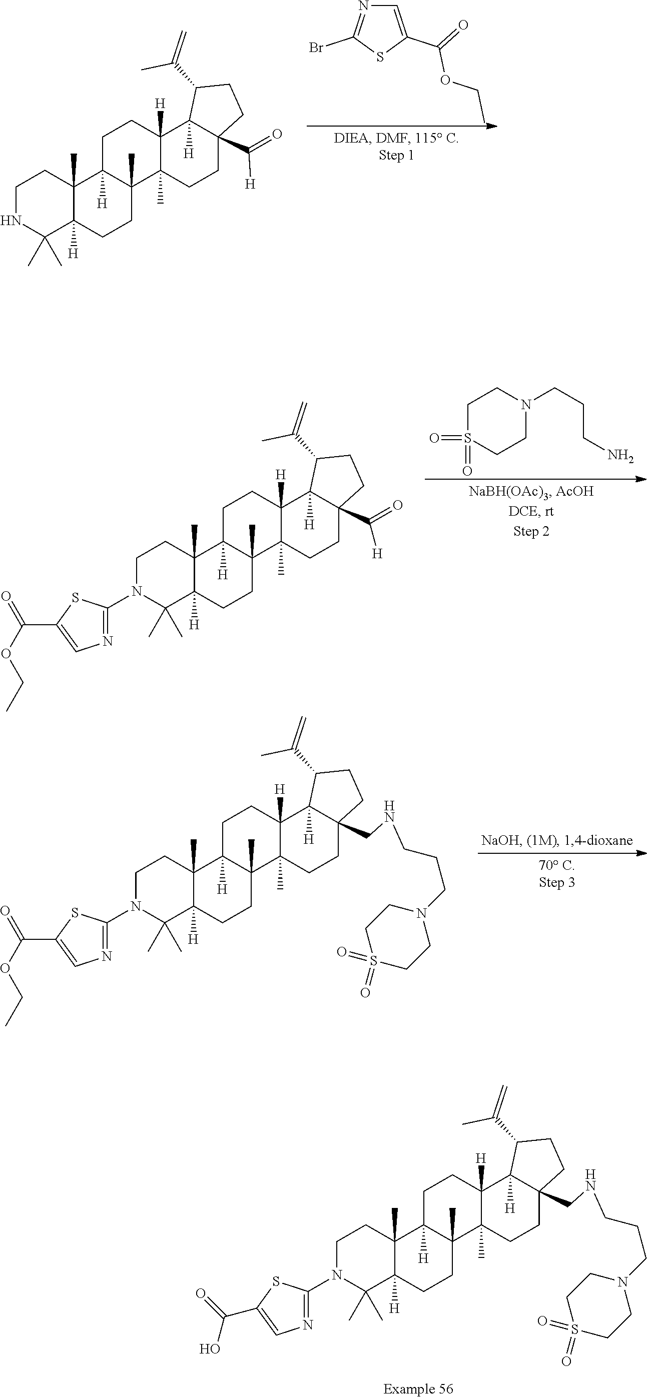

Step 1: Preparation of ethyl 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)thiazole-5-carboxylate. To a solution of (4aR,6aR,6bR,8aS,11R,11aR,11bR,13aR,13bR)-4,4,6a,6b,13b-pentamethyl-11-(prop-1-en-2-yl)icosahydro-1H-cyclopenta[5,6]naphtho[2,1-f]isoquinoline-8a-carbaldehyde (40 mg, 0.094 mmol) in DMF (2 mL) in a small resealable tube was added ⅓ the stated amount of ethyl 2-bromothiazole-5-carboxylate (222 mg, 0.940 mmol) and ½ the stated amount of N-ethyl-N-isopropylpropan-2-amine (450 4, 0.094 mmol). The solution was chilled to −78° C. followed by purging with nitrogen after evacuation to 30 micron Hg. The tube was sealed and warmed to 115° C. for 16 hours. An addition ⅓ of the bromide and ½ of the base were added and the tube sealed and returned to oil bath at 125° C. for 24 hours. The remaining ⅓ of the bromide was added and the reaction was allowed to proceed at 130° C. for 2 days. The solvent was removed under reduced pressure at 40° C. and the residue was purified by flash chromatography using a 0-20% ethyl acetate/hexanes gradient and a 25 g silica gel cartridge. The fractions containing the product were combined and concentrated under reduced pressure to give the title product as a pale, glassy solid (0.006 g, 0.010 mmol, 11%). LCMS: m/e 581.5 (M+H)⁺, 6.76 min (method 12B).

Step 2: Preparation of ethyl 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)thiazole-5-carboxylate.

To a solution of ethyl 2-((4aR,6aR,6bR,8aS,11R,11aR,11bR,13aR,13bR)-8a-formyl-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)thiazole-5-carboxylate (3.5 mg, 6.03 μmop in DCE (2 mL) was added acetic acid (2 equiv.) and 4-(3-aminopropyl)thiomorpholine 1,1-dioxide (2 equiv). The mixture was stirred at rt for 2 h, then to the mixture was added sodium triacetoxyborohydride (5 equiv.) and the mixture was stirred overnight at rt. The mixture was diluted with 7 mL of sat. sodium bicarbonate and was extracted with dichloromethane (3×7 mL). The combined organic layers were dried over sodium sulfate, filtered and concentrated under reduced pressure. The crude product was purified by pipet column, using a 0-8% ethyl acetate/hexanes gardient followed by a 0.5-6% methanol/dichloromethane gradient. Fractions containing the product were combined and concentrated under reduced pressure to give the title compound. LCMS: m/e 757.7 (M+H)⁺, 2.52 min (method 10B).

Step 3: To a solution of ethyl 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)thiazole-5-carboxylate (151 μg, 0.002 mmol) in dioxane (1 mL) was added sodium hydroxide (0.2 mL, 0.200 mmol). The mixture was warmed to 70° C. for 3 hours then the solvent was removed. The solid that remained was collected by filtration and washed with water to give 2-((4aR,6aR,6bR,8aS,11R,11aR,11bR,13aR,13bR)-8a-(((3-(1,1-dioxidothiomorpholino)propyl)amino)methyl)-4,4,6a,6b,13b-pentamethyl-11-(prop-1-en-2-yl)hexadecahydro-1H-cyclopenta[5,6]naphtho[2,1-f]isoquinolin-3(2H,4H,13bH)-yl)thiazole-5-carboxylic acid as a white solid (1.0 mg, 1.372 μmol, 69%). LCMS: m/e 729.6 (M+H)⁺, 2.09 min (method 10B).

Biology Data for the Examples

“μM” means micromolar;

“mL” means milliliter;

“μl” means microliter;

“mg” means milligram;

“μg” means microgram;

The materials and experimental procedures used to obtain the results reported in Table 1 are described below.

HIV cell culture assay—MT-2 cells and 293T cells were obtained from the NIH AIDS Research and Reference Reagent Program. MT-2 cells were propagated in RPMI 1640 media supplemented with 10% heat inactivated fetal bovine serum, 100 μg/ml penicillin G and up to 100 units/ml streptomycin. The 293T cells were propagated in DMEM media supplemented with 10% heat inactivated fetal bovine serum (FBS), 100 units/ml penicillin G and 100 μg/ml streptomycin. The proviral DNA clone of NL₄₋₃ was obtained from the NIH AIDS Research and Reference Reagent Program. A recombinant NL₄₋₃ virus, in which a section of the nef gene from NL₄₋₃ was replaced with the Renilla luciferase gene, was used as a reference virus. In addition, residue Gag P373 was converted to P373S. Briefly, the recombinant virus was prepared by transfection of the altered proviral clone of NL_{4-3.} Transfections were performed in 293T cells using LipofectAMINE PLUS from Invitrogen (Carlsbad, Calif.), according to manufacturer's instruction. The virus was titered in MT-2 cells using luciferase enzyme activity as a marker. Luciferase was quantitated using the Dual Luciferase kit from Promega (Madison, Wis.), with modifications to the manufacturer's protocol. The diluted Passive Lysis solution was pre-mixed with the re-suspended Luciferase Assay Reagent and the re-suspended Stop & Glo Substrate (2:1:1 ratio). Fifty (50) μL of the mixture was added to each aspirated well on assay plates and luciferase activity was measured immediately on a Wallac TriLux (Perkin-Elmer). Antiviral activities of inhibitors toward the recombinant virus were quantified by measuring luciferase activity in cells infected for 4-5 days with NLRluc recombinants in the presence serial dilutions of the inhibitor. The EC₅₀ data for the compounds is shown in Table 1.

TABLE 1

| Example | | EC₅₀ |
|---|---|---|
| # | Structure | (μM) |
| 1 | 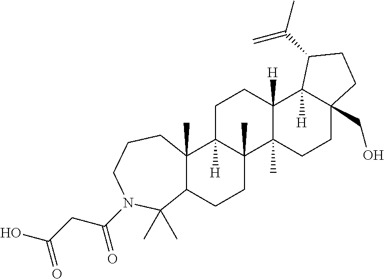 | 2.31 |
| 2 | 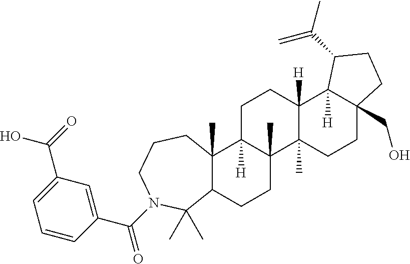 | >8.0 |
| 3 | 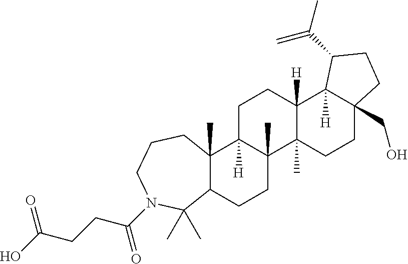 | >8.0 |
| 4 | 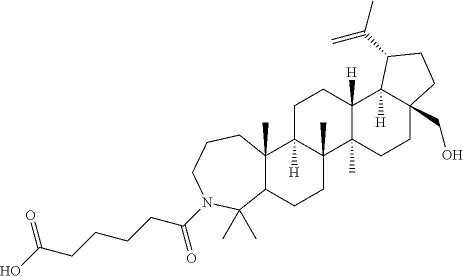 | >8.0 |
| 5 | 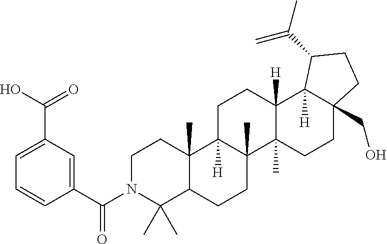 | 1.25 |
| 6 | 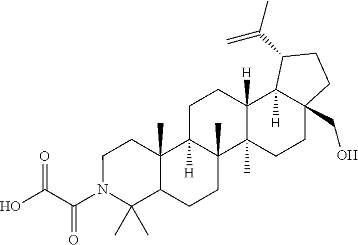 | 4.0 |
| 7 | 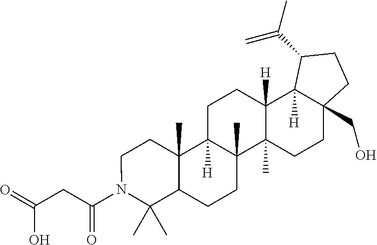 | 0.48 |
| 8 | 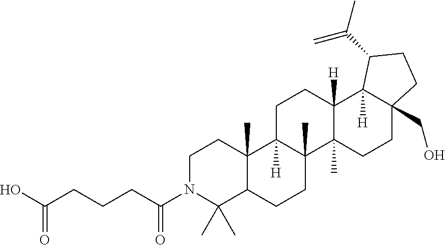 | 1.81 |
| 9 | 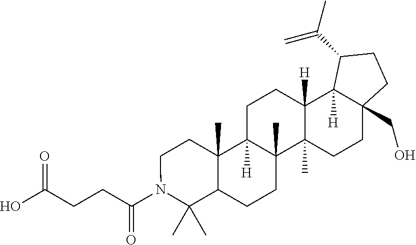 | 2.76 |
| 10 | 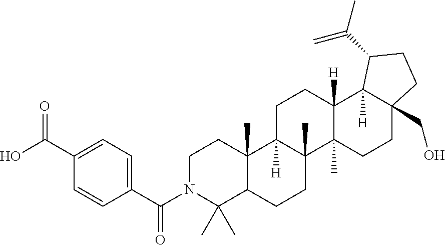 | 0.32 |
| 11 | 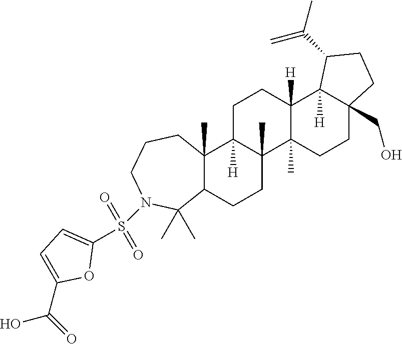 | >8.0 |
| 12 | 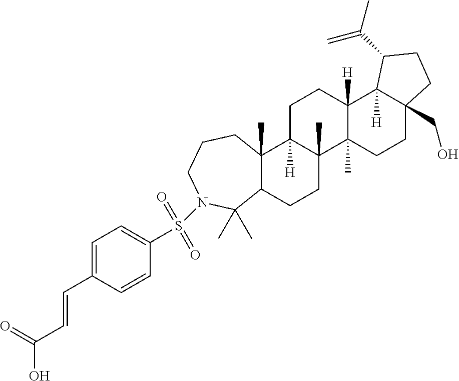 | >8.0 |
| 13 | 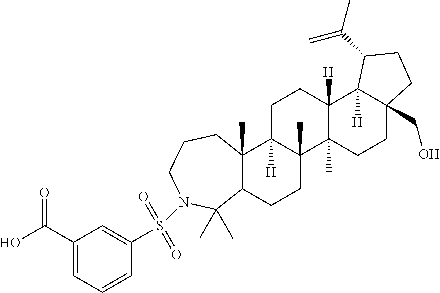 | >8.0 |
| 14 | 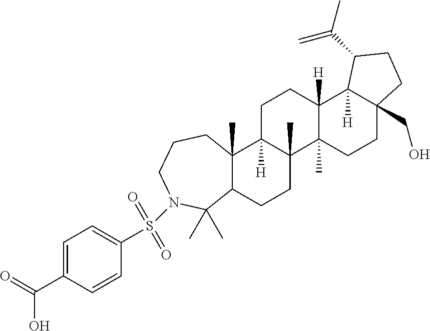 | 1.70 |
| 15 | 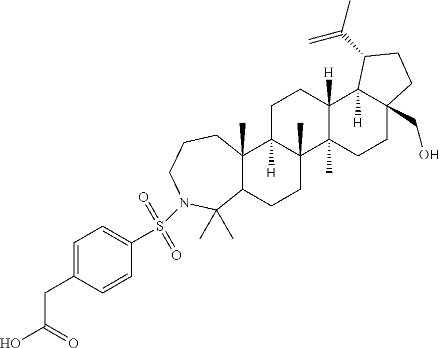 | >8.0 |
| 16 | 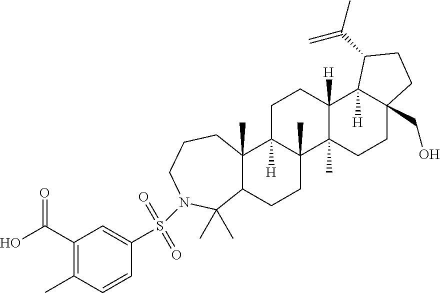 | >8.0 |
| 17 | 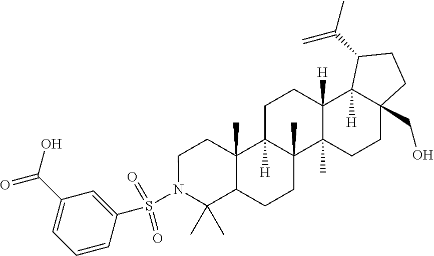 | 4.94 |
| 18 | 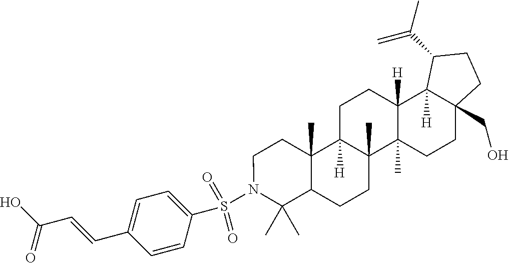 | 4.2 |
| 19 | 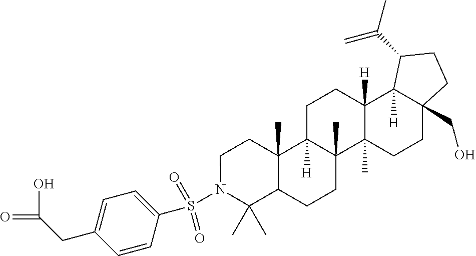 | >8.0 |
| 20 | 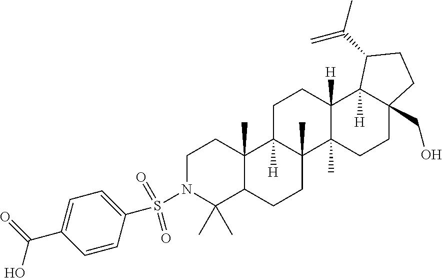 | 3.1 |
| 21 | 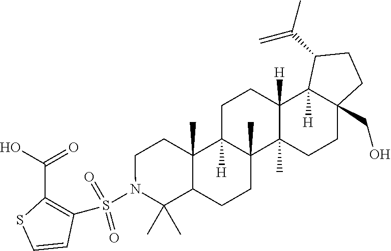 | >8.0 |
| 22 | 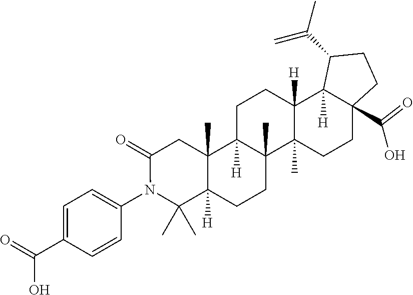 | >3.0 |
| 23 | 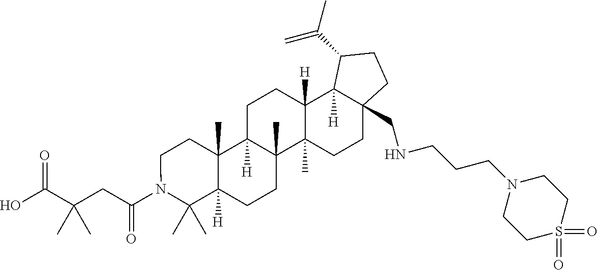 | 0.39 |
| 24 | 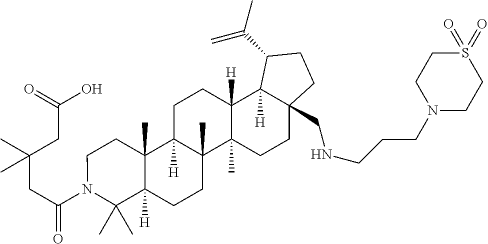 | 0.12 |
| 25 | 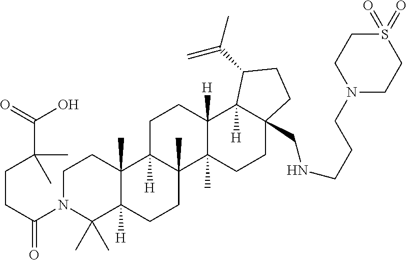 | 0.12 |
| 26 | 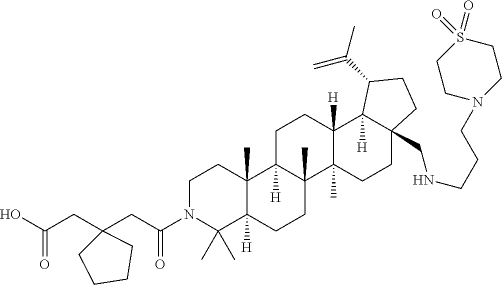 | 0.13 |
| 27 | 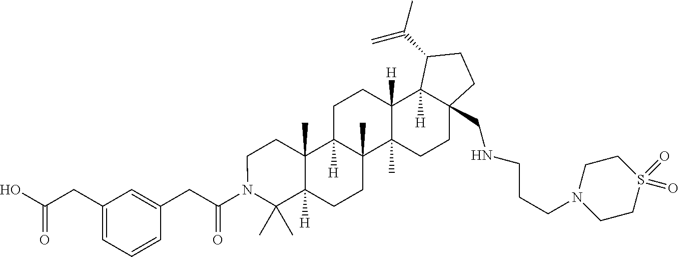 | 0.3 |
| 28 | 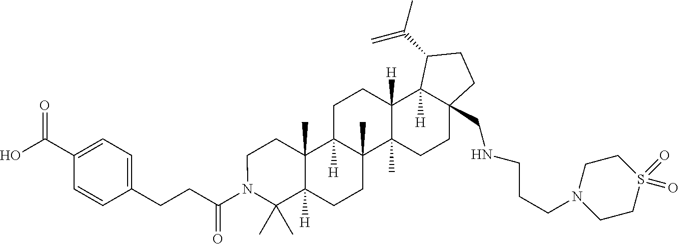 | 0.94 |
| 29 | 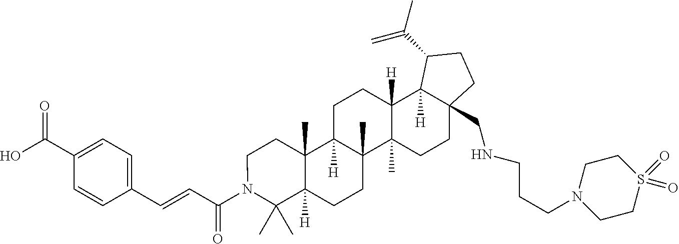 | 1.59 |
| 30 | 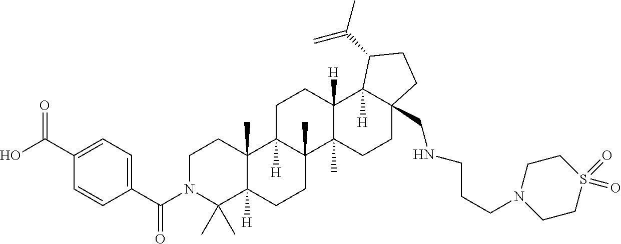 | 0.11 |
| 31 | 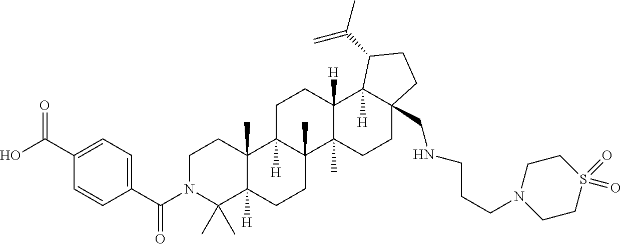 | >3.0 |
| 32 | 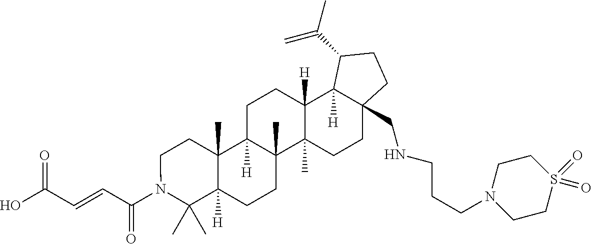 | 0.30 |
| 33 | 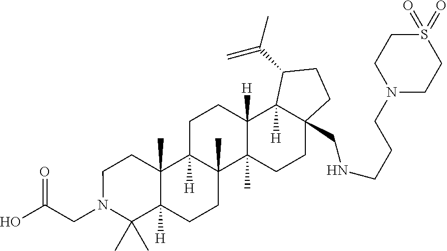 | >3.0 |
| 34 | 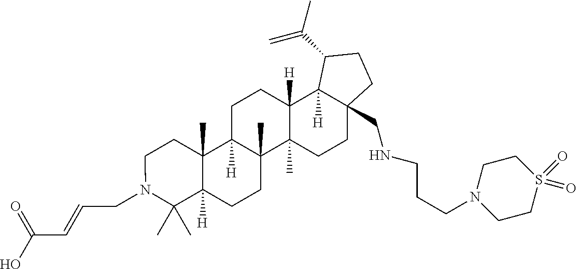 | >3.0 |
| 35 | 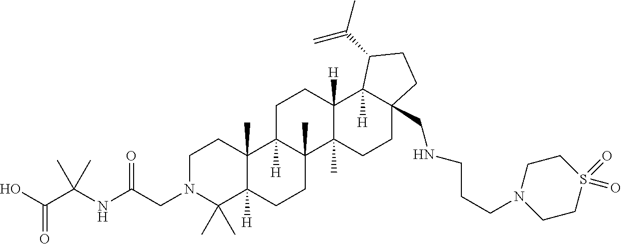 | >3.0 |
| 36 | 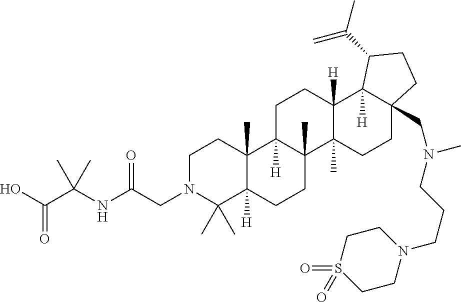 | 1.3 |
| 37 | 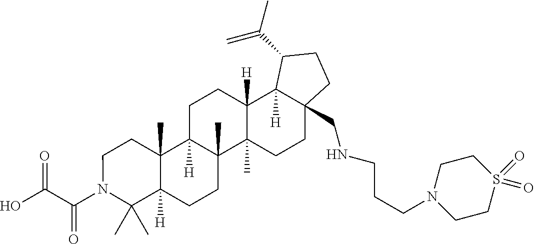 | >3.0 |
| 38 | 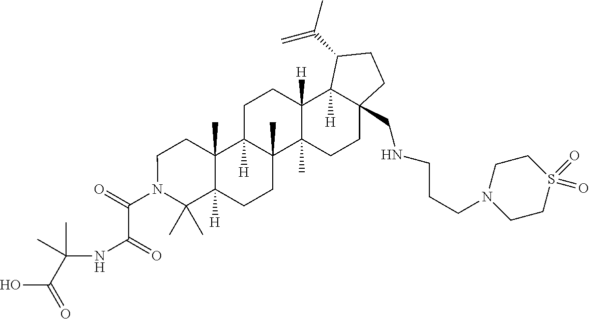 | >3.0 |
| 39 | 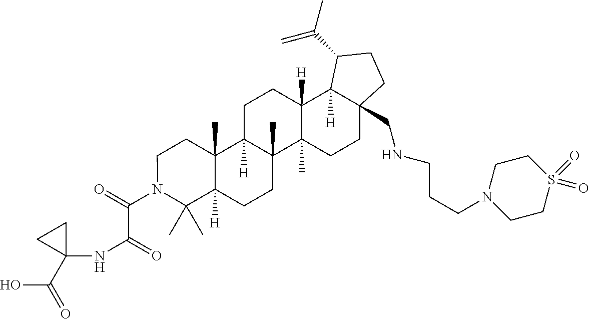 | >3.0 |
| 40 | 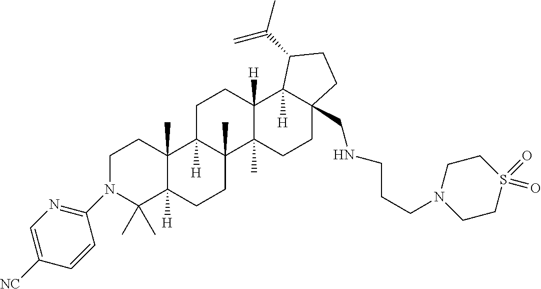 | 0.76 |
| 41 | 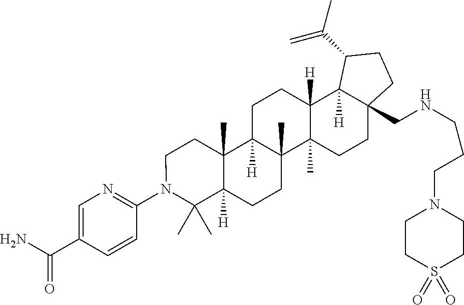 | 0.4 |
| 42 | 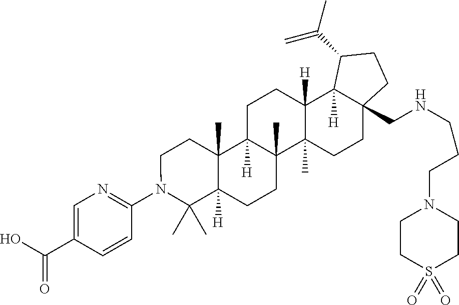 | 0.003 |
| 43 | 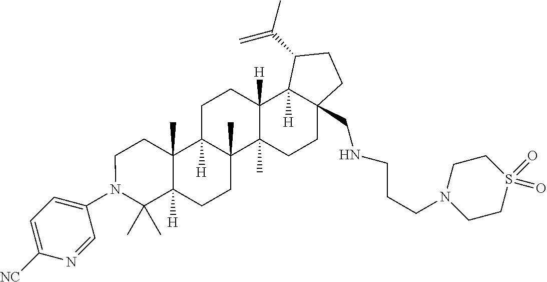 | 0.28 |
| 44 | 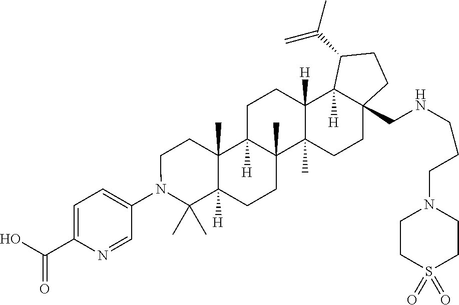 | 0.007 |
| 45 | 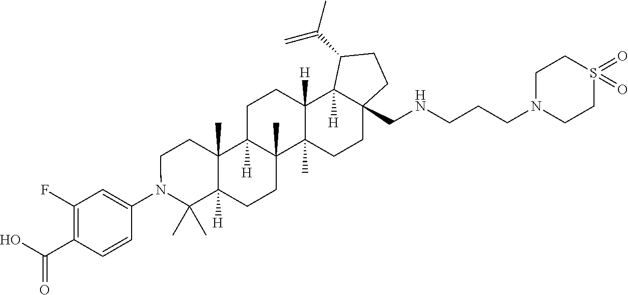 | 0.02 |
| 46 | 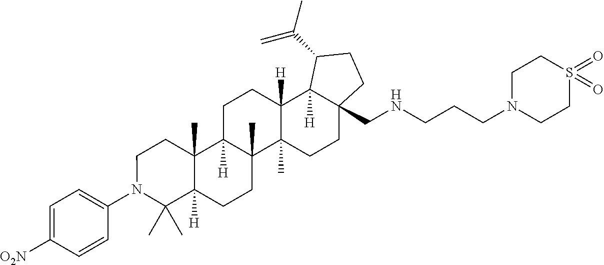 | >0.33 |
| 47 | 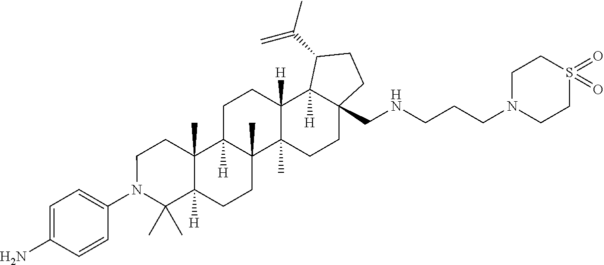 | >0.33 |
| 48 | 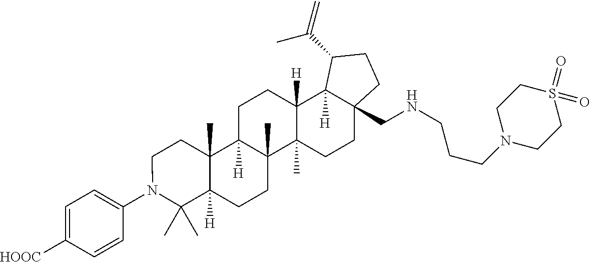 | 0.005 |
| 49 | 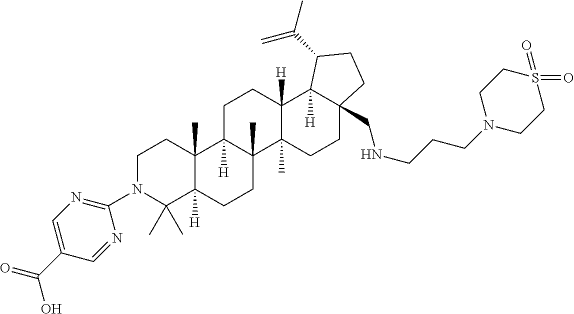 | 0.0007 |
| 50 | 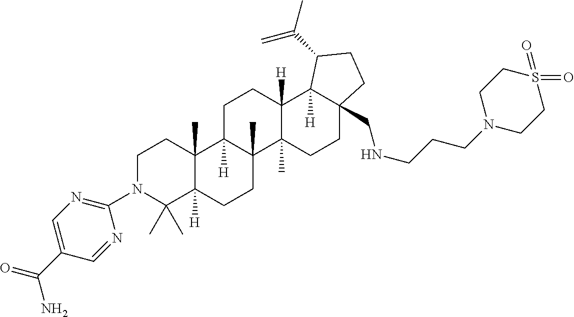 | 0.06 |
| 51 | 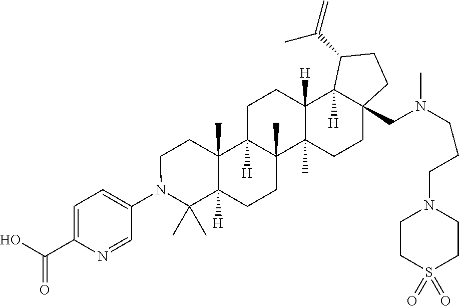 | 0.026 |
| 52 | 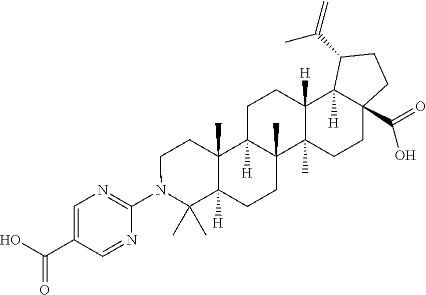 | 0.062 |
| 53 | 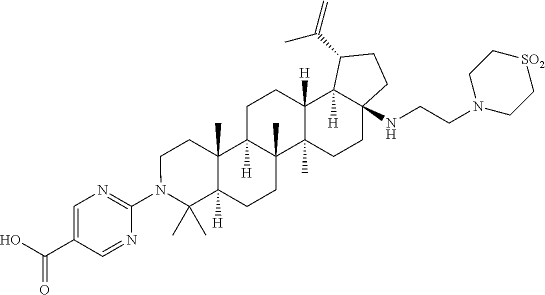 | 0.001 |
| 54 | 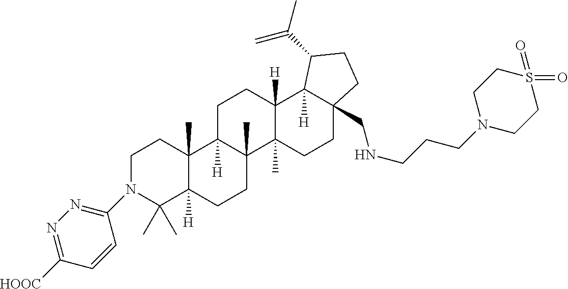 | 0.28 |
| 55 | 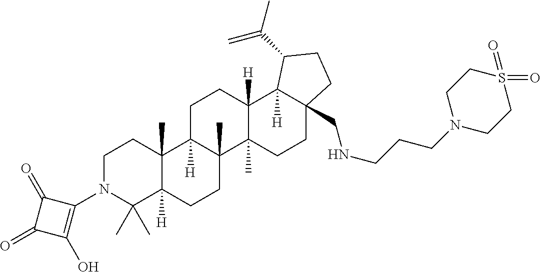 | 0.25 |
| 56 | 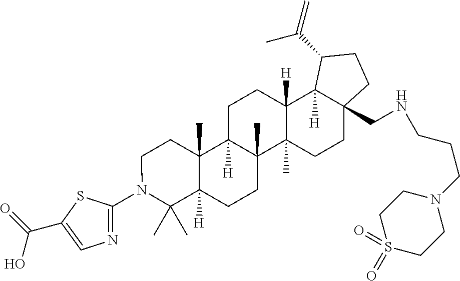 | 0.004 |

The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

1. (canceled)

2. A compound or salt as claimed in claim 28, wherein R₁ is isopropenyl.

3-9. (canceled)

10. A compound or salt as claimed in claim 28, wherein Y is —COOH.

11-17. (canceled)

18. A compound or salt as claimed in claim 28, wherein W is —CH₂OR₂.

19.-23. (canceled)

24. A compound or salt as claimed in claim 28 wherein W is —CH₂NR₂₆R₂₇.

25-27. (canceled)

28. A compound of Formula II, or a pharmaceutically acceptable salt thereof: 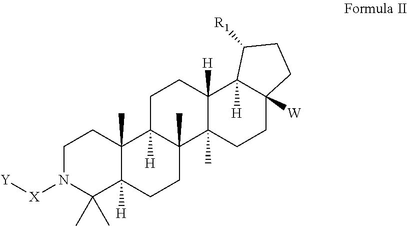
wherein R₁ is isopropenyl or isopropyl;
X is a phenyl or heteroaryl ring substituted with A, wherein A is at least one member selected from —H, halogen, hydroxyl, —C₁₋₆ alkyl, —C₂₋₆ alkenyl, —C₁₋₆ alkoxy, and —COOR₂;
alternatively, X is selected from a single bond, —C₁₋₆alkyl-, —C₁₋₆alkylaryl-, —C₂₋₆alkenyl-, —C₂₋₆alkenylaryl-, —CO—, —SO₂—, —C₁₋₆alkylCO—, —C₂₋₆alkenylCO—, —COalkylsubstituted C₁₋₆ alkyl-, —COspiroalkylsubstitutedC₁₋₆ alkyl-, —COarylsubstitutedC₁₋₆ alkyl-, —COarylsubstituted C₂₋₆ alkenyl-, —COheteroaryl-, —COaryl-, —COC₁₋₆ alkylaryl-, —COC₁₋₆ alkylheteroaryl-, —COalkylsubstituted C₃₋₆ cycloalkyl-, —COC₂₋₆ alkenylaryl-, —COC₁₋₆ alkyl(NHR₀)—, —C₁₋₆ alkyl(CONHR₀)—, —(COCO)NR₀SO₂—, —SO₂C₁₋₆ alkyl-, —SO₂alkylsubstitutedC₁₋₆ alkyl-, —SO₂arylsubstituted C₁₋₆ alkyl-, —SO₂C₁₋₆ alkylaryl-, —SO₂C₂₋₆ alkenylaryl-, —SO₂aryl-, —SO₂arylC₁₋₆alkyl-, —SO₂arylC₂₋₆alkenyl-, —SO₂heteroaryl-, -heteroaryl-Q₀- and -aryl-Q₀-;
Q₀ is selected from —C₁₋₆ alkyl, -halo, —CF₃ and —OC₁₋₆ alkyl,
R₀ is H, —C₁₋₆ alkyl, -alkylsubstituted C₁₋₆ alkyl, -alkylsubstituted(C₁₋₆)COOR₆, -spiroalkylsubstituted(C₁₋₆)COOR₆, or -aryl substituted C₁₋₆ alkyl;
R₂ is —H, —C₁₋₆ alkyl, -alkylsubstituted C₁₋₆ alkyl, or-arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₚ—COOR₂, —SO₂NR₂C(O)R₂, -tetrazole, and —CONHOH,
wherein p is 1-6;
alternatively, Y is selected from a phenyl or heteroaryl ring, optionally further substituted with 1 to 3 substituents selected from —H, -halo, -hydroxyl, —C₁₋₆ alkyl, —C₁₋₆ alkoxy, —COOR₂, —CN, —NO₂, —CF₃, —SO₂, —NR₂₆R₂₇, —CONR₂₆R₂₇, and —SO₂NR₂₆R₂₇;
alternatively, —X-Y is selected from 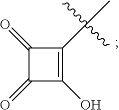 [SMILES: *c1c(O)c(=O)c1=O]
R₃ is —C₁₋₆ alkyl or -alkylsubstituted C₁₋₆ alkyl;
Z is selected from —CO— and —CH₂—;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, and —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ heterocycle, aryl, heteroaryl, substituted heteroaryl, halogen, —CF₃, —OR₂, —COOR₂, —NR₈R₉, —CONR₁₀R₁₁ and —SO₂R₇;
alternatively, R₄ is selected from —C₃₋₆ cycloalkyl, —C₁₋₆ substituted alkyl, —C₁₋₆ alkyl-heteroaryl, —C₁₋₆ alkyl-substituted heteroaryl, —C₁₋₆ alkyl-NR₆R₇, —C₁₋₆ alkyl-CONR₈R₉, —C₃₋₆ cycloalkyl-CONR₈R₉, —C₃₋₆ cycloalkyl-(CH₂)₁₋₃—NR₆R₇, —(CH₂)₁₋₃—C₃₋₆ cycloalkyl-NR₆R₇, —(CH₂)₁₋₃—C₃₋₆ cycloalkyl-(CH₂)₁₋₃—NR₆R₇; —C₁₋₆ alkyl-Q′₁, C₃₋₆ cycloalkyl-Q₁, —COR₁₀, —SO₂R₃, and 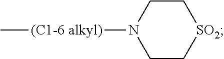
wherein Q′₁ is selected from-hydroxy, —COOR₂, -halo, and —SO₂Rₐ;
Rₐ is C₁₋₆ alkyl, NR₂R₂, 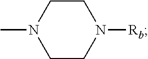
R_{b} is —H, —C₁₋₆ alkyl, —COR₃, —SO₂R₃, —SONR₃R₃,
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substituted cycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ heterocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
alternatively R₈ and R₉ are taken together with the adjacent N to form a cycle selected from: 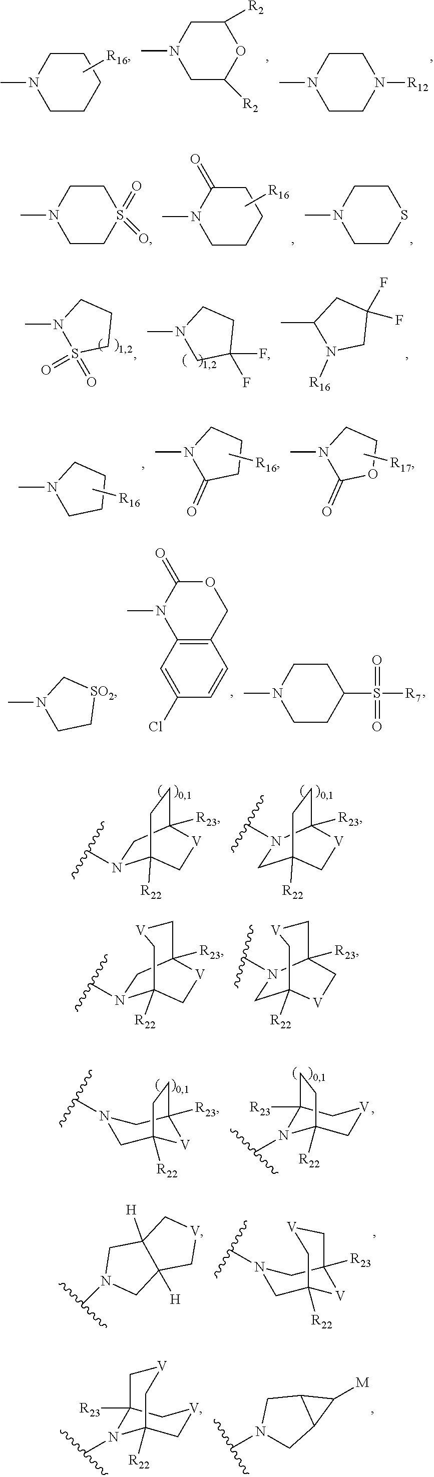 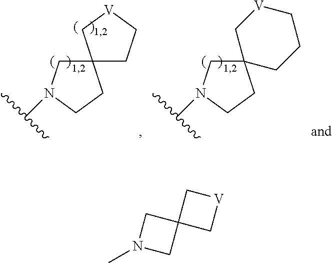
V is selected from —CR₂₄R₂₅—, —SO₂—, —O— and —NR₁₂—;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
alternatively R₁₀ and R₁₁ are taken together with the adjacent N to form a cycle such as 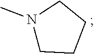
R₁₂ is selected from —C₁₋₆ alkyl, —C₁₋₆ alkyl-OH; —C₁₋₆ alkyl, —C₁₋₆ substituted alkyl,-C₃₋₆ cycloalkyl, —COR₇, —COONR₂₂R₂₃, —SORT, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
alternatively R₁₃ and R₁₄ are taken together with the adjacent N to form a cycle selected from: 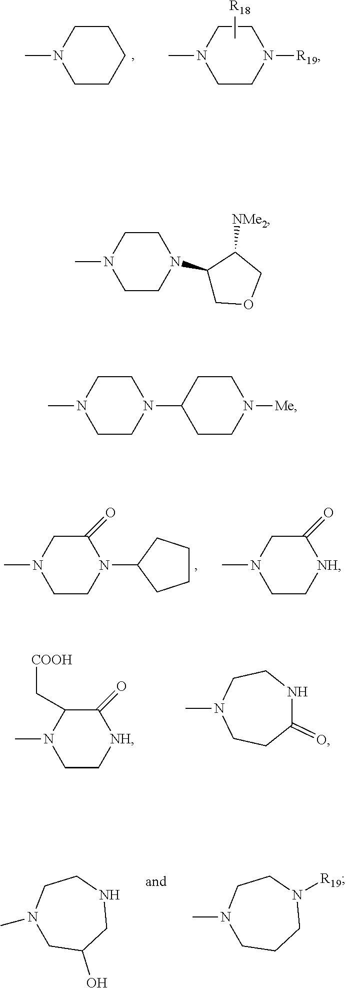
Q₃ is selected from heteroaryl, substituted heteroaryl, —NR₂₀R₂₁, —CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃,
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃,
wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
alternatively R₂₀ and R₂₁ are taken together with the adjacent N to form a cycle selected from 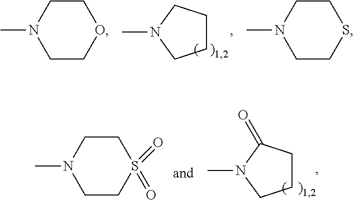
with the proviso that only one of R₂₀ or R₂₁ can be —COR₃,
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from 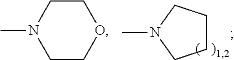
R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl,
and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂, and 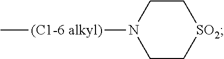
or alternatively R₂₆ and R₂₇ are taken together with the adjacent N to form a cycle selected from:

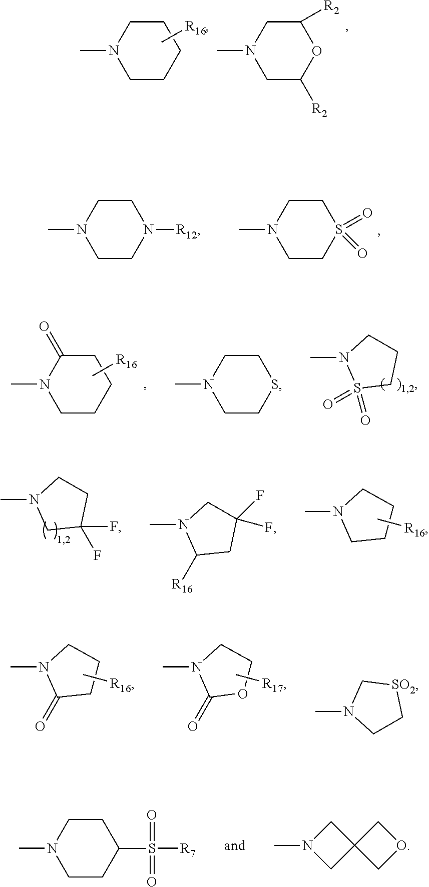

29. (canceled)

30. A pharmaceutical composition comprising a compound or salt of claim 28 and a pharmaceutically acceptable carrier.

31. The composition of claim 30 further comprising a at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

32. The composition of claim 30 wherein the other agent is dolutegravir.

33. A method for treating HIV infection comprising administering a compound of claim 28, or a pharmaceutically acceptable salt thereof, to a patient in need thereof.

34. The method of claim 33 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

35. The method of claim 34 wherein the other agent is dolutegravir.

36. The method of claim 34 wherein the other agent is administered to the patient prior to, simultaneously with, or subsequently to the compound or salt of claim 28.
